(19) United States
(12) Patent Application Publication
(10) Pub. No.: US 2023/0193181 A1
(43) Pub. Date: Jun. 22, 2023

(54) 3D Tissue Printing

(71) Applicant: Oxford University Innovation Limited, Oxford (GB)

(72) Inventors: John Hagan Pryce Bayley, Stonesfield Oxfordshire (GB); Linna Zhou, Kidlington, Oxfordshire (GB)

(21) Appl. No.: 17/999,286

(22) PCT Filed: May 20, 2021
(86) PCT No.: PCT/GB2021/051225

§ 371 Date: Nov. 18, 2022

(30) Foreign Application Priority Data
May 20, 2020 (GB) 2007478.7

(51) Int. Cl. C12M 1/12; C12M 1/26; C12N 11/04

(52) U.S. Cl. CPC C12M 25/01; C12M 25/16; C12M 33/00; C12N 11/04

(57) ABSTRACT

The invention relates to an apparatus for producing a droplet assembly, which apparatus comprises: at least one droplet generator suitable for generating droplets of a viscous droplet medium; a droplet receiving region which is moveable relative to the at least one droplet generator; a temperature controller; and a control unit, which control unit is adapted to control the dispensing of droplets from the at least one droplet generator and the movement of the droplet receiving region relative to the at least one droplet generator, wherein the apparatus is adapted to produce a droplet assembly in the droplet receiving region, wherein the droplet assembly comprises a plurality of droplets, wherein each of said droplets comprises (i) a droplet medium, and (ii) an outer layer of amphipathic molecules around the surface of the droplet medium.

### FIELD OF THE INVENTION

The invention relates to apparatus for producing a droplet assembly, processes for producing a droplet assembly, a process for producing a pre-patterned tissue construct, and a process for producing a cultured tissue construct. The invention also relates to a droplet assembly, a pre-patterned tissue construct and a cultured tissue construct. The invention also relates to a droplet array and a process for producing a droplet array. This invention also relates to a nano-bioreactor, an array of nano-bioreactors and a process for producing a nano-bioreactor.

### INTRODUCTION

Human tissues consist of complex cellular patterns and structures that arise during development and are essential for tissue function.[1] Some of these self-organised patterns have been recreated in vitro, in 2D and 3D cell culture.[2, 3] For example, human organoids derived from embryonic stem cells or induced pluripotent stem cells (iPSCs) are valuable tools for developmental biology, disease modelling and regenerative medicine.[3, 4] However, these structures generated from homogenous and uncontrolled cell aggregates spontaneously organise overtime in an unpredictable and spatially disordered manner. Thus far, robust technologies have not existed that could control initial 3D cell patterns to study how this influences development and functions. The ability to pre-position cell types in the initial state of the self-organisation process could allow greater control over subsequent organised states. Geometric confinement with patterned surfaces for cell attachment at the onset of tissue self-organisation can affect later-stage stem/progenitor cell self-organisation events such as embryonic germ layer patterning [5] and neural rosette formation [6]. However, these approaches were conducted on 2D micro-fabricated surfaces and have not yet been extended to produce defined 3D geometries. Manually generated pre-patterned 3D structures, for example assembled brain organoids[7] or functionally distinct cell clusters,[8] can be powerful tools for the study of neuronal migration across brain regions and brain region specification induced by signalling molecules. The mechanical automation of these spatial programming processes would increase the reproducibility and complexity of induced self-organisation in 3D tissue models.

Human brain development is poorly understood due to lack of models and experimental tools. It is also one of the softest tissues and has remained a challenge for bio-printing. 3D bio-printing offers a fast pre-patterning method but so far applications have been limited to the construction of tissues that are stiffer than the brain.[9, 10] Efforts towards printing soft tissues have involved the addition of hard scaffold materials for mechanical support or have been limited to flat structures.[9, 11, 12] Neural stem cell (NSC) printing has relied on the incorporation of polysaccharides, such as agarose/alginate/gellan gum[13-16] or synthetic polymer-based scaffolds[17, 18]. 3D printing of tens of thousands of picoliter aqueous droplets conjoined by lipid bilayers to give 3D tissue-like materials has previously been reported.[19] Further, printed cellular structures were achieved by using agarose as matrix to give cartilage-like constructs.[20] The majority of the matrices and scaffolds used for bio-printing have not recreated the functional properties and complexity of extracellular matrix (ECM). Therefore, other methods are yet to be tested for interrogating how initial cell positions trigger self-organisation or recapitulate human brain developmental events. There therefore exists a need to develop 3D printing techniques which are suitable for printing materials that realistically mimic soft tissues, without the need for extraneous scaffolds and which provide good control over the positioning and pattern of cells in the printed material.

### SUMMARY OF THE INVENTION

To 3D print naturalistic and also artificially pre-patterned brain tissues, an enhanced droplet printing apparatus and process was developed that enables the construction of soft tissues with ECM and without hard materials to affect subsequent self-organisation. Printed NSCs in ECM were viable, differentiated and were functional. By spatially arranging NSCs inside Matrigel (basement membrane derived ECM), a series of cortical developmental events could be triggered: neuronal migration, differentiation, axon outgrowth and astrogenesis. Further, pre-patterned astrocytes (surrounding NSCs) induced robust axonal fasciculation, suggesting that astrocytes participate in neural tract formation. Differentiated cortical tissues were rapidly produced. Finally, spatial pre-patterning can be used to investigate cell migration in cortical tissues, which revealed that astrocytes preferentially maintained segregation from neurons indicating non-reciprocal chemorepulsion between neurons and astrocytes. Therefore, the printing technique described herein provides 3D pre-determined cell patterning, enabling insights into subsequent self-organisation processes that are important in cortical development such as axonal fasciculation and cell migration/segregation. In particular, the printing apparatus and process of the invention permit printing with materials that better mimic in vivo conditions, for instance natural extracellular matrix materials, without the need for additional supporting materials in the structure.

The printing technique also permits the production of arrays of small elements containing small numbers of cells in ECM material that find utility in high-throughput screening. Microfluidics are able to produce large quantities of cell-containing droplets with precise control in droplet size and content for biomedical applications. However, limited droplet patterns have been demonstrated with microfluidics to study cell functions in tissue-mimicking microenvironments with both cell-extracellular matrix (ECM) and cell-cell interactions. The invention provides a droplet-printing process to generate low picoliter to nanoliter droplet arrays with cells and natural materials such as ECM, Matrigel or collagen. Various 3D patterned droplet assemblies, containing multiple patterned droplets, were produced in arrays. We demonstrated that functional human primary cortical astrocytes were printed with ECM. Printed astrocytes showed higher frequent spontaneous calcium fluctuations than in 2D culture, while the interaction with metastatic cancer cells, MDA, decreased these spontaneous calcium fluctuations. Patterned droplet assemblies containing either astrocytes or MDA cells, showed different amount of increase in the intracellular calcium levels of astrocytes, indicating a cell-cell interaction dependent functional response of astrocytes towards cancer cells. Therefore, the droplet printing process of the invention can be applied in the production of arrays of defined micrometre-scale cellular patterns for studying cell-ECM and cell-cell interactions. The printed arrays of the invention may also find use in screening experiments, by permitting small amounts of different test substances to be screened with live cells in vitro.

Accordingly, the invention provides an apparatus for producing a droplet assembly, which apparatus comprises: at least one droplet generator suitable for generating droplets of a viscous droplet medium; a droplet receiving region which is moveable relative to the at least one droplet generator; a temperature controller; and a control unit, which control unit is adapted to control the dispensing of droplets from the at least one droplet generator and the movement of the droplet receiving region relative to the at least one droplet generator, wherein the apparatus is adapted to produce a droplet assembly in the droplet receiving region, wherein the droplet assembly comprises a plurality of droplets, wherein each of said droplets comprises a droplet medium.

The invention also provides a process for producing a droplet assembly using an apparatus for producing the droplet assembly, which droplet assembly comprises: a plurality of droplets, wherein each of said droplets comprises a droplet medium, which apparatus comprises: at least one droplet generator wherein the droplet generator is suitable for generating droplets of a viscous droplet medium; a droplet receiving region which is moveable relative to the at least one droplet generator; a temperature controller; and a control unit, which control unit is adapted to control the dispensing of droplets from the at least one droplet generator and the movement of the droplet receiving region relative to the at least one droplet generator; wherein said droplet receiving region further comprises a bulk medium, wherein the bulk medium and the droplet medium are immiscible; which process comprises:

(a) a plurality of dispensing steps, wherein each dispensing step comprises dispensing a droplet of the droplet medium from a said droplet generator into the bulk medium, and thereby forming in the bulk medium a droplet which comprises said droplet medium; and

(b) moving the droplet receiving region relative to the at least one droplet generator, to control the relative positioning of the droplets in the bulk medium.

The invention also provides a droplet assembly which is obtainable by a process of the invention as defined above and optionally as further defined herein.

The invention also provides a process for producing a droplet assembly, the process comprising generating, in a bulk medium, a plurality of droplets, wherein each of said droplets comprises: (i) a droplet medium which comprises biological cells and a natural extracellular matrix material, and (ii) an outer layer of amphipathic molecules around the surface of the droplet medium, wherein the bulk medium and the droplet medium are immiscible, and contacting each of said droplets with another of said droplets to form a layer of said amphipathic molecules as an interface between contacting droplets.

The invention also provides a droplet assembly which is obtainable by a process of the invention as defined above and optionally as further defined herein.

The invention also provides a process for producing a pre-patterned tissue construct, the process comprising
producing a pre-patterned droplet assembly in a bulk medium by a process as defined herein, or providing a pre-patterned droplet assembly as defined herein, provided that, in the pre-patterned droplet assembly, the droplet medium comprises natural extracellular matrix material and biological cells;
gelling the natural extracellular matrix material to produce a pre-patterned tissue construct which comprises gelled natural extracellular matrix material and the biological cells; and
recovering the pre-patterned tissue construct from the bulk medium.

The invention also provides a pre-patterned tissue construct which is obtainable by a process of the invention as defined above and optionally as further defined herein.

The invention also provides a process for producing a cultured tissue construct comprising
preparing a pre-patterned tissue construct by the process of the invention for producing a pre-patterned tissue construct; and
culturing the biological cells in the pre-patterned tissue construct.

The invention also provides a cultured tissue construct which is obtainable by the process of the invention for producing a cultured tissue construct.

The invention also provides a droplet assembly comprising: a plurality of droplets in contact with one another, wherein each of said droplets comprises: (i) a droplet medium which comprises biological cells and a natural extracellular matrix material, and (ii) an outer layer of amphipathic molecules around the surface of the droplet medium, wherein each of said droplets contacts another of said droplets to form a layer of said amphipathic molecules as an interface between the contacting droplets.

The invention also provides a pre-patterned tissue construct comprising a plurality of gelled droplets in contact with one another, wherein each of said droplets comprises a droplet medium which comprises biological cells and a gelled natural extracellular matrix material, and wherein each of said droplets is adhered to another of said droplets by the gelled natural extracellular matrix material.

The invention also provides a cultured tissue construct which comprises a natural extracellular matrix material and biological cells, wherein the cultured tissue construct is obtainable by providing a pre-patterned tissue construct of the invention and culturing the biological cells in the pre-patterned tissue construct.

The invention also provides a droplet array which comprises a plurality of elements spaced apart from one another on a substrate in a bulk medium, wherein each element comprises at least one droplet which comprises a droplet medium which comprises one or more biological cells and a natural extracellular matrix material, wherein the bulk medium and the droplet medium are immiscible, optionally wherein each element comprises at least one droplet which comprises (i) a droplet medium which comprises one or more biological cells and a natural extracellular matrix material and (ii) an outer layer of amphipathic molecules.

The invention also provides a droplet array which comprises a plurality of elements spaced apart from one another on a substrate, wherein each element comprises at least one gelled droplet, wherein each gelled droplet comprises a droplet medium which comprises one or more biological cells and a gelled natural extracellular matrix material.

The invention also provides the use of a droplet array as described herein in high throughput screening.

The invention also provides a method of screening a test substance which comprises providing a droplet array of the invention, contacting the test substance with at least one of the elements of the droplet array, and measuring a response.

The invention also provides a process for producing a droplet array, which droplet array comprises a plurality of elements spaced apart from one another on a substrate in a bulk medium, wherein each element comprises at least one droplet which comprises a droplet medium which comprises one or more biological cells and a natural extracellular matrix material, wherein the bulk medium and the droplet medium are immiscible; which process comprises generating a plurality of droplets in the bulk medium, wherein each of said droplets comprises a droplet medium which comprises one or more biological cells and a natural extracellular matrix material, and arranging the droplets on the substrate in the bulk medium to form said plurality of elements spaced apart from one another, wherein each element comprises at least one of said droplets.

The invention also provides a nano-bioreactor comprising at least one droplet which comprises a droplet medium which comprises one or more biological cells and a natural extracellular matrix material, and at least one droplet of culture medium.

The invention also provides an array of nano-bioreactors comprising a plurality of nano-bioreactors spaced apart from one another on a substrate in a bulk medium, wherein each nano-bioreactor comprises at least one droplet which comprises a droplet medium which comprises one or more biological cells and a natural extracellular matrix material, and at least one droplet of culture medium, wherein the bulk medium and the droplet medium are immiscible, preferably wherein the bulk medium and the culture medium are immiscible.

### BRIEF DESCRIPTION OF THE FIGURES

FIG. 1 shows lipid bilayer supported droplet bio-printing for soft tissues. FIG. 1a, shows the setup for printing water-in-oil droplets containing cells in ECM. The printer consists of a high power piezo-driver for printing viscous materials, surface-modified glass printing nozzles and a temperature-controlled manipulator stage. The manipulator stage moves in accordance with the design under computer control and allows 3D positioning of droplets. Printed droplets are connected by adhesive DIBs to form patterned droplet networks. FIG. 1b shows the ejected droplet sizes can be controlled by printer nozzle sizes. Droplet diameters were tuned by changing printing parameters: the voltage and width of the printing pulse (n>3). Maximum and minimum droplet diameters are recorded in the plot. Example images show droplets containing Matrigel and fluorescently labelled HepG2 cells. FIG. 1c, Left: bright-field image of part of a printed droplet network. Droplets contain Matrigel and HepG2 cells (visible as small indentations) at 3×10⁷ mL⁻¹. Right: Texas-red-labelled lipids reveal DIBs in a printed network. FIG. 1d shows Matrigel droplet pairs containing dyes illustrate a stepped temperature increase approach to trigger DIB breakage and droplet connection. Top: DIB stays intact during a temperature increase from printing temperature (˜5° C.) to room temperature (RT) and is stable at RT for 2 h; middle: the DIB ruptures and the droplets fuse when the temperature is brought to 37° C.; bottom: the DIB breaks without droplet fusion after a stepwise temperature increase: from printing temperature to RT for 30 min, then RT to 37° C. for 2 h. FIG. 1e, Left, schematic of a 7×7×8 droplet network (8 layers of 7×7 droplets). Top right: illustration of the transfer of a printed tissue from printing oil to culture medium. Bottom right: images of a printed 7×7×8 droplet network (HepG2 cells (3×10⁷ mL⁻¹) in Matrigel) in oil and then transferred to culture medium (side view). Scale bars are 100 μm, except the labelled scale bars in FIG. 1b.

FIG. 2 shows printed neural tissues recapitulate cortical development events. FIG. 2a shows the timeline for the production of 3D neural tissues. Further details are described in Experimental section. FGF2, fibroblast growth factor; EGF, epidermal growth factor. FIG. 2b shows process formation in printed droplets during post-printing differentiation (ppd). Live cells were stained with Calcein AM (CAM). Example images of 1, 4, 8 and 14 days ppd show that printed NSCs project processes across droplets and formed more polarised morphology overtime. FIG. 2c shows 96±1% cell viability at day 1 post printing, n=5. FIG. 2d shows immunostaining reveals a neuronal (TUJ1⁺) signal in printed tissues that increases overtime. The lengths of the processes at several time points were quantified (n>15, P<0.001 between all three groups). Neural tissues in FIG. 2a-d were printed at a cell density of 2×10⁷ mL⁻¹ as 7×7×4 droplet networks. FIG. 2e, Left to right: schematic of a printed 7×7×8 droplet network; the corresponding bright-field image of a printed tissue constructed from cells at 3.5×10⁷ mL⁻¹; high-magnification CAM live-staining; DAPI (4′,6-diamidino-2-phenylindole) nuclear staining of a section from printed tissue at day 28 ppd. Arrows mark neural rosettes formed at 28 days ppd. FIG. 2f shows immunostaining for neural markers in rosettes: SOX2, CTIP2 and TUJ1. FIG. 2g shows longer differentiation leads to protuberance formation (arrow) and an increasing density of cortical neurons (CTIP2⁺). The bar chart shows the number of CTIP2⁺ cell per unit area of 100 μm×100 μm at 28 and 56 days ppd respectively (n=6, P<0.001). FIG. 2h shows mature neurons (MAP2⁺ and NeuN⁺) and astrocytes (GFAP⁺) also appeared after 56 days ppd. Ki67⁺ cells indicates pockets of sustained proliferation. The bar chart shows the number of GFAP⁺ cell per unit area of 200 μm×200 μm at day 56 ppd (n=6). Scale bars are 200 μm for the bright-field images in FIG. 2e and FIG. 2g, 50 μm for the last images of FIG. 2g and FIG. 2h and 100 μm elsewhere.

FIG. 3 shows functional and connected neural networks emerge from printed neural tissues. FIG. 3a shows fluo-4 calcium live imaging of printed neural tissues revealed spontaneous calcium oscillations at 4 days ppd. FIG. 3b shows single-cell traces are shown, colour-coded by their locations in regions of interest (ROI) as indicated in FIG. 3a. FIG. 3c shows network plots of correlation coefficients reveal limited communication between printed NSCs at 4 days ppd. FIG. 3d-f show spontaneous calcium oscillations (FIG. 3d), single-cell traces (FIG. 3e) and correlation coefficient analysis (FIG. 3f) of printed neural tissues at 44 days ppd. The data reveal increased neural communication after a longer period of differentiation. FIG. 3g shows fluorescence intensity map obtained from calcium living imaging at 65 days ppd after KCl (60 mM) treatment. Intensities are false-coloured according to the ‘fire’ Look Up Table of Fiji. FIG. 3h shows single-cell traces are shown colour-coded according to their locations in the ROIs indicated in FIG. 3g. FIG. 3i shows individual time-point frames of ROI1 (yellow outline) reveal that KCl-induced a rapid calcium transient in a cell. Images are false-coloured as in FIG. 3g. Scale bars are all 100 μm.

FIG. 4 shows pre-patterning reveals cell-autonomous process outgrowth and cell migration. FIG. 4a shows 3D-printed droplet network used for patterned cortical tissues: a 7×7×7 droplet network (orange) at the centre of a 13×13×13 droplet network (blue). FIG. 4b shows progression observed for tissues in which hNSCs are encased in Matrigel, as shown in FIG. 4a (hNSCs yellow, Matrigel blue). During the first 14 days of differentiation, hNSCs project processes into the Matrigel compartment. Cell migration followed. Migrated cells filled the Matrigel compartment at 28 days ppd. Astrogenesis occurred at 56 days ppd. Notably, the printed constructs adopted a more spherical geometry after weeks in culture (FIG. 12b). FIG. 4c shows printed fluorescently labelled hNSCs show cell migration. Top: Live images from RFP-hNSCs show the projection of processes and cell migration into the Matrigel compartments at 14 days ppd. The dashed lines indicate the boundaries of the inner and outer compartments and the dashed box shows the zoomed-in area of the following image. Bottom: The length of process outgrowth was quantified at 1, 3 and 14 days ppd (n=6 for each time points, P<0.005 between all three groups). The distance of cell migration was quantified at 3 and 14 days ppd (n=6 for each time points, P<0.005). FIG. 4d shows immunostaining reveals that printed tissues contain progenitors (SOX2⁺), young neurons (TUJ1⁺) and cortical neurons (CTIP2⁺) at 28 and 56 days ppd. FIG. 4e shows TBR1 staining reveals differentiation into deep layer cortical neurons at 56 days ppd. Also at this stage, printed constructs contain mature neurons (MAP2⁺), a small number of astrocytes (GFAP⁺) and proliferating cells (Ki67⁺). FIG. 4f shows progression observed for hNSCs encased in hAs, as shown in FIG. 4a (hNSCs yellow, hAs blue). hNSCs project processes into the hAs compartment (14 days ppd). Cell migration follows (28 days ppd). hAs remained at the periphery of the tissues. The cell densities during the printing, NSCs (3.5×10⁷ mL⁻¹) and hAs (1×10⁷ mL⁻¹), mimicked early-stage astrogenesis. FIG. 4g shows CAM live-staining image of printed tissues reveals processes that cross from the hNSCs compartment to the hAs compartment. FIG. 4h shows immunostaining reveals the cellular architecture of 3D tissues. The surface contains evenly distributed neurons (TUJ1⁺) and astrocytes (GFAP⁺). The section through a central plane shows neurons within a shell of astrocytes. DAPI and TUJ1 staining revealed that hNSCs have migrated towards the astrocyte compartment. Scale bars are 200 μm in FIG. 4g (left image), and FIG. 4h (first two images) and 100 μm in the rest of the images.

FIG. 5 shows astrocytes induce axonal fasciculation in pre-patterned cortical tissues. FIG. 5a, Left: 2D cross-section showing the pre-patterned hNSCs (in)-Matrigel (out) constructs at 28 days ppd. Right: confocal z-projection images reveal that both progenitors (SOX2⁺) and young neurons (TUJ1⁺) are on the surface of the structures at 28 days ppd. The dashed box shows the zoomed-in area in the following image, in which the dashed line indicates the site of the fluorescence profile in FIG. 5d (control). FIG. 5b, Left: 2D cross-section showing the pre-patterned hNSCs (in)-hAs (out) construct at 28 days ppd. Right: confocal z-projection images reveal the existence of astrocytes (GFAP⁺), neurons (TUJ1⁺) and neural progenitors (SOX2⁺) on the tissue surface. The dashed box indicates the zoomed-in area shown in the following image. Importantly, process bundles were observed in FIG. 5b but not in FIG. 5a, indicating astrocyte-assisted neural fasciculation. The dashed line indicates the site of the fluorescence profile in FIG. 5d (through the process bundle). FIG. 5c shows process densities are similar for both constructs (FIG. 5a and FIG. 5b). Process density is calculated as the ratio of the TUJ1⁺-fluorescence area to the total area (n=6, P>0.9, NS: not significant). FIG. 5d shows profile plots of fluorescence intensity along the white dashed lines indicated in FIG. 5a and FIG. 5b. The black dashed box shows the position of a neural process bundle. Whisker plot indicates the width of bundles in printed cortical tissues FIG. 5b compared to FIG. 5a (control, n>7, P<0.001). Each point indicates the width of one bundle (in FIG. 5b) or process (in FIG. 5a). FIG. 5e, Left: 2D cross-section of the design, hAs (left)-hNSCs (right). Right: confocal z-projection images reveal neural progenitors (SOX2⁺) migrating along neural process bundles. The dashed box shows the zoomed-in area for the following images. Dashed lines indicate the sites for the fluorescence profile in FIG. 5f (Left, control and right, process bundles1-4). FIG. 5f shows Left: fluorescence profiles show that process bundles exist in the hAs compartment (left) but not in the hNSCs compartment (right). Right: Whisker plot comparing the width of process bundles in the hAs compartment with the control at the hNSCs compartment in FIG. 5e (n>7, P<0.001). FIG. 5g shows the alignment of migrating cells with process bundles. Left: example of a measurement of the angle between a migrating cell and a process bundle. Right: bar chart shows that a majority of cells subtend an angle of <200 with an associated process bundle (n=68 cells). Scale bars are 200 μm in FIG. 5a (first two images), FIG. 5b (first two images) and FIG. 5e (first image), 20 μm in FIG. 5g and 100 μm in the rest of the images.

FIG. 6 shows fast production of differentiated cortical tissues. FIG. 6a shows the timeline for the production of 3D differentiated cortical tissues. iPSC-derived human cortical neurons (hCNs) were used for bio-ink without pre-culture, whereas hAs were harvested from 2D culture at day 4-10. Further details are described in Experimental section. FIG. 6b shows immunostaining of tissues constructed from hCNs (TUJ1⁺ and vGlut1⁺) and hAs (GFAP⁺) at day 13 post printing. FIG. 6c shows a schematic of patterned and reverse-patterned cortical tissues and the observed progression. FIG. 6d, Top: immunostaining of tissue sections (surface and a central plane) at 13 days ppd indicates a thicker astrocyte shell compared to the astrocyte shell of the tissue in FIG. 4h (same pattern but with hNSCs). hCNs have invaded the astrocyte compartment, while most hAs remain in the outer compartment with a small fraction of hAs having migrated towards the centre. Bottom: immunostaining of tissue sections (surface and a central plane) from the reverse-patterned cortical tissue at 14 days ppd. The arrow indicates an astrocyte that has migrated into the exterior neuron compartment. Synaptic staining (vGlut1) confirmed the mature differentiated stage of the hCNs. FIG. 6e, Left: example of segmentation used to define the inner (blue) and outer (orange) compartments that are used for the stacked bar chart (right). The two compartments have equal area. Right: Stacked bar chart of hAs distribution for the two different patterns. Tissues were constructed with hCNs (red, 3.5×10⁷ mL⁻¹) and hAs (green, 3.5×10⁷ mL⁻¹). Scale bars are 200 μm in FIG. 6b (first image), FIG. 6d (first two images of each row) and 100 μm in the rest of the images.

FIG. 7 shows the results for printing arrays of picoliter and nanolitre droplets with cells in ECM. FIG. 7a shows the printer setup for printing droplet arrays. Droplets were ejected through printer nozzles under the control of computer designed electric signals. Printing sample was loaded into the printing nozzle and separated by an oil plug from the water, which transduced the propelling force induced by the electric signals. Low microliters of bio-ink were needed with this setup. Printing oil bath consisted of a mixture of silicon oil and undecane. Printing container was placed on a temperature-controlled stage and XYZ manipulator stage. Printed droplets contain both cells and ECM and are from low picoliter to nanolitre in volume. FIG. 7b shows an example of printed droplet array containing MDA cells (1×10⁷/mL) in Matrigel. The printed droplets are ˜120 μm in diameter. FIG. 7c shows two examples of printed droplet arrays containing two cell types in collagen. The same bio-ink was used to produce droplets with a diameter of either ˜20 (top) or ˜60 (bottom) μm. Bio-ink: HepG2 cells with green (8×10⁶/mL) and far-red (4×10⁶/mL, false coloured as magenta) cell tracker in collagen. FIG. 7d shows the size distribution of printed droplets with different sizes. Scale bars are: 200 μm in FIG. 7b and 50 μm in FIG. 7c, except the top right image, which is 20 μm.

FIG. 8 shows the results for the production of patterned droplet arrays. FIG. 8a shows a schematic of sequential printing to generate top-bottom patterned droplet arrays. First droplet (green) was print at 5° C. and immediately formed contact angle with the printing substrate. The droplet was then incubation at 25° C. for 10 mins for partial gelation before the temperature was cooled back to 5° C. The second droplet was printed on top of the first partially gelated (sticky) droplet to produce the desired top-bottom pattern. Patterned droplets were then incubated at 37° C. for 60 mins. Notably, the first droplet formed contact angle with both substrate and the second droplet. FIG. 8b shows confocal fluorescent images of top-bottom patterned droplet arrays. Left: an example of the top-bottom patterned droplet pair with bottom image (top left), reconstructed 3D image through z-stack (top right) and images at different confocal planes (bottom three images). Right: Constructed 3D image of an array of top-bottom patterned droplet pairs. FIG. 8c shows constructed left-right patterned droplet pairs. Left: schematic of left-right patterned droplet pair from XY dimensions with a bottom view (top) and images of the patterned droplet pair (bottom). The dashed line indicates the location of the fluorescence prolife in the chart (right). Middle: schematic of left-right patterned droplet pair from XZ dimensions (top) and the 3D reconstructed image. Right, profile diagram of both green and red fluorescence intensity along the dashed line (left). Sharp droplet boundary is observed in the centre of the diagram. FIG. 8d shows patterned top-bottom droplet pair with different sizes. From left to right: schematic of the pattern from XY dimensions with a bottom view, fluorescence image of the bottom view, schematic of the pattern from XZ dimensions, reconstructed 3D fluorescent image, and profile diagram of both green and red fluorescence intensity along the dashed line (second image). FIG. 8e shows patterned three-droplet assemblies. Top row: patterned left-middle-right droplet assembly with different sizes. Bottom row: patterned bottom-middle-top droplet assembly with different sizes. For both patterns, from left to right: schematic of the pattern from XY dimensions with a bottom view, fluorescence image of the bottom view, schematic of the pattern from XZ dimensions, and reconstructed 3D fluorescent image. Scale bars are all 100 μm.

FIG. 9 shows the results for printed cortical astrocyte arrays for functional assays. FIG. 9a, Printed human primary cortical astrocyte array. Astrocytes were printed at a cell density of 4×10⁷/mL in Matrigel. Left, Calcein AM (CAM) staining of printed astrocytes at 2 days post printing. The dashed box indicates the area of the higher magnification image on the right (top two images in the middle). Top right row: higher magnification images of astrocyte printed in Matrigel (CAM staining image and the corresponding brightfield image) and image of astrocytes printed in agarose as the matrix. Bottom right row: immunostaining images of printed astrocytes with astrocyte markers vimentin (yellow) and GFAP (magenta). FIG. 9b shows spontaneous calcium fluctuations of astrocytes in printed droplet arrays. Left: Individual time-point frames of Fluo-4 calcium live imaging. Right: single-cell traces of the cells indicated by the colour-coded arrows in the images (left). FIG. 9c shows patterned droplet pairs with different droplet interface areas. Droplets contained either astrocytes (CAM stained, green) or metastatic cancer cells, MDA (RFP labelled, Red). Top row: left-right patterned. bottom row: top-bottom patterned. For both patterns from left to right: schematic of the patterned droplet pair from XY dimensions with a bottom view, schematic of the pattern from XZ dimensions, and the fluorescent image at 2 days post printing. Right, schematic of top-bottom patterned droplet pair and the fluorescent image (bottom view) at 2 days post printing. Notably, the top-bottom patterned droplet pair has a lager droplet contacting area compared to the left-right patterned droplet air, presumably due to wetting effect with the printing substrate and gravity. FIG. 9d shows scatter plots of the proportions of astrocytes with spontaneous calcium fluctuations (left) and the intracellular calcium concentrations (indicated by the fluorescence intensity of Fluo-4) of astrocytes at different conditions: 2D monolayer cell culture, printed single astrocyte droplet arrays, printed left-right patterned (astrocyte left, MDA right) droplet arrays and printed top-bottom patterned (astrocyte bottom, MDA top) droplet arrays. n≥20 for all conditions. *: P<0.05, **: P<0.01, ***: P<0.001 and ****: P<0.0001. Scale bars are all 100 μm in FIG. 9a and 200 μm in FIG. 9c.

FIG. 10 shows droplet printing and oil to medium transfer of printed droplet networks. FIG. 10a shows the printing voltage required to eject droplets containing different materials. The minimum voltage required for droplet ejection is reported as a multiple of that required for the ejection of water droplets (˜18 V). Data points in each group were generated with different printing nozzles. Chitosan: 2.5% (w/v); Matrigel: undiluted; collagen: 3.6 mg mL⁻¹. FIG. 10b shows phase transfer of a droplet pair from oil (left) to culture medium (right). Cells in one droplet were stained with Calcein AM (CAM, live cell staining). The images show that the droplets did not exchange their contents (cells) during phase transfer. Droplets containing: 3.0×10⁷ mL⁻¹ HepG2 cells in Matrigel. FIG. 10c shows lipid diffusion during post-printing culture. The fluorescence of the DIBs (labelled with fluorescent lipids) disappeared from the printed tissue after 2 days of culture (no obvious fluorescence change after 2 hours of transfer into medium), indicating lipid diffusion. Droplets contained: 3.0×10⁷ mL⁻¹ HepG2 cells in Matrigel. Scale bars are 200 μm in b and 100 μm in c.

FIG. 11 shows live/dead staining of printed neural tissue at day 1 post printing. Neural tissues were printed with 2.0×10⁷ mL⁻¹ hNSCs in Matrigel. CAM (live, green) and propidium iodide (dead, red) staining were conducted at 1 day post printing. Scale bars are 100 μm.

FIG. 12 shows printed viable hNSCs at a lower cell density. FIG. 12a shows neural process outgrowth in printed neural tissues. Process outgrowth and branch number of printed NSCs were measured at day 1 and day 4 post-printing culture respectively. Total outgrowth: processes emanating from both cell bodies and other processes; branches: processes derived from other processes only. Over 50 cells were analysed at each time point. FIG. 12b shows printed neural tissues in differentiation culture. CAM staining of printed neural networks at 4, 8 and 14 days post-printing differentiation. FIG. 12c shows SOX2 and TUJ1 immunostaining of day 14 neural tissue. All neural tissues were printed as 7×7×4 droplet networks with 2.0×10⁷ mL⁻¹ hNSCs in Matrigel. Scale bars are 200 μm in FIG. 12b and the first image of FIG. 12c, and the rest are 100 μm.

FIG. 13 shows printed neural tissue at 100 days post-printing differentiation. CAM and TUJ1 staining demonstrates that printed tissues are viable for long-term culture. The tissue was printed as a 7×7×8 droplet network with 3.5×10⁷ mL⁻¹ hNSCs (Axol, ax0018) in Matrigel. Scale bars are 100 μm.

FIG. 14 shows higher magnification images of FIG. 4e. FIG. 14a shows CAM fluorescent image of the whole printed tissue (hNSCs (in)-hAs (out)). FIG. 14b shows higher magnification image of hNSCs in the inner compartment of a. FIG. 14c shows high magnification image of the outer compartment of a. FIG. 14d shows higher magnification image of neural projections in c. FIG. 14e shows higher magnification images of hAs in the outer compartment of c (left: CAM; right, GFAP immunostaining). Scale bars are 200 μm in FIG. 14a, 50 μm in FIG. 14b, 100 μm in FIG. 14c, 20 μm in FIG. 14e.

FIG. 15 shows tissue section of pre-patterned hAs (left)-hNSCs (right) construct. FIG. 15a shows a schematic of the printed tissue pattern (as shown in FIG. 5e). FIG. 15b shows bright-field image of the printed tissue at 28 days. FIG. 15c shows immunostaining of a centre section slice revealed that progenitors (SOX2⁺) and neurons (CTIP2⁺, TUJ1⁺) exist in the hAs compartment at 28 days post-printing differentiation, indicating hNSC migration.

FIG. 16 shows immunostaining of printed cortical tissue. Positive synaptophysin and MAP2 staining show differentiated neurons at 14 days post-printing culture. Scale bars: 200 μm.

FIG. 17 shows the toxicity test of DPhPC on hNSCs. RFP-hNSCs were cultured in neural maintenance medium containing no lipid (control) or DPhPC at either 100 μg/mL or 400 μg/mL. Arrows indicate the formation of neural rosette-like structures under all conditions after three days of culture. No obvious differences were seen in the presence of DPhPC.

FIG. 18 shows a full circuit diagram of the piezo-driver used in Example 1.

FIG. 19 shows functional and miniature neural tissues from printed arrays. FIG. 19a, human primary cortical astrocyte printed in an array. Astrocytes were printed at a cell density of 4×10⁷ mL⁻¹ in Matrigel. Left: Calcein AM (CAM) staining of printed astrocytes at 2 days post printing. The dashed box indicates the area of the higher magnification image on the right (top two images in the middle). Top right row: higher magnification images of astrocyte printed in Matrigel (CAM staining image and the corresponding brightfield image) and image of astrocytes (brightfield with CAM staining) printed in agarose as the matrix. Bottom right row: immunostaining images of printed astrocytes with astrocyte markers vimentin (yellow) and GFAP (magenta). FIG. 19b, Spontaneous calcium fluctuations of astrocytes in printed droplet arrays. Left: Individual time-point frames of Fluo-4 calcium live imaging. Right: single-cell traces of the cells indicated by the colour-coded circles in the images (left panel). FIG. 19c, Neural differentiation process from iPSCs to NPCs. iPSCs were cultured with growth factors, FGF and TGF-β, to maintain the pluripotency of the stem cells. Neural induction were induced with two SMAD inhibitors, SB431542 and LDN193189, over 7 days. Further neural differentiation were conducted with neural maintenance medium containing N2 and B27. FIG. 19d, Spontaneous calcium fluctuation of astrocytes when they were co-cultured with RFP-NPCs. Left: an example droplet containing RFP-NPCs at day 2 post printing. Middle: an example droplet containing astrocytes and RFP-NPCs (3:1 cell density ratio and 4×10⁷ mL⁻¹ total cell density). Right: single-cell trace of the cell indicated by the circle in the image. FIG. 19e, Scatter plots demonstrated the differences in cell function of astrocytes under different cell microenvironment. Proportions of astrocytes with spontaneous calcium fluctuations were measured at four conditions: 2D monolayer cell culture, and printed astrocyte, astrocyte-NPC, and astrocyte-MDA (a metastatic breast cancer cell line) droplet arrays. n>20 for all four conditions. *: P<0.05, **: P<0.01, ***: P<0.001 and ****: P<0.0001. FIG. 19f, Astrocytes in droplet arrays were responsive to KCl (60 mM) stimulation. Left two images: two time-point (t1 and t2) frames of Fluo-4 calcium live imaging of neural cells response to KCl stimulation. Right: single-cell traces of the cells indicated by the colour-coded circles in the images (left panel) after KCl stimulation. Scale bars are all 100 μm in FIG. 19a and 150 μm in FIG. 19d.

FIG. 20 shows the results for neurotoxicity testing using printed neural tissue arrays. FIG. 20a, The experimental process of drug testing using printed neural tissue arrays. From left to right: 2D cultured astrocytes and RFP-NPCs were harvested and added to Matrigel for the preparation of bio-ink with a cell density ratio of 3:1 (astrocytes vs RFP-NPCs) and total cell density of 4×10⁷ mL⁻¹. The prepared bio-ink were printed into droplet arrays with individual droplet size around 100 μm. On the next day, cancer chemotherapy drugs were added to the culture medium. The neurotoxicity of the drugs were assessed after a further 24 hrs. FIG. 20b, Fluorescent images of neural tissues treated with DMSO (control) and cancer chemotherapy drugs with different reported neurotoxicity risks: 5-Fluorouracil (minor), Carboplatin (moderate), Taxol (high). Neurotoxicity against neural progenitor cells (RFP-NPCs in red) was observed. FIG. 20c, Comparison of the neurotoxicity of different chemotherapy drugs. Consistent with clinic observations, Taxol and Carboplatin showed higher toxicity against neural cells compared to 5-Fluorouracil. n>25, P<0.05.

FIG. 21 shows neural migration in printed nano-bioreactors. FIG. 21a, A sequential printing process for the production of nano-bioreactors. Left to right: pL to nL droplets, containing cells and ECM, were printed in glyceryl trioleate, an optimised oil condition; after gelation at 37° C. for 10 mins, defined culture medium was printed to encapsulate the tissue; biological events were then assessed in the nano-bioreactors. FIG. 21b, Schematic (left) and image (right) show the printed nano-bioreactors with defined sizes. FIG. 21c, Schematic of neural migration observed in nano-bioreactors with patterned tissues where RFP-NPCs were encapsulated in ECM. Firstly, the neural cells (RFP-NPCs) projected processes into the ECM compartment, followed by cell migration. FIG. 21d, Neural migration at day 1, 4, and 6 in nano-bioreactor. Dashed boxes indicate the regions with the higher magnification images in the bottom row. Dashed lines in the bottom row images show the boundary of the NPC and ECM compartments. The white arrows indicating the migrating cells along neural processes. FIG. 21e, Quantification of neural migration in the printed nano-bioreactors. Increasing migration distance was observed over the first 4 days post printing. n=13, P<0.05. Scale bars are 200 μm and 100 μm in the top and bottom rows of FIG. 21d respectively.

FIG. 22 shows the results from printing droplets with defined number of cells in ECM. Top row, from left to right: printed droplets containing 1, 2, 3, 4 and 5 cells respectively. Droplets are around 75 μm in diameter. Bottom graph shows the distribution of cell numbers in droplets at different cell density of the bio-ink: 0.25, 0.5, 1 and 2.5 million/mL. ˜40% of the droplets at 1 million/mL containing single cell. This could be combined with a droplet sorter to achieve printing with cell number defined droplets.

FIG. 23 shows examples from the oil toxicity test using iPSCs derived NPCs. 2D cultured NPCs were added with oil on top of medium and cultured for 5 days. Life (CAM, in green) and Dead (PI, in read) staining were conducted on day 5. No obvious toxicity was observed with sunflower oil.

FIG. 24 shows the results from printing multiple droplets containing different cells in nano-bioreactors. Printed three droplets in one nano-bioreactor: two droplets containing RFP—NPCs and the third droplets containing astrocytes (no colour). This could be used for the investigation of secretory effects between different cell types at nanoliter scale.

### DETAILED DESCRIPTION

Apparatus

The present invention relates to an apparatus for producing a droplet assembly, which apparatus comprises: at least one droplet generator suitable for generating droplets of a viscous droplet medium; a droplet receiving region which is moveable relative to the at least one droplet generator; a temperature controller; and a control unit, which control unit is adapted to control the dispensing of droplets from the at least one droplet generator and the movement of the droplet receiving region relative to the at least one droplet generator, wherein the apparatus is adapted to produce a droplet assembly in the droplet receiving region, wherein the droplet assembly comprises a plurality of droplets, wherein each of said droplets comprises a droplet medium.

Usually, the apparatus is adapted to produce a droplet assembly in the droplet receiving region, wherein the droplet assembly comprises a plurality of droplets, wherein each of said droplets comprises (i) the droplet medium, and (ii) an outer layer of amphipathic molecules around the surface of the droplet medium. Alternatively, the apparatus may be adapted to produce a droplet assembly in the droplet receiving region, wherein the droplet assembly comprises a plurality of droplets, wherein each of said droplets consists of the droplet medium, without any amphipathic molecules.

Droplet Medium

Usually, the at least one droplet generator is suitable for generating droplets of a droplet medium which has viscosity, measured at the temperature of the droplet medium in the droplet generator at the time of generating a droplet thereof, of at least 50 mPa·s, preferably at least 75 mPa·s, more preferably 100 mPa·s. For instance, the at least one droplet generator is typically suitable for generating droplets of a droplet medium which has viscosity, measured at the temperature of the droplet medium in the droplet generator at the time of generating a droplet thereof, of from 100 mPa·s to 10 Pa·s, for instance from 100 mPa·s to 5 Pa·s or from 100 mPa·s to 1 Pa·s.

Typically, for instance when the apparatus is in use, or is prepared for use, the droplet generator contains said droplet medium.

Typically, the droplet medium has a viscosity, measured at a temperature of between −5 and 15° C., for instance between 0 and 10° C., preferably at about 5° C., of at least 50 mPa·s. For instance, the droplet medium may have a viscosity, measured at a temperature of between −5 and 15° C., for instance between 0 and 10° C., preferably at about 5° C., of at least 75 mPa·s. The droplet medium may have a viscosity, measured at a temperature of between −5 and 15° C., for instance between 0 and 10° C., preferably at about 5° C., of at least 100 mPa·s. Typically, the droplet medium has a viscosity, measured at a temperature of between −5 and 15° C., for instance between 0 and 10° C., preferably at about 5° C. of from 100 mPa·s to 10 Pa·s, for instance from 100 mPa·s to 5 Pa·s or from 100 mPa·s to 1 Pa·s.

Typically, the droplet medium is an aqueous droplet medium, i.e. the droplet medium typically comprises water. The aqueous droplet medium may be any suitable aqueous droplet medium, for instance, the aqueous droplet medium may comprise a pre-gel solution of a hydrogel.

The droplet medium may be capable of gelation in response to a stimulus. Typically, gelation is induced by a change in temperature, for instance heating or cooling the droplet medium from the temperature at the time of generating a droplet. Preferably, the droplet medium is capable of gelation on heating.

Typically, the droplet medium comprises a natural extracellular matrix material. Thus, typically, the droplet medium comprises a natural extracellular matrix that is biological in origin. The droplet medium may comprise at least one selected from collagen, fibronectin, hyaluronic acid and laminin. For instance, the droplet medium may comprise collagen, for instance collagen derived from animals. Droplet media comprising biologically-derived matrix materials may be particularly suitable for forming gel networks which support biological cells. The natural extracellular material may comprise a material which approximates the properties of the basement membrane matrix. Preferably, the natural extracellular matrix material is matrigel. Matrigel is the trade name for a gelatinous protein mixture produced by Corning Life Sciences secreted by Engelbreth-Holm-Swarm mouse sarcoma cells. For instance, the droplet medium may comprise matrigel supplemented with one or more additives, typically where the additives are selected from collagen, fibronectin, hyaluronic acid and laminin.

Typically, at least 80% by volume of the droplet medium is a natural extracellular matrix material, preferably matrigel, for instance at least 90% by volume of the droplet medium may be a natural extracellular matrix material, preferably matrigel. For example, at least 95% by volume of the droplet medium may be a natural extracellular matrix material, preferably matrigel. Typically, the droplet medium comprises undiluted natural extracellular matrix material, preferably undiluted matrigel.

The droplet medium may comprise any natural occurring substance, for instance any natural occurring salt, protein, fat, lipid or other molecule. The droplet medium may comprise collagen, fibronectin, hyaluronic acid and/or laminin.

Typically, the droplet medium comprises biological cells. Typically, the droplet medium comprises biological cells at a density of at least 10⁴ cells/mL typically at least 10⁷ cells/mL preferably at least 10⁶ cells/mL. Typically, the droplet medium comprises biological cells at a density of up to 10⁷ cells/mL, for instance of up to 7.5×10⁷ cells/mL or up to 5×10⁷ cells/mL. For instance, the droplet medium may comprise biological cells at a density of from 1×10⁶ cells/mL to 7.5×10⁷ cells/mL, or from 1×10⁶ cells/mL to 5×10⁷ cells/mL, or from 1×10⁶ cells/mL to 4×10⁷ cells/mL.

The cells may be any biological cells, for instance cells originating from any plant, animal, fungus or single-celled organism. Typically, the cells are mammalian cells, preferably human cells. For instance, the biological cells may be human liver cells, ovarian cells, breast cells or brain cells. The biological cells may be cancer cells. Typically the biological cells are selected from cell types found in soft tissue, for instance brain cells. Thus, the biological cells may comprise brain cells, typically human brain cells. The brain cells may comprise neural stem cells, neural progenitor cells, neurons, astrocytes, microglia or endothelial cells. Preferably, the brain cells comprise neural stem cells.

Typically, the droplet medium comprises a natural extracellular matrix material and biological cells, for instance cells as defined above. Preferably, the droplet medium comprises a natural extracellular matrix material and mammalian cells, for instance a natural extracellular matrix material and brain cells, typically a natural extracellular matrix material and human brain cells. The natural extracellular matrix material is preferably matrigel. For instance, the droplet medium may comprise matrigel and biological cells, preferably matrigel and mammalian cells, for instance matrigel and brain cells, typically matrigel and human brain cells. Typically, the natural extracellular matrix material is undiluted. The droplet medium may therefore comprise an undiluted natural extracellular matrix material and biological cells. Preferably the droplet medium comprises undiluted matrigel and biological cells.

Typically, the natural extracellular matrix material and the biological cells together form at least 80% by volume of the droplet medium, preferably at least 90% by volume of the droplet medium, more preferably at least 95% by volume of the droplet medium, for instance at least 99% by volume of the droplet medium. Typically, the droplet medium consists of the natural extracellular matrix material and the biological cells, preferably wherein the natural extracellular matrix material is matrigel.

Droplet Receiving Region

Typically, the droplet receiving region is a container. Typically, the container contains the bulk medium. Alternatively, the droplet receiving region may be a substrate, or platform. The bulk medium may be disposed on the substrate or platform. Alternatively, the droplet receiving region may be a substrate wherein the substrate is placed within the bulk medium.

The container of the apparatus of the invention may be any suitable container. Typically, the container comprises a polymer, such as poly(methyl methacrylate). For instance, the container may comprise a well micromachined from said polymer. For instance, the bottom surface of the container comprises a polymer, such as poly(methyl methacrylate). Often, the bottom surface of the container comprises glass. The apparatus may be configured to dispense at least one droplet which is in contact with the glass. The glass may prevent the droplet from moving around.

The substrate of the apparatus of the invention may be any suitable substrate. Typically, the substrate comprises a polymer, such as poly(methyl methacrylate) or polystyrene. The substrate may comprise glass. The apparatus may be configured to dispense at least one droplet which is in contact with the glass. The glass prevents the droplet from moving around. Typically, the substrate can be removed from the apparatus after printing and transferred to a container, for instance a container containing cell culture medium. The substrate may comprise a plurality of microwells.

Bulk (Hydrophobic) Medium

Typically, the droplet receiving region further comprises a bulk medium, wherein the bulk medium and the droplet medium are immiscible. The bulk medium is preferably a hydrophobic medium. Thus, when the droplet medium is an aqueous medium, the bulk medium is preferably a hydrophobic medium. Alternatively, however, the droplet medium may be a hydrophobic medium, and the bulk medium may be an aqueous medium. Preferably, the droplet medium is an aqueous medium and the bulk medium is a hydrophobic medium.

When the droplet receiving region is a container, the container may contain the bulk medium, wherein the bulk medium and the droplet medium are immiscible. When the droplet receiving region is a container, a substrate may be immersed in the bulk medium, wherein the bulk medium and the droplet medium are immiscible. Typically, the droplet medium is an aqueous medium and the container of the apparatus of the invention contains a hydrophobic medium.

The hydrophobic medium may be selected from a wide range of materials. The hydrophobic medium may comprise a single hydrophobic compound. Alternatively, it may comprise a mixture of two or more different hydrophobic compounds. The hydrophobic medium can, for instance, be selected to affect the buoyancy of the droplet and the speed of formation of the layer of amphipathic molecules (if present) around at least part of the droplet after the droplet is first introduced into the hydrophobic medium. The hydrophobic medium is typically an oil. The oil may be a single, pure, compound, or the oil may comprise a mixture of two or more compounds. It is usually desirable that the oil does not significantly destabilize any bilayers of amphipathic molecules, where present, in the droplet assembly.

The oil may for instance comprise silicone oil (for instance poly phenyl methyl siloxane). Silicone oil is advantageous on account of its density being close to that of water, which ensures that the droplet is approximately neutrally buoyant in water. The silicone oil may for instance be poly phenyl methyl siloxane, which has a density of about 1 g cm⁻³. The oil may therefore comprise a single silicone oil, for instance poly phenyl methyl siloxane. Alternatively, the oil may comprise a mixture of two or more different silicone oils. Any suitable silicone oil may be used. For instance, the oil may comprise silicon oil DC200 (a polymer comprising monomer units of —O—Si(CH3)2-), poly(dimethylsiloxane) (PDMS), hydroxy terminated, or PDMS 200. In some embodiments, the silicone oil is a poly(methylphenylsiloxane), such as AR20.

Additionally or alternatively, the oil may comprise a hydrocarbon. When the oil comprises a hydrocarbon it may comprise a single hydrocarbon compound, or a mixture of two or more hydrocarbons.

Additionally or alternatively, the bulk medium may comprise or consist of a naturally occurring oil, for instance sunflower oil. The bulk medium may comprise or consist of the primary constituent of naturally occurring oil, for instance, the bulk medium may comprise or consist of glyceryl trioleate and/or glyceryl trilinoleate.

The oil may comprise a solid. A solid hydrocarbon may, for instance, be used in combination with a silicone oil. The oil may, for instance, be a mixture of solids that dissolve to form a liquid.

When the oil comprises a hydrocarbon, the hydrocarbon may be branched or unbranched, for example a hydrocarbon having from 5 to 40 carbon atoms, or from 5 to 30 carbon atoms (although hydrocarbons of lower molecular weight would require control of evaporation). Preferably, the hydrocarbon is a liquid at the printing temperature of the droplet used in the invention. Suitable examples include alkanes or alkenes, such as hexadecane, decane, pentane or squalene. Usually, the oil comprises a hydrocarbon.

Typically the hydrocarbon is an unsubstituted C₅-C₂₀ alkane, typically a C₈-C₁₅ alkane, for instance undecane. Shorter alkanes may be suitable, for instance, in assemblies for which buoyancy effects are less important and whose outer layer of amphipathic molecules (if present), on at least part of the surface of the droplet, may form more quickly.

In some embodiments the hydrocarbon is a longer-chain hydrocarbon, such as unsubstituted C₁₅-C₄₀ alkane. For instance, an unsubstituted C₁₆-C₃₀ alkane chain, such as squalene.

Typically, the hydrophobic medium comprises silicone oil and a hydrocarbon. Typically, the hydrocarbon is a straight-chained, unsubstituted C₅-C₂₀ alkane, typically a straight-chained, unsubstituted C₈₋₁₅ alkane, preferably the hydrocarbon is undecane. Typically, the hydrophobic medium comprises said silicone oil and said hydrocarbon in a ratio by volume of from 5:1 to 1:5, preferably from 2:1 to 1:5, and more preferably 1:1 to 1:4, for instance 1:2 to 1:4. For instance, the hydrophobic medium may comprise said silicone oil and undecane in a ratio by volume of from 5:1 to 1:5, preferably from 2:1 to 1:5, and more preferably 1:1 to 1:4, for instance 1:2 to 1:4. The hydrophobic medium may comprise silicone oil and undecane in a ratio by volume of about 1:1. The hydrophobic medium may comprise silicone oil and undecane in a ratio by volume of about 1:2. The hydrophobic medium may comprise silicone oil and undecane in a ratio by volume of about 1:3. The hydrophobic medium may comprise silicone oil and undecane in a ratio by volume of about 1:4.

Preferably, the ratio by volume of the hydrocarbon and the silicone oil is selected such that the density of the hydrophobic medium matches that of the droplet medium. Thus, typically the droplet medium comprises a natural extracellular matrix material as described herein and the hydrophobic medium comprises said silicone oil and said hydrocarbon in amounts that match the density of the natural extracellular matrix material at the printing temperature.

For instance, the droplet medium may comprise a natural extracellular matrix material as described herein and the hydrophobic medium comprises said silicone oil and said hydrocarbon in a ratio by volume of from 5:1 to 1:5, preferably from 2:1 to 1:5, and more preferably 1:1 to 1:4, for instance 1:2 to 1:4. Preferably the droplet medium may comprise a natural extracellular matrix material as described herein, typically matrigel, and the hydrophobic medium comprises a silicone oil and undecane in ratio by volume of from 5:1 to 1:5, preferably from 2:1 to 1:5, and more preferably 1:1 to 1:4, for instance 1:2 to 1:4.

Amphipathic Molecules

As mentioned above, the apparatus may be adapted to produce a droplet assembly in the droplet receiving region, wherein the droplet assembly comprises a plurality of droplets, wherein each of said droplets comprises (i) the droplet medium, and (ii) an outer layer of amphipathic molecules around the surface of the droplet medium. When amphipathic molecules are present, typically, the droplet receiving region further comprises the bulk medium and amphipathic molecules. Typically, the droplet receiving region is a container and when amphipathic molecules are present, typically, the container contains the bulk medium and amphipathic molecules.

Typically, the concentration of amphipathic molecules in the bulk medium is less than or equal to 15 mg mL⁻¹. For instance, the concentration of amphipathic molecules may be from 0 to 10 mg mL⁻¹. Usually, the concentration of amphipathic molecules is from 0.05 mg mL⁻¹ to 10 mg mL⁻¹, for instance, from 0.5 mg mL⁻¹ to 10 mg mL⁻¹. Typically, the droplet medium is an aqueous medium and the droplet is disposed in a hydrophobic medium and the concentration of amphipathic molecules is the concentration of amphipathic molecules in the hydrophobic medium.

Amphipathic molecules are molecules which have both hydrophobic and hydrophilic groups. The outer layer of amphipathic molecules usually comprises a monolayer of amphipathic molecules on the surface of the droplet. The monolayer is typically formed and maintained naturally by the interaction of the hydrophilic and hydrophobic groups with the aqueous medium and the bulk medium so that the molecules align on the surface of the droplet with the hydrophilic groups facing inwards towards the aqueous medium and the hydrophobic groups facing outwards, for instance towards a hydrophobic medium.

The amphipathic molecules may, for instance, comprise non-polymeric amphipathic molecules, for instance lipids, e.g. phospholipids. Alternatively, the amphipathic molecules may comprise polymeric amphipathic molecules, for instance triblock co-polymers.

An important class of amphipathic molecules which can be used in the droplet assembly is lipid molecules. The lipid molecules may be any of the major classes of lipid, including phospholipids, fatty acids, fatty acyls, glycerolipids, glycerophospholipids, sphingolipids, sterol lipids, prenol lipids, saccharolipids and polyketides. Some important examples include phospholipids and fatty acids, for instance phospholipids. The lipid molecules may be naturally occurring or synthetic. Whilst the formation of a bilayer from lipid molecules has been demonstrated the method is expected to be appropriate for any amphipathic molecules.

Accordingly, the amphipathic molecules that form an outer layer on at least part of the surface of the aqueous medium typically comprise phospholipid molecules. The phospholipid molecules may be the same or different, i.e. the amphipathic molecules comprise a single kind of phospholipid, or a mixture of two or more different phospholipids. Phospholipids are well known to the skilled person and many are commercially available, from suppliers such as Avanti Polar Lipids. The phospholipid molecules may be glycerophospholipids or phosphosphingolipids or a mixture of the two. The phospholipid molecules may comprise anionic phospholipids, phospholipids comprising primary amines, choline-containing phospholipids and/or glycosphingoplipids. Usually, the amphipathic molecules comprise one or more glycerophospholipids. As the skilled person will appreciate, glycerophospholipids include, but are not limited to glycerophospholipids having a structure as defined in the following formula (I):

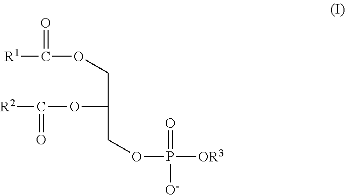

wherein:
R¹ and R², which are the same or different, are selected from C₁₀-C₂₅ alkyl groups and C₁₀-C₂₅ alkylene groups;
either R³ is absent such that OR³ is O⁻, or R³ is present and is H, CH₂CH₂N(R⁴)₃⁺, a sugar group, or an amino acid group; and
each R⁴, which is the same or different, is independently selected from H and unsubstituted C₁-C₄ alkyl.

Typically, when R³ is CH₂CH₂N(R⁴)₃⁺, each R⁴, which is the same or different, is selected from H and methyl. As the skilled person will appreciate, when each and every R⁴ is methyl, the R³ group is a choline group, and when each and every R⁴ is H, the R³ group is an ethanolamine group.

When R³ is an amino acid group it may for instance be a serine group, i.e. —CH₂CH(NH₂)(COOH). When R³ is a sugar group, it may for instance be glycerol, i.e. —CH₂CHOHCH₂OH, or for instance inositol, i.e. —CH(CHOH)₅.

Typical examples of R¹ and R² groups are C10-C25 alkyl groups, including, but not limited to linear C10-C25 alkyl groups such as, for instance, CH₃(CH₂)₁₀—, CH₃(CH₂)₁₂—, CH₃(CH₂)₁₄—, CH₃(CH₂)₁₆—, CH₃(CH₂)₁₈—, CH₃(CH₂)₂₂— and branched C10-C25 alkyl groups such as for instance —CH₂—CH(CH₃)—(CH₂)₃—CH(CH₃)—(CH₂)₃—CH(CH₃)—(CH₂)₃—CH(CH₃)₂.

Further typical examples of R¹ and R² groups are unsubstituted C₁₀-C₂₅ alkylene groups, including, but not limited to, CH₃(CH₂)₅CH═CH(CH₂)₇—, CH₃(CH₂)₇CH═CH(CH₂)₇—, CH₃(CH₂)₄CH═CHCH₂CH═CH(CH₂)₇—, CH₃(CH₂)₄(CH═CHCH₂)₃CH═CH(CH₂)₃—, and CH₃CH₂CH═CHCH₂CH═CHCH₂CH═CH(CH₂)₇—.

As the skilled person will appreciate, the O— group in the phosphate group adjacent to the OR³ group may in some embodiments be protonated, or associated with a suitable cation, for instance a metal cation such as Na⁺.

Thus, the amphipathic molecules may comprise one or more glycerophospholipids having the structure of formula (I) as defined above. For instance, the amphipathic molecules may comprise any one or more comprise one or more glycerophospholipids, typically one or more of the following glycerophospholipids: 1,2-diphytanoyl-sn-glycero-3-phosphocholine (DPhPC), 1,2-distearoyl-sn-glycero-3-phosphocholine (DSPC) 1,2-dipalmitoyl-sn-glycero-3-phosphocholine (DPPC), or 1,2-dipalmitoyl-sn-glycero-3-[phospho-rac-(1-glycerol)] (DPPG) can be employed as the amphiphilic molecules in the droplet, or a mixture of one or more thereof. The glycerophospholipid 1,2-dioleoyl-sn-glycero-3-phosphoethanolamine (DOPE) may also be used, and is typically used in combination with a pH-sensitive lipid, for instance a fatty acid. Preferably, the amphipathic molecules comprise 1,2-diphytanoyl-sn-glycero-3-phosphocholine (DPhPC).

Apparatus Features—Piezoelectric Transducer Voltage, Temperature Control, Tip,

The control unit of the apparatus is usually adapted to coordinate (a) the movement of the droplet receiving region relative to the or each droplet generator and (b) the dispensing of the droplets, to create said droplet assembly.

The control unit may, for instance, be adapted to control the movement of the container or the substrate. Additionally or alternatively, the control unit may be adapted to control the movement of the or each droplet generator. Typically, the control unit is adapted to control the movement of the container or substrate.

The apparatus of the invention usually further comprises a micromanipulator for moving the droplet receiving region relative to the or each droplet generator. Typically, the control unit is adapted to control movement of the droplet receiving region relative to the or each droplet generator using the micromanipulator.

More typically, the apparatus of the invention further comprises a micromanipulator for moving the droplet receiving region, and wherein the control unit is adapted to control movement of the container using the micromanipulator. The micromanipulator is generally a motorized micromanipulator. The droplet receiving region is typically disposed on the micromanipulator, so that the movement of the micromanipulator causes movement of the droplet receiving region. Typically, the control unit is adapted to control movement of the micromanipulator, which in turn causes movement of the droplet receiving region. The control unit is typically adapted to communicate with the micromanipulator for moving the container via an electrical or wireless signal. The control unit is typically in electrical connection with the micromanipulator. Alternatively, the control means is capable of communicating with the micromanipulator wirelessly.

The apparatus may further comprises a micromanipulator for moving the or each droplet generator and, when the apparatus comprises more than one said droplet generator, for coordinating the relative displacement of the droplet generators. Individual droplet generators may, for instance, be moved together or separately. The micromanipulator for moving the droplet receiving region may be the same as micromanipulator for moving the or each droplet generator. Alternatively, the micromanipulator for moving the droplet receiving region may be the different from micromanipulator for moving the or each droplet generator. Typically, the micromanipulator for moving the droplet receiving region is the same as micromanipulator for moving the or each droplet generator.

The control unit is typically adapted to communicate with the micromanipulator via an electrical or wireless signal. The control means is typically in electrical connection with the micromanipulator. Alternatively, the control means is capable of communicating with the micromanipulator wirelessly.

Usually, the droplet or each generator in the apparatus of the invention contains droplet medium. Usually, the control unit is adapted to control the dispensing of droplets of the aqueous medium from the or each droplet generator. As mentioned hereinbefore, the droplet medium is typically an aqueous medium as further defined herein, typically a natural extracellular matrix. Typically, the droplet medium comprises one or more biological cells as described herein.

Typically, the or each droplet generator comprises a chamber for holding a droplet medium (typically an aqueous medium, preferably a natural extracellular matrix material, e.g. matrigel); an outlet; and a component for displacing a volume of said droplet medium through said outlet and thereby dispensing said volume as a droplet. Typically, the or each droplet generator further comprises an inlet. Typically, the inlet allows entry of air into the chamber of the droplet generator upon the dispensing of a droplet from the outlet. The inlet may, for instance, be for introducing a droplet medium, typically an aqueous droplet medium, into the chamber. The chamber is typically filled with from 200 to 600 μl of the droplet medium, for instance from 400 to 500 μl. For instance, the chamber may be filled with about 400 μl of the droplet medium. Usually, the chamber is filled with the droplet medium through capillary action.

As the skilled person will appreciate, it may be possible for the droplet medium to evaporate from the chamber. Evaporation may have an impact on the diameter of droplets dispensed from the droplet generator. The evaporation may be prevented by having a layer of a hydrophobic medium on top of the aqueous droplet medium. Accordingly, the aqueous droplet medium may have a layer of a hydrophobic medium on top of it. The hydrophobic medium may be any suitable hydrophobic medium. Typically, the hydrophobic medium will be a hydrophobic medium as defined herein.

There may be some applications for which only a small quantity of an aqueous droplet medium is required. Thus, in other embodiments, the chamber is filled with from 0.5 to 50 μl of the aqueous medium, for instance from 1 to 10 μl. For instance, the chamber may be filled with about 5 μl. In these embodiments, the droplet generator is typically first filled with water. The outlet of the generator may then be immersed in a well comprising a hydrophobic medium, which hydrophobic medium may be as herein defined. Suction may then be applied at the inlet of the droplet generator, for instance, by using a micropipette. By doing this, the hydrophobic medium is drawn into the outlet. For instance, the amount of hydrophobic medium drawn into the outlet may be from 0.5 to 50 μl, for instance from 1 to 10 μl. The outlet may then be immersed into another well comprising the aqueous medium. Again, suction may be used to load from 0.5 to 50 μl of the aqueous medium, for instance from 1 to 10 μl, into the outlet. The hydrophobic medium forms a plug within the nozzle that prevents the aqueous medium in the outlet tip from mixing with the larger volume of water. Usually, the volume of water and the hydrophobic medium together transmit the pulse of pressure created by the piezoelectric transducer to the tip of the outlet, where a droplet is formed from the aqueous medium. The outlet may, for instance, comprise a nozzle.

Typically, the control unit comprises a computer or dedicated electronic hardware. More typically, the control unit comprises at least one computer. It usually comprises at least one personal computer (PC). The control unit may, for instance, be a PC. In some embodiments, the control unit comprises a PC and dedicated electronic hardware.

In the apparatus of the invention, the or each droplet generator may comprise: a chamber for holding droplet medium; an outlet; and a component for displacing a volume of said droplet medium through said outlet and thereby dispensing said volume as a droplet.

Typically, the at least one droplet generator is a piezoelectric droplet generator. More typically, the at least one droplet generator is a piezoelectric droplet generator which comprises a piezoelectric transducer for dispensing droplets. Thus, typically the component for displacing a volume of said droplet medium through said outlet is a piezoelectric transducer.

Typically, the apparatus of the invention comprises a piezoelectric driver which is capable of applying voltages more negative than −50 V and voltages more positive than +50 V to the piezoelectric transducer. For instance, the piezoelectric driver may be capable of applying voltages more negative than −100 V and voltages more positive than +100 V to the piezoelectric transducer. Preferably, the piezoelectric driver is capable of applying voltages more negative than −120 V and voltages more positive than +120 V to the piezoelectric transducer. For instance, the piezoelectric driver may be capable of applying voltages of −130 V and +130 V to the piezoelectric transducer.

Typically, the control unit is configured to control the application of a voltage pulse having a peak-to-peak amplitude of greater than 100 V to the piezoelectric transducer. Typically, the peak-to-peak amplitude is at least 120 V, preferably as least 150 V, and optionally at least 200 V. For instance, the control unit is configured to control the application of a voltage pulse having a peak-to-peak amplitude of greater than 250 V, for instance 260 V, to the piezoelectric transducer. Typically, each voltage pulse has a duration of from 10 to 1,500 μs, preferably from 50 to 1,000 μs, optionally from 100 to 800 μs.

Typically, the control unit is adapted to control the dispensing of droplets of the aqueous medium from the or each droplet generator, so that droplets are dispensed at a rate of from 0.01 to 100 s⁻¹, for instance, at a rate of from 0.01 to 50 s⁻¹. Usually, the control unit is adapted to control the dispensing of droplets of the aqueous medium from the or each droplet generator, so that droplets are dispensed at a rate of from 0.01 to 10 s⁻¹.

Usually, in the apparatus of the invention, the temperature controller comprises a thermoelectric module which is in contact with the droplet receiving region (e.g. the container), optionally wherein the thermoelectric module is a Peltier-based temperature control stage. The temperature controller may be for maintaining the temperature of the bulk medium. Typically, the temperature controller is for maintaining the temperature of the bulk medium during printing.

Typically, the temperature controller is capable of maintaining the temperature of the bulk medium at least at any temperature in the range of from −5° C. to +40° C. For instance, the temperature controller may be capable of maintaining the temperature of the bulk medium at least at any temperature in the range of from −15° C. to +80° C. Preferably, the temperature controller is capable of maintaining the temperature of the bulk medium at a temperature of less than 10° C. For instance, the temperature controller may be capable of maintaining the temperature of the bulk medium at a temperature of from 2° C. to 8° C., for instance at a temperature of about 5° C.

Typically, the outlet of the at least one droplet generator is submerged in the bulk medium. This advantageously ensures that the temperature of droplet medium present in the at least one droplet generator is maintained at the same temperature as the bulk medium. Thus, the temperature of the droplet medium being printed can be kept at a desired value by the temperature controller via controlling the temperature of the hydrophobic medium. Typically, the or each droplet generator further comprises a capillary connected to the chamber, wherein a tip of the capillary is said outlet, and the capillary is at least partially submerged in the bulk medium.

Typically, a surface of the outlet, preferably an inside surface of the outlet, has a hydrophilic surface treatment. The presence of a hydrophilic surface treatment is advantageous as it prevents hydrophobic bulk medium (e.g. oil) from entering the outlet. Preferably, the hydrophilic surface treatment provides said surface of the outlet with a positive charge. The hydrophilic surface treatment preferably comprises (3-aminopropyl)trimethyoxysilane.

Typically, the or each droplet generators further comprises a capillary connected to the chamber, wherein a tip of the capillary is said outlet. Usually, the or each droplet generator further comprises a capillary tube to the chamber, wherein the capillary is the nozzle and the tip of the capillary is said outlet. The tip of the capillary typically has a diameter of less than 150 μm. For instance the tip of the capillary may have a diameter of from 20 μm to 200 μm, for instance from 60 μm to 120 μm. The tip of the capillary may, for instance, have a diameter of about 100 μm.

Thus, typically an inside surface of the capillary and/or the tip of the capillary has a hydrophilic surface treatment, preferably wherein the hydrophilic surface treatment provides the inside surface and/or tip of the capillary with a positive charge, more preferably wherein the hydrophilic surface treatment comprises (3-aminopropyl)trimethyoxysilane.

Usually, the or each droplet generator is adapted to dispense droplets having a diameter of equal to or less than 1 mm, optionally equal to or less than 250 μm, equal to or less than 150 μm, equal to or less than 90 μm, or equal to or less than 40 μm, preferably wherein the or each droplet generator is adapted to dispense droplets having a diameter of from 10 μm to 250 μm. Typically, the or each droplet generator is adapted to dispense droplets having a volume of equal to or less than 2 μL. For instance, the or each droplet generator is adapted to dispense droplets having a volume of equal to or less than 100 nL, equal to or less than 350 pL, equal to or less than 150 pL, or equal to or less than 45 pL, for instance from 0.001 nL (1 pL) to 100 nL, or from 0.001 nL (1 pL) to 500 pL.

The apparatus and printing processes described herein allow a high degree of control over the droplet size. By controlling the concentration of the cells in the droplet medium, the number of cells in the droplets produced can be controlled. Thus, the present invention makes it possible to produce droplet assemblies wherein each droplet contains a small number of cells. This is particularly useful when the apparatus is used to produce a droplet array of the invention as defined herein. Typically, at least 20% of the droplets in the droplet assembly, preferably at least 30% of the droplets in the droplet assembly, more preferably at least 40% of the droplets in the droplet assembly, optionally at least 70% of the droplet assembly, for instance all of the droplets in the droplet assembly, each comprise a specific number, c, of biological cells. In some embodiments, particularly when the apparatus is used to produce a droplet array of the invention as defined herein, c may be a small number; for instance c may be an integer of from 1 to 20. For instance, c may be from 1 to 10, for instance c may be from 1 to 5, for example c may be 1.

Typically, the apparatus is adapted to produce the droplet assembly without using an extraneous scaffold material. Thus, the size of the droplet assembly and the pattern of droplets in the assembly are controlled exclusively by the number and position of the droplets. An extraneous scaffold material is typically a pre-formed solid component onto which the droplets are printed, which provides a rigid 3-dimensional structure. By printing droplet assemblies without an extraneous scaffold material, the present invention provides droplet assemblies which are a closer approximation to soft tissues in the body, and which therefore provide a more realistic approximation of such environments.

In some instances, the apparatus comprises a plurality of droplet generators. An advantage of the apparatus comprising a plurality of said droplet generators is that each droplet generator may comprise a different droplet medium (typically aqueous medium). This allows diverse droplet assemblies to be printed. The droplet assemblies may, for instance, comprise multiple compartments with different types of biological cells thereby providing a means to study how the different cell types interact depending on the design of the droplet assembly.

Accordingly, when the apparatus comprises a plurality of said droplet generators, the apparatus usually comprises a first droplet generator comprising a first droplet medium and a second droplet generator comprising a second droplet medium, wherein the first and second droplet media are different. The first droplet medium is usually an aqueous medium as herein defined. The second droplet medium is typically also an aqueous medium as herein defined. Accordingly, in some embodiments, when the apparatus comprises a plurality of said droplet generators, the apparatus comprises a first droplet generator comprising a first aqueous medium and a second droplet generator comprising a second aqueous medium, wherein the first and second aqueous media are different. The first aqueous medium is usually an aqueous medium as herein defined. The second aqueous medium is typically an aqueous medium as herein defined.

Typically, the first droplet medium comprises biological cells of a first type and either the second droplet medium comprises biological cells of a second type which is different from the first type or the second droplet medium does not comprise biological cells. The biological cells in the first and second droplet media may be any biological cells as described herein.

Typically the biological cells of the first type and the biological cells of the second type are different types of cells found in soft tissue. Typically, the biological cells of the first type and the biological cells of the second type are different types of brain cells, typically different types of human brain cells, optionally wherein the biological cells of the first type comprise one of neural stem cells, neural progenitor cells, neurons, astrocytes, microglia and endothelial cells, and the biological cells of the second type, where present, comprise a different one of neural stem cells, neurons, astrocytes, microglia and endothelial cells, preferably wherein the biological cells of the first type comprise neural stem cells or cortical neurons and optionally wherein the biological cells of the second type, where present, comprise astrocytes.

Features of Droplet Assembly

The droplet assembly comprises a plurality of droplets, wherein each of said droplets comprises a droplet medium. Typically, the number of droplets in said plurality of droplets is greater than 10, preferably greater than 100, optionally greater than 1000, for example greater than 10000. For instance, the number of droplets in said plurality of droplets may be greater than 20000, greater than 50000 or greater than 100000.

The droplet assembly may be an extended two or three-dimensional network of said droplets. For instance an extended two or three-dimensional network of said plurality of droplets wherein the number of droplets in said plurality is greater than 10, preferably greater than 100, optionally greater than 1000, for example greater than 10000. For instance, the number of droplets in said plurality of droplets may be greater than 20000, greater than 50000 or greater than 100000.

For instance, the apparatus may be adapted to produce a droplet assembly wherein at least one of said droplets contacts another of said droplets to form a layer of said amphipathic molecules as an interface between the contacting droplets. Typically, the apparatus is adapted to produce a droplet assembly wherein each of said droplets contacts another of said droplets to form a layer (for instance a bilayer) of said amphipathic molecules as an interface between the contacting droplets. The number of droplets in said plurality may be greater than 10, and is preferably greater than 100, optionally greater than 1000, for example greater than 10000. For instance, the number of droplets in said plurality of droplets may be greater than 20000, greater than 50000 or greater than 100000.

For instance, the control unit may be adapted to coordinate (a) the movement of the droplet receiving region relative to the or each droplet generator and (b) the dispensing of the droplets, to create a said droplet assembly which comprises at least one layer of droplets, wherein each of said droplets comprises (i) said droplet medium and (ii) an outer layer of amphipathic molecules around the surface of the droplet medium, and wherein each droplet in the layer contacts at least one other droplet in the layer to form a layer of said amphipathic molecules as an interface between contacting droplets. Typically, the control unit is adapted to coordinate (a) the movement of the droplet receiving region relative to the or each droplet generator and (b) the dispensing of the droplets, to create a said droplet assembly which comprises a plurality of said layers of said droplets, wherein each layer is disposed adjacent to another layer, so that droplets in a layer contact droplets in an adjacent layer to form layers of amphipathic molecules as interfaces between the contacting droplets. Thus, the apparatus may be adapted to produce a droplet assembly wherein each of said droplets contacts another of said droplets to form multiple stacked layers, wherein each droplet in each layer contacts at least one other droplet in the same layer to form a layer of said amphipathic molecules as an interface between contacting droplets and wherein droplets in a layer contact droplets in an adjacent layer to form layers of amphipathic molecules as interfaces between the contacting droplets in different layers. The number of droplets may be greater than 10, and is preferably greater than 100, optionally greater than 1000, for example greater than 10000. For instance, the number of droplets may be greater than 20000, greater than 50000 or greater than 100000.

Typically, the control unit is adapted to coordinate (a) the movement of the droplet receiving region relative to the or each droplet generator and (b) the dispensing of the droplets, to create a said droplet assembly in which the droplets are arranged in a predetermined shape or in an ordered pattern.

For instance, the control unit may be adapted to coordinate (a) the movement of the droplet receiving region relative to the or each droplet generator and (b) the dispensing of the droplets, to create a said droplet assembly which comprises a plurality of droplets, wherein each of said droplets comprises (i) said droplet medium, and (ii) said outer layer of amphipathic molecules around the surface of the droplet medium, and wherein each of said droplets contacts another of said droplets to form a layer of said amphipathic molecules as an interface between the contacting droplets, wherein the plurality of droplets comprises a first region of said droplets and a second region of said droplets, wherein each droplet in the first region contacts at least one other droplet in the first region to form a layer of said amphipathic molecules as an interface between the contacting droplets, and each droplet in the second region contacts at least one other droplet in the second region to form a layer of said amphipathic molecules as an interface between the contacting droplets, wherein the droplet medium of the droplets in the first region is a first droplet medium and the droplet medium of the droplets in the first region is a second droplet medium wherein the first droplet medium and the second droplet medium are different. The first droplet medium and the second droplet medium may be any droplet media as described herein. The droplet assembly may comprise multiple regions comprising droplets of different droplet media in each.

Typically, the first droplet medium comprises biological cells of a first type and the second droplet medium comprises biological cells of a second type which is different from the first type. Alternatively, the first droplet medium may comprises biological cells of a first type and the second droplet medium may not comprise biological cells. The droplet assembly may comprise multiple regions, wherein the droplet medium of the droplets in each region differs from that in the other regions.

The biological cells of the first type and the biological cells of the second type may be any type of biological cells as described herein. Typically, the biological cells of the first type and the biological cells of the second type are different types of mammalian cells, preferably different types of human cells. For instance, the biological cells of the first type and the biological cells of the second type may be different types of any type of human cell, for instance different types of liver cells, ovarian cells, breast cells or brain cells. The biological cells may be cancer cells. Typically the biological cells are selected from cell types found in soft tissue. Typically, the biological cells of the first type and the biological cells of the second type are different types of brain cells, typically human brain cells, optionally wherein the biological cells of the first type comprise one of neural stem cells, neural progenitor cells, neurons, astrocytes, microglia and endothelial cells, and the biological cells of the second type, where present, comprise a different one of neural stem cells, neural progenitor cells, neurons, astrocytes, microglia and endothelial cells. For instance, the biological cells of the first type may comprise neural stem cells or cortical neurons and the biological cells of the second type, where present, may comprise astrocytes. The first and second types of biological cells may be cells from different corticol layers, typically different corticol layers in the human brain. For instance, the biological cells of the first type may be biological cells from one corticol layer and the biological cells of the second type may be biological cells from another, different coritcol layer. Typically, the first type of biological cell may comprise neurons with or without astrocytes from specific cortical layer and the second type may comprise neurons with/without astrocytes from another specific cortical layer. There are six cortical layers in human brain that consist of different types of neurons. 3D printing may be used pattern these layers together to provide layered cortical tissue as mentioned in Example 1.

The droplets of the first region and the droplets of the second region may be arranged in an ordered pattern. Thus the droplets of the first region and the droplets of the second region may be arranged in a two dimensional pattern or a three-dimensional pattern, preferably in a three dimensional pattern. A three-dimensional pattern typically comprises a network of droplets that is more than one droplet across in each of the three dimensions, for instance which comprises multiple stacked layers of droplets. The term “ordered pattern”, as used herein, refers to an arrangement in which the droplets are not simply positioned at random. For instance, the droplets of the first region and the droplets of the second region may be arranged such that the first region is adjacent the second region, or wherein the first region is an internal region and the second region is an external region which encases the internal region.

Typically, the or each layer of said amphipathic molecules which is an interface between contacting droplets is a bilayer of said amphipathic molecules. The bilayer comprises amphipathic molecules from the outer layer of amphipathic molecules around the surface of the aqueous medium of each droplet at the interface. The bilayer forms as it is an energetically more favourable configuration for the amphipathic molecules to adopt. The contacting droplets will acquire the geometry with the lowest free surface energy.

Alternatively, the droplet assembly may comprise a plurality of elements spaced apart from one another on a substrate in a bulk medium, wherein each element comprises at least one droplet which comprises droplet medium.

The apparatus may be adapted to produce said droplet assembly wherein the droplets are arranged in a droplet array as described herein. Typically a droplet array comprises a plurality of elements spaced apart from one another, wherein each element comprises at least one droplet which comprises a droplet medium which comprises one or more biological cells and a natural extracellular matrix material (for instance matrigel).

For instance, the apparatus may be adapted to produce said droplet assembly wherein the droplets are arranged in an array. Optionally, in said droplet assembly, individual droplets which are not in contact with one another are arranged in an array. Alternatively, optionally, in said droplet assembly, pairs of contacting droplets (which pairs are not in contact with one another—i.e. the pairs are spaced apart from each other) are arranged in an array. Optionally, in said droplet assembly, groups of at least three (optionally contacting) droplets, which groups are not in contact with one another, are arranged in an array.

Process for Producing Droplet Assembly Using Apparatus

The invention also provides a process for producing a droplet assembly using an apparatus for producing the droplet assembly, which droplet assembly comprises: a plurality of droplets, wherein each of said droplets comprises a droplet medium; which apparatus comprises: at least one droplet generator wherein the droplet generator is suitable for generating droplets of a viscous droplet medium; a droplet receiving region which is moveable relative to the at least one droplet generator; a temperature controller; and a control unit, which control unit is adapted to control the dispensing of droplets from the at least one droplet generator and the movement of the droplet receiving region relative to the at least one droplet generator; wherein said droplet receiving region further comprises a bulk medium, wherein the bulk medium and the droplet medium are immiscible; which process comprises:

(a) a plurality of dispensing steps, wherein each dispensing step comprises dispensing a droplet of the droplet medium from a said droplet generator into the bulk medium, and thereby forming in the bulk medium a droplet which comprises said droplet medium; and

(b) moving the droplet receiving region relative to the at least one droplet generator, to control the relative positioning of the droplets in the bulk medium.

The droplet assembly may be any droplet assembly as described herein. The apparatus may be any apparatus as described herein. The droplet medium may be any droplet medium as described herein. Typically, the droplet medium is aqueous. Preferably, the droplet medium comprises an undiluted natural extracellular matrix material and biological cells. More preferably the droplet medium comprises, or consists of, undiluted matrigel and biological cells. The biological cells may be any biological cells as described herein. The bulk medium may be any bulk medium as described herein. The droplet generator may be any droplet generator as described herein. The temperature controller may be any temperature controller as described herein. The control unit may be any control unit as described herein. The droplet receiving region may be any droplet receiving region as described herein.

Preferably, the process is a process for producing a droplet assembly comprising: a plurality of droplets, wherein each of said droplets comprises: (i) a droplet medium, and (ii) an outer layer of amphipathic molecules around the surface of the droplet medium, the process comprising:

(a) a plurality of dispensing steps, wherein each dispensing step comprises dispensing a droplet of the droplet medium from a said droplet generator into the bulk medium, in the presence of amphipathic molecules, and thereby forming in the bulk medium a droplet which comprises (i) said droplet medium and (ii) an outer layer of amphipathic molecules around the surface of the droplet medium; and

(b) moving the container relative to the at least one droplet generator, to control the relative positioning of the droplets in the bulk medium.

On the other hand, the droplet assembly may not comprise any amphipathic molecules. In this case, no amphipathic molecules are present in the process.

Typically, the number of droplets in said plurality of droplets, and the number of dispensing steps, is greater than 10, preferably greater than 100, optionally greater than 1000, for example greater than 10000. For instance, the number of droplets in said plurality of droplets, and the number of dispensing steps, may be greater than 20000, greater than 50000 or greater than 100000.

The droplet assembly comprises a plurality of droplets, wherein each of said droplets comprises a droplet medium. The droplet assembly may be an extended two or three-dimensional network of droplets. The number of droplets may be greater than 10, and is preferably greater than 100, optionally greater than 1000, for example greater than 10000. For instance, the number of droplets may be greater than 20000, greater than 50000 or greater than 100000.

Alternatively, the droplet assembly may comprise a plurality of elements spaced apart from one another on a substrate in a bulk medium, wherein each element comprises at least one droplet which comprises droplet medium.

Preferably, the droplet assembly comprises a plurality of droplets, wherein each of said droplets comprises a droplet medium. The droplet assembly may be an extended two or three-dimensional network of droplets. The number of droplets may be greater than 10, and is preferably greater than 100, optionally greater than 1000, for example greater than 10000. For instance, the number of droplets may be greater than 20000, greater than 50000 or greater than 100000. For instance, the process may produce a droplet assembly wherein at least one of said droplets contacts another of said droplets to form a layer of said amphipathic molecules as an interface between the contacting droplets. Preferably, each of said droplets contacts another of said droplets to form a layer of said amphipathic molecules (for instance a bilayer of said amphipathic molecules) as an interface between the contacting droplets.

For instance, the control unit may coordinate (a) the movement of the droplet receiving region relative to the or each droplet generator and (b) the dispensing of the droplets, to create a said droplet assembly which comprises at least one layer of droplets, wherein each of said droplets comprises (i) said droplet medium and (ii) an outer layer of amphipathic molecules around the surface of the droplet medium, and wherein each droplet in the layer contacts at least one other droplet in the layer to form a layer of said amphipathic molecules as an interface between contacting droplets. Typically, the control unit coordinates (a) the movement of the droplet receiving region relative to the or each droplet generator and (b) the dispensing of the droplets, to create a said droplet assembly which comprises a plurality of said layers of said droplets, wherein each layer is disposed adjacent to another layer, so that droplets in a layer contact droplets in an adjacent layer to form layers (e.g. bilayers) of amphipathic molecules as interfaces between the contacting droplets. Thus, the process may produce a droplet assembly wherein each of said droplets contacts another of said droplets to form multiple stacked layers, wherein each droplet in each layer contacts at least one other droplet in the same layer to form a layer (e.g. bilayer) of said amphipathic molecules as an interface between contacting droplets and wherein droplets in a layer contact droplets in an adjacent layer to form layers (e.g. bilayers) of amphipathic molecules as interfaces between the contacting droplets in different layers. The number of droplets is preferably greater than 100, optionally greater than 1000, for example greater than 10000. For instance, the number of droplets may be greater than 20000, greater than 50000 or greater than 100000.

Typically, the process produces a droplet assembly that comprises: said plurality of droplets, wherein each of said droplets comprises: (i) a droplet medium, and (ii) an outer layer of amphipathic molecules around the surface of the droplet medium, wherein at least one of said droplets contacts another of said droplets to form a layer of said amphipathic molecules as an interface between the contacting droplets, and preferably each of said droplets contacts another of said droplets to form a layer of said amphipathic molecules as an interface between the contacting droplets. Preferably wherein the or each layer of said amphipathic molecules which is an interface between contacting droplets is a bilayer of said amphipathic molecules.

Moving the droplet receiving region relative to the at least one droplet generator may comprise: moving the droplet receiving region relative to the at least one droplet generator to position at least one of said droplets adjacent to another of said droplets so that at least one of said droplets contacts another of said droplets to form a layer of said amphipathic molecules as an interface between the contacting droplets. Preferably, moving the droplet receiving region relative to the at least one droplet generator comprises: moving the droplet receiving region relative to the at least one droplet generator to position each droplet adjacent to at least one other droplet, so that each of said droplets contacts another of said droplets to form a layer of said amphipathic molecules as an interface between contacting droplets. Typically, the or each layer of said amphipathic molecules which is an interface between contacting droplets is a bilayer of said amphipathic molecules. The number of droplets may be greater than 10, and is preferably greater than 100, optionally greater than 1000, for example greater than 10000. For instance, the number of droplets may be greater than 20000, greater than 50000 or greater than 100000.

Typically, the droplets are arranged in a predetermined shape or in an ordered pattern.

Typically, each of said droplets has a diameter of equal to or less than 1 mm, optionally equal to or less than 250 μm, equal to or less than 150 μm, equal to or less than 90 μm, or equal to or less than 40 μm, preferably wherein each of said droplets has a diameter of from 10 μm to 250 μm.

Typically, each of said droplets has a volume of equal to or less than 2 μL, preferably wherein each of said droplets has a volume of equal to or less than 100 nL, equal to or less than 350 pL, equal to or less than 150 pL, or equal to or less than 45 pL, for instance from 0.001 nL (1 pL) to 100 nL, or from 0.001 nL (1 pL) to 500 pL.

The apparatus and printing processes described herein allow a high degree of control over the droplet size. By controlling the concentration of the cells in the droplet medium, the number of cells in the droplets produced can be controlled. Thus, the present invention makes it possible to produce droplet assemblies wherein each droplet contains a small number of cells, which is especially applicable when a droplet array as defined herein is being produced. Typically, at least 20% of the droplets in the droplet assembly, preferably at least 30% of the droplets in the droplet assembly, more preferably at least 40% of the droplets in the array, optionally at least 70% of the droplet assembly, for instance all of the droplets in the droplet assembly, each comprise a specific number, c, of biological cells. Typically, and especially when a droplet array as defined herein is being produced, c is an integer of from 1 to 20. For instance, c may be from 1 to 10, or c may be from 1 to 5, for instance c may be 1.

The droplet assembly may comprise a plurality of droplets, wherein each of said droplets comprises a droplet medium, wherein the droplets are arranged in an array. Each of said droplets may comprise (i) a droplet medium, and (ii) an outer layer of amphipathic molecules around the surface of the droplet medium. Typically, the droplet assembly comprises a plurality of droplets, wherein individual droplets which are not in contact with one another are arranged in an array. Typically, the outer layer of amphipathic molecules, when present, on each droplet in the array is not in contact with the outer layer of amphipathic molecules on any other droplet in the array. In other words, the droplets in the array are spaced apart so that no layer/bilayer forms between droplets. The droplet assembly may comprise a plurality of droplets wherein pairs of contacting droplets which pairs are not in contact with one another are arranged in an array. The droplet assembly may comprise a plurality of droplets wherein groups of at least three (optionally contacting) droplets, which groups are not in contact with one another, are arranged in an array. Typically, moving the droplet receiving region relative to the at least one droplet generator, to control the relative positioning of the droplets in the bulk medium, comprises moving the droplet receiving region relative to the at least one droplet generator to position each droplet to arrange the droplets in said array.

The droplet medium may be any droplet medium as described herein. Typically, the droplet medium is aqueous. Preferably, the droplet medium comprises an undiluted natural extracellular matrix material and biological cells. More preferably the droplet medium comprises, or consists of, undiluted matrigel and biological cells. The biological cells may be any biological cells as described herein.

The bulk medium may be any bulk medium as described herein. Typically, the bulk medium is a hydrophobic medium, for instance an oil or mixture of oils. Preferably, the bulk medium comprises silicone oil and a hydrocarbon, optionally wherein the hydrocarbon is a straight-chained, unsubstituted C₈₋₁₅ alkane, and preferably wherein the hydrocarbon is undecane. Typically, the ratio by volume of the hydrocarbon and the silicone oil is selected such that the density of the hydrophobic medium matches that of the droplet medium at the printing temperature.

The bulk medium may comprise or consist of a naturally occurring oil, for instance sunflower oil. The bulk medium may comprise or consist of the primary constituent of naturally occurring oil, for instance, the bulk medium may comprise or consist of glyceryl trioleate and/or glyceryl trilinoleate.

When amphipathic molecules are used, the bulk medium, or the droplet medium, or both, may further comprise the amphipathic molecules. The amphipathic molecules may be any amphipathic molecules as described herein. For instance, the amphipathic molecules may comprise one or more glycerophospholipids, preferably DPhPC.

Typically, the at least one droplet generator is a piezoelectric droplet generator which comprises a piezoelectric transducer for dispensing droplets, and wherein each dispensing step comprises applying a voltage pulse to the piezoelectric transducer. The piezoelectric transducer may be any piezoelectric transducer as described herein. The voltage pulse may be any voltage pulse as described herein. Typically, applying the voltage pulse comprises applying a voltage more negative than −50 V or a voltage more positive than +50 V to the piezoelectric transducer, preferably applying the voltage pulse comprises applying a voltage more negative than −100 V or a voltage more positive than +100 V to the piezoelectric transducer, more preferably applying the voltage pulse comprises applying a voltage more negative than −120 V or a voltage more positive than +120 V to the piezoelectric transducer. For instance, applying the voltage pulse may comprise applying a voltage of −130 V or a voltage of +130 V to the piezoelectric transducer. The voltage pulse may have a peak-to-peak amplitude of greater than 100 V to the piezoelectric transducer, preferably wherein said peak-to-peak amplitude is at least 120 V, more preferably at least 150 V, and optionally at least 200 V or at least 250 V, for instance about 260 V. Typically, the voltage pulse has a duration of from 10 to 1,500 μs, preferably from 50 to 1,000 μs, optionally from 100 to 800 μs.

Typically, in the process of the invention the droplets are dispensed at a rate of at least 0.01 s⁻¹, preferably at a rate of at least 0.25 s⁻¹, more preferably at a rate of at least 0.5 s⁻¹, optionally at a rate of at least 1 s⁻¹, for instance at a rate of at least 2 s⁻¹, at a rate of at least 5 s⁻¹, or at a rate of at least 10 s⁻¹.

Typically, in the process of the invention during at least one dispensing step, and preferably during each dispensing step, the temperature controller maintains the temperature of the bulk medium at a droplet printing temperature. Usually, during at least one dispensing step, and preferably during each dispensing step, the outlet of the at least one droplet generator is submerged in the bulk medium and the temperature controller maintains the temperature of the bulk medium at a droplet printing temperature.

Typically, the or each droplet generator further comprises a capillary connected to the chamber, wherein a tip of the capillary is the outlet, and during at least one dispensing step, and preferably during each dispensing step, the capillary is at least partially submerged in the bulk medium and the temperature controller maintains the temperature of the bulk medium at a droplet printing temperature.

The droplet medium may be capable of gelation in response to a stimulus. Typically, gelation is induced by a change in temperature, for instance heating or cooling the droplet medium from the temperature at the time of generating a droplet. Preferably, the droplet medium is capable of gelation on heating. Thus, the droplet printing temperature is typically a temperature below a temperature at which the droplet medium is capable of forming a gel. Preferably, the droplet printing temperature is a temperature of less than 10° C., preferably a temperature of from 2° C. to 8° C., for instance at a temperature of about 5° C.

In the process of the invention, the plurality of dispensing steps may comprise: a first set of dispensing steps, which together produce a first region of said droplets in the bulk medium, wherein each droplet in the first region contacts at least one other droplet in the first region to form a layer of said amphipathic molecules as an interface between contacting droplets; and a second set of dispensing steps, which together produce a second region of said droplets in the bulk medium, wherein each droplet in the second region contacts at least one other droplet in the second region to form a layer of said amphipathic molecules as an interface between contacting droplets, wherein the droplet medium of the droplets in the first region is a first droplet medium and the droplet medium of the droplets in the second region is a second droplet medium wherein the first droplet medium and the second droplet medium are different. The first droplet medium and the second droplet medium may be any droplet media as described herein. Typically, the first droplet medium comprises biological cells of a first type and the second droplet medium comprises biological cells of a second type which is different from the first type. Alternatively, the first droplet medium may comprise biological cells of a first type and the second droplet medium may not comprise biological cells.

The biological cells of the first type and the biological cells of the second type may be any type of biological cells as described herein. Typically the biological cells are selected from cell types found in soft tissue. Typically, the biological cells of the first type and the biological cells of the second type are different types of brain cells, typically human brain cells, optionally wherein the biological cells of the first type comprise one of neural stem cells, neural progenitor cells, neurons, astrocytes, microglia and endothelial cells, and the biological cells of the second type, where present, comprise a different one of neural stem cells, neural progenitor cells, neurons, astrocytes, microglia and endothelial cells. Preferably wherein the biological cells of the first type comprise neural stem cells or cortical neurons and optionally wherein the biological cells of the second type, where present, comprise astrocytes. The first and second types of biological cells may be cells from different corticol layers, typically different corticol layers in the human brain. For instance, the biological cells of the first type may be biological cells from one corticol layer and the biological cells of the second type may be biological cells from another, different coritcol layer. Typically, the first type of biological cell may comprise neurons with or without astrocytes from specific cortical layer and the second type may comprise neurons with/without astrocytes from another specific cortical layer. There are six cortical layers in human brain that consist of different types of neurons. 3D printing may be used pattern these layers together to provide layered cortical tissue as mentioned in Example 1.

Typically, the droplets of the first region and the droplets of the second region are arranged in an ordered pattern. Thus the droplets of the first region and the droplets of the second region may be arranged in a two dimensional pattern or a three-dimensional pattern, preferably in a three dimensional pattern. A three-dimensional pattern typically comprises a network of droplets that is more than one droplet across in each of the three dimensions, for instance which comprises multiple stacked layers of droplets. The term “ordered pattern”, as used herein, refers to an arrangement in which the droplets are not simply positioned at random. For instance, the droplets of the first region and the droplets of the second region may be arranged such that the first region is adjacent the second region, or wherein the first region is an internal region and the second region is an external region which encases the internal region.

Typically, in the process of the invention, after the plurality of dispensing steps and the production of said droplet assembly in the bulk medium, the temperature of the bulk medium is increased by the temperature controller to a temperature which causes gelation of the droplet medium. For instance, after the plurality of dispensing steps and the production of said droplet assembly in the bulk medium, the temperature of the bulk medium may be increased by the temperature controller to a temperature of from 10° C. to 30° C., preferably to a temperature of from 20° C. to 30° C., for instance about 25° C. Typically, the temperature of the bulk medium is held at said temperature for at least 15 minutes.

The temperature of the bulk medium may be further increased by the temperature controller, for instance to a temperature of from 30° C. to 40° C., preferably to a temperature of about 37° C.

The process typically comprises recovering said droplet assembly from the bulk medium.

Recovering said droplet assembly from the bulk medium may comprise removing said droplet assembly from the bulk medium. Typically, however, recovering said droplet assembly from the bulk medium comprises transferring said droplet assembly to another medium.

Typically, recovering said droplet assembly from the bulk medium comprises transferring said droplet assembly into an aqueous medium, preferably wherein the aqueous medium is cell culture medium. Recovering said droplet assembly from the bulk medium may comprise replacing at least part of the bulk medium with fresh bulk medium in order to reduce the concentration of amphipathic molecules in the bulk medium, and then replacing the bulk medium with an aqueous medium, preferably wherein the aqueous medium is cell culture medium.

The invention also provides a droplet assembly which is obtainable by a process as described herein.

Process for Producing a Droplet Assembly of Amphipathic-Connected Droplets of Natural ECM And Cells

The invention also provides a process for producing a droplet assembly, the process comprising
generating, in a bulk medium, a plurality of droplets, wherein each of said droplets comprises: (i) a droplet medium which comprises biological cells and a natural extracellular matrix material, and (ii) an outer layer of amphipathic molecules around the surface of the droplet medium, wherein the bulk medium and the droplet medium are immiscible, and
contacting each of said droplets with another of said droplets to form a layer of said amphipathic molecules as an interface between contacting droplets.

The bulk medium may be any bulk medium as described herein. The amphipathic molecules may be any amphipathic molecules as described herein.

Typically, the number of droplets in said plurality is greater than 10, preferably greater than 100, optionally greater than 1000, for example greater than 10000. For instance, the number of droplets in said plurality of droplets may be greater than 20000, greater than 50000 or greater than 100000. The droplet assembly is typically an extended two or three-dimensional network of droplets, preferably an extended three-dimensional network of droplets. The droplet assembly may comprise at least one layer of droplets, wherein each of said droplets comprises (i) said droplet medium and (ii) an outer layer of amphipathic molecules around the surface of the droplet medium, and wherein each droplet in the layer contacts at least one other droplet in the layer to form a layer of said amphipathic molecules as an interface between contacting droplets. The droplet assembly may preferably comprise a plurality of layers of said droplets, wherein each layer is disposed adjacent to another layer, so that droplets in a layer contact droplets in an adjacent layer to form layers of amphipathic molecules as interfaces between the contacting droplets. For instance the layers of droplets may be stacked on top of each other to form a three-dimensional structure. The number of droplets in said structure is typically greater than 100, for instance greater than 1000, and preferably greater than 10000. For instance, the number of droplets may be greater than 20000, greater than 50000 or greater than 100000.

Typically, each layer of said amphipathic molecules which is an interface between contacting droplets is a bilayer of said amphipathic molecules. The amphipathic molecules typically comprise one or more glycerophospholipids, optionally wherein the amphipathic molecules comprise 1,2-diphytanoyl-sn-glycero-3-phosphocholine (DPhPC).

The droplet medium may be any droplet medium comprising biological cells, as described herein, and a natural extracellular matrix material, as described herein. The natural extracellular matrix material in the droplet medium is typically in liquid form and is capable of gelation on heating.

Typically, the droplet medium comprises biological cells at a density of at least 10⁴ cells/mL typically at least 10⁵ cells/mL preferably at least 10⁶ cells/mL. Typically, the droplet medium comprises biological cells at a density of up to 10⁸ cells/mL, for instance of up to 7.5×10⁷ cells/mL or up to 5×10⁷ cells/mL. For instance, the droplet medium may comprise biological cells at a density of from 1×10⁶ cells/mL to 7.5×10⁷ cells/mL, or from 1×10⁶ cells/mL to 5×10⁷ cells/mL, or from 1×10⁶ cells/mL to 4×10⁷ cells/mL. The cells may be any biological cells, for instance cells originating from any plant, animal, fungus or single-celled organism. Typically, the cells are mammalian cells, preferably human cells. For instance, the biological cells may be human liver cells, ovarian cells, breast cells or brain cells. The biological cells may be cancer cells. Typically the biological cells are selected from cell types found in soft tissue, for instance brain cells. Thus, the biological cells may comprise brain cells, typically human brain cells. The brain cells may comprise neural stem cells, neural progenitor cells, neurons, astrocytes, microglia or endothelial cells. Preferably, the brain cells comprise neural stem cells.

The natural extracellular matrix material is preferably matrigel. For instance, the droplet medium may comprise matrigel and the biological cells. It preferably comprises matrigel and mammalian cells, for instance matrigel and brain cells, typically matrigel and human brain cells. Typically, the natural extracellular matrix material is undiluted. The droplet medium may therefore comprise an undiluted natural extracellular matrix material and biological cells. Preferably the droplet medium comprises undiluted matrigel and biological cells.

Typically, the natural extracellular matrix material and the biological cells together form at least 80% by volume of the droplet medium, preferably at least 90% by volume of the droplet medium, more preferably at least 95% by volume of the droplet medium, for instance at least 99% by volume of the droplet medium. Typically, the droplet medium consists of the natural extracellular matrix material and the biological cells, preferably wherein the natural extracellular matrix material is matrigel.

Preferably the droplet medium comprises undiluted natural extracellular matrix material and biological cells, more preferably wherein the droplet medium comprises undiluted matrigel and biological cells. For instance, the droplet medium may consist of matrigel and biological cells. The droplet medium may comprise collagen and biological cells.

The droplet assembly is usually produced without using an extraneous scaffold material.

In the process of the invention, the steps of generating the droplets in the bulk medium and contacting each of the droplets with another of the droplets is typically performed at a droplet printing temperature. Typically, the steps of generating the droplets in the bulk medium and contacting each of the droplets with another of the droplets are performed while the temperature of the bulk medium is maintained at a droplet printing temperature. The droplet printing temperature may be any droplet printing temperature as described herein. Typically, the droplet printing temperature is a temperature below a temperature at which the droplet medium is capable of forming a gel. Thus, the droplet printing temperature may be a temperature of less than 10° C., preferably a temperature of from 2° C. to 8° C., for instance at a temperature of about 5° C.

Typically, the steps of generating the droplets in the bulk medium and contacting each of the droplets with another of the droplets comprise arranging the droplets in a predetermined shape or in an ordered pattern. Thus the droplets of the first region and the droplets of the second region may be arranged in a two dimensional pattern or a three-dimensional pattern, preferably in a three dimensional pattern. A three-dimensional pattern typically comprises a network of droplets that is more than one droplet across in each of the three dimensions, for instance which comprises multiple stacked layers of droplets. The term “ordered pattern”, as used herein, refers to an arrangement in which the droplets are not simply positioned at random. For instance, the droplets of the first region and the droplets of the second region may be arranged such that the first region is adjacent the second region, or wherein the first region is an internal region and the second region is an external region which encases the internal region.

For instance, the process may comprise generating, in said bulk medium, a plurality of first droplets, wherein each of said first droplets comprises: (i) a first droplet medium which comprises biological cells of a first type and a natural extracellular matrix material, and (ii) an outer layer of amphipathic molecules around the surface of the first droplet medium, and contacting each of said first droplets with another of said first droplets to form a layer of said amphipathic molecules as an interface between contacting droplets; and generating, in said bulk medium, a plurality of second droplets, wherein each of said second droplets comprises: (i) a second droplet medium which comprises a natural extracellular matrix material, and (ii) an outer layer of amphipathic molecules around the surface of the second droplet medium, and contacting each of said second droplets with another of said second droplets to form a layer of said amphipathic molecules as an interface between contacting droplets; wherein at least one of the first droplets contacts at least one of the second droplets to form a layer of said amphipathic molecules as an interface between contacting droplets; wherein the first droplet medium and the second droplet medium are different.

The biological cells of the first type may be any type of biological cells as described herein. Typically the biological cells are selected from cell types found in soft tissue. For instance, the biological cells of the first type are brain cells, typically human brain cells, optionally wherein the biological cells of the first type comprise brain cells which are neural stem cells, neural progenitor cells, neurons, astrocytes, microglia or endothelial cells. Preferably the biological cells of the first type comprise neural stem cells or cortical neurons.

The second droplet medium may comprise biological cells of a second type which is different from the first type. Alternatively, the second droplet medium may not comprise biological cells. The biological cells of the second type may be any type of biological cells as described herein. Typically the biological cells are selected from cell types found in soft tissue. The biological cells of the second type may be a second type of brain cell, typically a human brain cell, optionally wherein the biological cells of the second type comprise brain cells which are neural stem cells, neural progenitor cells, neurons, astrocytes, microglia or endothelial cells, optionally wherein the biological cells of the second type comprise astrocytes.

The first and second types of biological cells may be cells from different corticol layers, typically different corticol layers in the human brain. For instance, the biological cells of the first type may be biological cells from one corticol layer and the biological cells of the second type may be biological cells from another, different coritcol layer. Typically, the first type of biological cell may comprise neurons with or without astrocytes from specific cortical layer and the second type may comprise neurons with/without astrocytes from another specific cortical layer. There are six cortical layers in human brain that consist of different types of neurons. 3D printing may be used pattern these layers together to provide layered cortical tissue as mentioned in Example 1.

Thus, typically, the steps of generating the droplets in the bulk medium and contacting each of the droplets with another of the droplets comprise: arranging the plurality of first droplets and the plurality of second droplets in an ordered pattern, optionally wherein the plurality of first droplets is adjacent to the plurality of second droplets, or wherein the plurality of first droplets forms an internal region and the plurality of second droplets forms an external region which encases the internal region.

The droplet assembly may be produced by this process using an apparatus of the invention as defined herein.

The invention also provides a droplet assembly which is obtainable by a process for producing a droplet assembly as defined herein.

Process for Producing a Pre-Patterned Tissue Construct

The invention also provides a process for producing a pre-patterned tissue construct, the process comprising
producing a pre-patterned droplet assembly in a bulk medium by a process as described herein, or providing a pre-patterned droplet assembly as described herein, provided that, in the pre-patterned droplet assembly, the droplet medium comprises natural extracellular matrix material and biological cells;
gelling the natural extracellular matrix material to produce a pre-patterned tissue construct which comprises gelled natural extracellular matrix material and the biological cells; and
recovering the pre-patterned tissue construct from the bulk medium.

In the process of the invention for producing a pre-patterned tissue construct, “producing a pre-patterned droplet assembly in a bulk medium by a process as described herein” typically comprises: generating, in a bulk medium, a plurality of droplets, wherein each of said droplets comprises: (i) a droplet medium which comprises biological cells and a natural extracellular matrix material, and (ii) an outer layer of amphipathic molecules around the surface of the droplet medium, wherein the bulk medium and the droplet medium are immiscible, and contacting each of said droplets with another of said droplets to form a layer of said amphipathic molecules as an interface between contacting droplets. These steps may be as further defined herein for the process of the invention for producing a droplet assembly.

Alternatively, in the process of the invention for producing a pre-patterned tissue construct, “producing a pre-patterned droplet assembly in a bulk medium by a process as described herein” may be a process of the invention as defined herein for producing a droplet assembly using an apparatus for producing the droplet assembly. Thus, “producing a pre-patterned droplet assembly in a bulk medium by a process as described herein” may comprise: producing a droplet assembly using an apparatus for producing the droplet assembly, which droplet assembly comprises: a plurality of droplets, wherein each of said droplets comprises: (i) a droplet medium which comprises biological cells and a natural extracellular matrix material, and (ii) an outer layer of amphipathic molecules around the surface of the droplet medium; which apparatus comprises: at least one droplet generator wherein the droplet generator is suitable for generating droplets of a viscous droplet medium; a droplet receiving region which is moveable relative to the at least one droplet generator; a temperature controller; and a control unit, which control unit is adapted to control the dispensing of droplets from the at least one droplet generator and the movement of the droplet receiving region relative to the at least one droplet generator; wherein said droplet receiving region further comprises a bulk medium, wherein the bulk medium and the droplet medium are immiscible; which process comprises (a) a plurality of dispensing steps, wherein each dispensing step comprises dispensing a droplet of the droplet medium from a said droplet generator into the bulk medium, in the presence of amphipathic molecules, and thereby forming in the bulk medium a droplet which comprises (i) said droplet medium which comprises biological cells and a natural extracellular matrix material and (ii) an outer layer of amphipathic molecules around the surface of the droplet medium; and (b) moving the droplet receiving region relative to the at least one droplet generator, to control the relative positioning of the droplets in the bulk medium. These steps may be as further defined herein for the process of the invention for producing a droplet assembly using an apparatus for producing the droplet assembly.

In the process of the invention for producing a pre-patterned tissue construct, “providing a pre-patterned droplet assembly as described herein” may comprise providing a droplet assembly which is obtainable by the process of the invention for producing a droplet assembly, or providing a droplet assembly which is obtainable by the process of the invention for producing a droplet assembly using an apparatus for producing the droplet assembly.

Typically, however, in the process of the invention for producing a pre-patterned tissue construct, “providing a pre-patterned droplet assembly as described herein” comprises providing a droplet assembly of the invention as defined herein which comprises: a plurality of droplets in contact with one another, wherein each of said droplets comprises: (i) a droplet medium which comprises biological cells and a natural extracellular matrix material, and (ii) an outer layer of amphipathic molecules around the surface of the droplet medium, wherein each of said droplets contacts another of said droplets to form a layer of said amphipathic molecules as an interface between the contacting droplets. The provided droplet assembly may be as further defined herein for the droplet assembly of the invention.

The term “pre-patterned” is used herein to describe a droplet assembly in which the position, shape and size of the droplet assembly is pre-determined. The pre-determined position, shape and size of the droplet assembly may then be realised during the production of the droplet assembly, for instance in an additive process, such as a 3D printing process, which may be the process of the invention for producing a droplet assembly, or for instance the process of the invention for producing a droplet assembly using the apparatus of the invention.

The term “pre-patterned” may also describe droplet assembly in which two or more different kinds of droplets are present which are arranged into a particular pattern, for instance first and second regions of different droplets. The term “pre-patterned” is also used to refer to products originating from those droplet assembly, for instance products such as the tissue construct where the natural extracellular matrix material in the droplet assembly has been gelled, or the cultured tissue construct as described below. In such structures, the pre-patterning during the printing step can determine how and in what way biological cells present develop. Thus, pre-patterned structures may be useful for studying different interactions between different groups of cells, by allowing the precise positioning of the cells at the start of the experiment.

The natural extracellular matrix material may be any natural extracellular matrix material as described herein, for instance it may be matrigel. The biological cells may be any biological cells as described herein. The natural extracellular matrix material in the droplet medium is typically in liquid form and is capable of gelation on heating.

Typically, the droplet medium comprises biological cells at a density of at least 10⁴ cells/mL typically at least 10⁵ cells/mL preferably at least 10⁶ cells/mL. Typically, the droplet medium comprises biological cells at a density of up to 10⁸ cells/mL, for instance of up to 7.5×10⁷ cells/mL or up to 5×10⁷ cells/mL. For instance, the droplet medium may comprise biological cells at a density of from 1×10⁶ cells/mL to 7.5×10⁷ cells/mL, or from 1×10⁶ cells/mL to 5×10⁷ cells/mL, or from 1×10⁶ cells/mL to 4×10⁷ cells/mL. The cells may be any biological cells, for instance cells originating from any plant, animal, fungus or single-celled organism. Typically, the cells are mammalian cells, preferably human cells. For instance, the biological cells may be human liver cells, ovarian cells, breast cells or brain cells. The biological cells may be cancer cells. Typically the biological cells are selected from cell types found in soft tissue, for instance brain cells. Thus, the biological cells may comprise brain cells, typically human brain cells. The brain cells may comprise neural stem cells, neural progenitor cells, neurons, astrocytes, microglia or endothelial cells. Preferably, the brain cells comprise neural stem cells. The biological cells may comprise first and second types of biological cells which may be cells from different corticol layers, typically different corticol layers in the human brain. For instance, the biological cells of the first type may be biological cells from one corticol layer and the biological cells of the second type may be biological cells from another, different coritcol layer. Typically, the first type of biological cell may comprise neurons with or without astrocytes from specific cortical layer and the second type may comprise neurons with/without astrocytes from another specific cortical layer. There are six cortical layers in human brain that consist of different types of neurons. 3D printing may be used pattern these layers together to provide layered cortical tissue as mentioned in Example 1.

The natural extracellular matrix material is preferably matrigel. For instance, the droplet medium may comprise matrigel and the biological cells. It preferably comprises matrigel and mammalian cells, for instance matrigel and brain cells, typically matrigel and human brain cells. Typically, the natural extracellular matrix material is undiluted. The droplet medium may therefore comprise an undiluted natural extracellular matrix material and biological cells. Preferably the droplet medium comprises undiluted matrigel and biological cells.

Typically, the natural extracellular matrix material and the biological cells together form at least 80% by volume of the droplet medium, preferably at least 90% by volume of the droplet medium, more preferably at least 95% by volume of the droplet medium, for instance at least 99% by volume of the droplet medium. Typically, the droplet medium consists of the natural extracellular matrix material and the biological cells, preferably wherein the natural extracellular matrix material is matrigel.

The natural extracellular matrix material may comprise collagen.

Typically, gelling is induced by a change in temperature, for instance heating or cooling the droplet medium from the temperature at the time of generating a droplet. Preferably, the droplet medium is capable of gelation on heating.

Thus, typically, the step of gelling the natural extracellular matrix material comprises: increasing the temperature of the bulk medium. For instance, gelling the natural extracellular matrix material may comprise increasing the temperature of the bulk medium to a first temperature which is above said droplet printing temperature. The droplet printing temperature may be any droplet printing temperature as described herein. Typically, the droplet printing temperature is a temperature of less than 10° C., preferably a temperature of from 2° C. to 8° C., for instance at a temperature of about 5° C. Typically, the temperature of the bulk medium is increased to said first temperature, which is a temperature of from 10° C. to 30° C., preferably from 20° C. to 30° C., for instance about 25° C. The temperature of the bulk medium may be held at the first temperature for at least 10 minutes.

The temperature of the bulk medium may then be further increased to a second temperature which is higher than the first temperature, preferably wherein the second temperature is a temperature of from 30° C. to 40° C., preferably a temperature of from 35° C. to 40° C., for instance about 37° C. The temperature of the bulk medium may be held at the second temperature for at least 30 minutes, preferably at least 1 hour.

Generally, recovering the pre-patterned tissue construct from the bulk medium comprises transferring the tissue construct into an aqueous medium, preferably wherein the aqueous medium is cell culture medium. Typically, recovering the pre-patterned tissue construct from the bulk medium comprises (i) replacing at least part of the bulk medium with fresh bulk medium in order to reduce the concentration of amphipathic molecules in the bulk medium, and subsequently (ii) replacing the bulk medium with an aqueous medium, preferably wherein the aqueous medium is cell culture medium.

The invention also provides a pre-patterned tissue construct which is obtainable by a process of the invention for producing a pre-patterned tissue construct.

Process for Producing a Cultured Tissue Construct

The invention also provides a process for producing a cultured tissue construct comprising
preparing a pre-patterned tissue construct by the process of the invention for producing a pre-patterned tissue construct; and
culturing the biological cells in the pre-patterned tissue construct.

Culturing the biological cells typically means keeping the tissue construct under conditions in which cells expected to remain viable, in order to establish their behaviour. Thus, culturing the biological cells in the pre-patterned tissue construct typically comprises keeping the pre-patterned tissue construct in cell culture medium under conditions suitable for cell growth and/or cell differentiation. The cells are typically cultured for a duration of at least one day, preferably for at least one week, and optionally for a duration of up to 100 days.

Culturing the biological cells in the pre-patterned tissue construct may cause a reorganization of the cells in the tissue construct. Reorganization includes any change to the cells' position, number or morphology. Typically, reorganization of the cells in the tissue construct comprises cell differentiation, cell migration, growth of existing cells and/or genesis of new cells. The reorganization may approximate a natural process or it may be unnatural. Typically, for a natural process, the cells are provided with the nutrients and conditions needed for growth and development, but without the addition of any particular physical or chemical stimulus to influence the growth or behaviour of the cells. Additionally or alternatively, for a natural process, the cells may be positioned with respect to one another, in the pre-patterned tissue construct, in a way that mimics or approximates a natural structure in vivo. For an unnatural process, further physical or chemical stimuli may be added to study their effect on the growth and development of the cells. Additionally or alternatively, for an unnatural process, the cells may be positioned with respect to one another, in the pre-patterned tissue construct, in an unnatural way, i.e. in a way that is different from any natural structure in vivo for the cells in question.

Thus, culturing the biological cells in the pre-patterned tissue construct may comprise applying a physical or chemical stimulus to the cells in order to influence the reorganization. Typically, applying said physical or chemical stimulus comprises controlling the temperature of the cells or contacting the cells with a chemical. For instance, said stimulus may be the vectorial application of a growth factor, or the addition of a drug. Alternatively, culturing the biological cells in the pre-patterned tissue construct may comprises allowing the cells to reorganise naturally.

Reorganization of the cells in the tissue construct may comprise cell differentiation, cell migration, growth of existing cells and/or genesis of new cells.

The biological cells may be any biological cells as described herein. Typically the biological cells are selected from cell types found in soft tissue. Typically, the biological cells comprise brain cells, typically human brain cells, optionally wherein the brain cells comprise neural stem cells, neural progenitor cells, neurons, astrocytes, microglia or endothelial cells, wherein reorganization of the cells in the tissue construct comprises cell differentiation, cell migration, process outgrowth, astrogenesis, and/or axonal bundling (fasciculation).

The pre-patterned tissue construct may comprise (a) a first region which comprises biological cells of a first type and said gelled natural extracellular matrix material, and (b) a second region which comprises said gelled natural extracellular matrix material, optionally wherein the first region is adjacent the second region, or wherein the first region is an internal region and the second region is an external region which encases the internal region. Thus, the position of the first and second regions is determined during the printing process, and the impact of the initial position and density of cells on the development of the cells in culture can be observed.

The biological cells of the first type may be any biological cells as described herein. Typically the biological cells are selected from cell types found in soft tissue. Typically, the biological cells of the first type are brain cells, typically human brain cells, optionally wherein the biological cells of the first type comprise brain cells which are neural stem cells, neural progenitor cells, neurons, astrocytes, microglia or endothelial cells, preferably wherein the biological cells of the first type comprise neural stem cells or cortical neurons, more preferably neural stem cells. Reorganization of the cells in the tissue construct may comprise differentiation of the biological cells of the first type, migration of the biological cells of the first type optionally from the first region to the second region, process outgrowth from the biological cells of the first type optionally from the first region into the second region, astrogenesis from the biological cells of the first type, and/or axonal bundling (fasciculation).

The second region may comprise said gelled natural extracellular matrix material and biological cells of a second type which is different from the first type. Alternatively, the second region may comprise gelled natural extracellular matrix material and no biological cells.

The biological cells of the second type may be any biological cells as described herein. Typically the biological cells are selected from cell types found in soft tissue.

Typically, the biological cells of the second type are a second type of brain cell, optionally wherein the biological cells of the second type comprise brain cells of a different type selected from neural stem cells, neural progenitor cells, neurons, astrocytes, microglia or endothelial cells, optionally wherein the biological cells of the first type comprise astrocytes. Reorganization of the cells in the tissue construct may further comprises differentiation of the biological cells of the second type, migration of the biological cells of the second type optionally from the second region to the first region, process outgrowth from the biological cells of the second type optionally from the second region into the first region, astrogenesis from the biological cells of the second type, and/or axonal bundling (fasciculation).

Thus, the reorganisation may comprise:
differentiation of neural stem cells;
process outgrowth from neural stem cells;
migration of neural stem cells;
differentiation of neural stem cells into astrocytes;
non-migration of astrocytes;
astrocyte-assisted axonal bundling (fasciculation); and/or
neuronal differentiation; and/or
migration of neurons, for instance migration of cortical neurons.

The first and second types of biological cells may be cells from different corticol layers, typically different corticol layers in the human brain. For instance, the biological cells of the first type may be biological cells from one corticol layer and the biological cells of the second type may be biological cells from another, different coritcol layer. Typically, the first type of biological cell may comprise neurons with or without astrocytes from specific cortical layer and the second type may comprise neurons with/without astrocytes from another specific cortical layer. There are six cortical layers in human brain that consist of different types of neurons. 3D printing may be used pattern these layers together to provide layered cortical tissue as mentioned in Example 1.

In the process for producing a cultured tissue construct it may be that:
the biological cells of the first type may comprise neural stem cells and said reorganisation comprises process outgrowth from the neural stem cells (optionally from the first region to the second region), differentiation of the neural stem cells (optionally into differentiated neurons), migration of the neural stem cells (optionally from the first region to the second region), astrogenesis, or a combination of two or more thereof, or
the biological cells of the first type may comprise cortical neurons and said reorganisation comprises migration of cortical neurons, optionally from the first region to the second region.

In the process for producing a cultured tissue construct it may be that:
the biological cells of the first type may comprise neural stem cells and said reorganisation comprises process outgrowth from the neural stem cells (optionally from the first region to the second region), differentiation of the neural stem cells (optionally into differentiated neurons), migration of the neural stem cells (optionally from the first region to the second region), astrogenesis, or a combination of two or more thereof, and
the biological cells of the second type may comprise astrocytes and said reorganisation further comprises astrocytes-assisted axonal bundling (fasciculation) in the second region.

The process may further comprise recovering the cultured tissue construct following said reorganisation.

The invention also provides a cultured tissue construct which is obtainable by the process of the invention for producing a cultured tissue construct.

Droplet Assembly of Amphipathic-Connected Droplets of Natural ECM and Cells

The invention also provides a droplet assembly comprising: a plurality of droplets in contact with one another, wherein each of said droplets comprises: (i) a droplet medium which comprises biological cells and a natural extracellular matrix material, and (ii) an outer layer of amphipathic molecules around the surface of the droplet medium, wherein each of said droplets contacts another of said droplets to form a layer of said amphipathic molecules as an interface between the contacting droplets.

The number of droplets in said plurality may be greater than 10, preferably greater than 100, optionally greater than 1000, for example greater than 10000. For instance, the number of droplets in said plurality of droplets may be greater than 20000, greater than 50000 or greater than 100000. The droplet assembly is typically an extended two or three-dimensional network of droplets, preferably an extended three-dimensional network of droplets. The droplet assembly may comprise at least one layer of droplets, wherein each of said droplets comprises (i) said droplet medium and (ii) an outer layer of amphipathic molecules around the surface of the droplet medium, and wherein each droplet in the layer contacts at least one other droplet in the layer to form a layer of said amphipathic molecules as an interface between contacting droplets. The droplet assembly may preferably comprise a plurality of layers of said droplets, wherein each layer is disposed adjacent to another layer, so that droplets in a layer contact droplets in an adjacent layer to form layers of amphipathic molecules as interfaces between the contacting droplets. For instance the layers of droplets may be stacked on top of each other to form a three-dimensional structure. The number of droplets may be greater than 10 and is preferably greater than 100, optionally greater than 1000, for example greater than 10000. For instance, the number of droplets in said plurality of droplets may be greater than 20000, greater than 50000 or greater than 100000.

Typically, each layer of said amphipathic molecules which is an interface between contacting droplets is a bilayer of said amphipathic molecules.

The amphipathic molecules may be any amphipathic molecules as described herein. Typically, the amphipathic molecules comprise one or more glycerophospholipids, optionally wherein the amphipathic molecules comprise 1,2-diphytanoyl-sn-glycero-3-phosphocholine (DPhPC).

Typically, each of said droplets has a diameter of equal to or less than 1 mm, optionally equal to or less than 250 μm, equal to or less than 150 μm, equal to or less than 90 μm, or equal to or less than 40 μm, preferably wherein each of said droplets has a diameter of from 10 μm to 250 μm. Each of said droplets may have a volume of equal to or less than 2 μL, preferably wherein each of said droplets has a volume of equal to or less than 100 nL, equal to or less than 350 pL, equal to or less than 150 pL, or equal to or less than 45 pL, for instance from 0.001 nL (1 pL) to 100 nL, or from 0.001 nL (1 pL) to 500 pL.

The natural extracellular matrix material may be any natural extracellular matrix material as described herein, for instance it may be matrigel. The biological cells may be any biological cells as described herein. The natural extracellular matrix material in the droplet medium is typically in liquid form and is capable of gelation on heating.

Typically, the droplet medium comprises biological cells at a density of at least 10⁴ cells/mL typically at least 10⁷ cells/mL preferably at least 10⁶ cells/mL. Typically, the droplet medium comprises biological cells at a density of up to 10⁸ cells/mL, for instance of up to 7.5×10⁷ cells/mL or up to 5×10⁷ cells/mL. For instance, the droplet medium may comprise biological cells at a density of from 1×10⁶ cells/mL to 7.5×10⁷ cells/mL, or from 1×10⁶ cells/mL to 5×10⁷ cells/mL, or from 1×10⁶ cells/mL to 4×10⁷ cells/mL. The cells may be any biological cells, for instance cells originating from any plant, animal, fungus or single-celled organism. Typically, the cells are mammalian cells, preferably human cells. For instance, the biological cells may be human liver cells, ovarian cells, breast cells or brain cells. The biological cells may be cancer cells. Typically the biological cells are selected from cell types found in soft tissue, for instance brain cells. Thus, the biological cells may comprise brain cells, typically human brain cells. The brain cells may comprise neural stem cells, neural progenitor cells, neurons, astrocytes, microglia or endothelial cells. Preferably, the brain cells comprise neural stem cells.

The natural extracellular matrix material is preferably matrigel. For instance, the droplet medium may comprise matrigel and the biological cells. It preferably comprises matrigel and mammalian cells, for instance matrigel and brain cells, typically matrigel and human brain cells. Typically, the natural extracellular matrix material is undiluted. The droplet medium may therefore comprise an undiluted natural extracellular matrix material and biological cells. Preferably the droplet medium comprises undiluted matrigel and biological cells. The droplet medium may comprise collagen, fibronectin, hyaluronic acid and/or laminin.

Typically, the natural extracellular matrix material and the biological cells together form at least 80% by volume of the droplet medium, preferably at least 90% by volume of the droplet medium, more preferably at least 95% by volume of the droplet medium, for instance at least 99% by volume of the droplet medium. Typically, the droplet medium consists of the natural extracellular matrix material and the biological cells, preferably wherein the natural extracellular matrix material is matrigel.

The droplet assembly is typically disposed in a bulk medium wherein the bulk medium and the droplet medium are immiscible. The bulk medium may be any bulk medium as described herein. Typically, the bulk medium is a hydrophobic medium, for instance an oil or mixture of oils. Preferably, the bulk medium comprises silicone oil and a hydrocarbon, optionally wherein the hydrocarbon is a straight-chained, unsubstituted C₈₋₁₅ alkane, and preferably wherein the hydrocarbon is undecane. Typically, the ratio by volume of the hydrocarbon and the silicone oil is selected such that the density of the hydrophobic medium matches that of the droplet medium at the printing temperature.

The bulk medium may comprise or consist of a naturally occurring oil, for instance sunflower oil. The bulk medium may comprise or consist of the primary constituent of naturally occurring oil, for instance, the bulk medium may comprise or consist of glyceryl trioleate and/or glyceryl trilinoleate.

Preferably, the droplet assembly does not comprise an extraneous scaffold material.

Typically, the droplets in the droplet assembly are arranged in a predetermined shape or in an ordered pattern. The droplet assembly may comprise: a first region of said droplets, wherein each droplet in the first region contacts at least one other droplet in the first region to form a layer (e.g. bilayer) of said amphipathic molecules as an interface between contacting droplets; and a second region of said droplets, wherein each droplet in the second region contacts at least one other droplet in the second region to form a layer (e.g. bilayer) of said amphipathic molecules as an interface between contacting droplets, wherein the droplet medium of the droplets in the first region is a first droplet medium and the droplet medium of the droplets in the second region is a second droplet medium wherein the first droplet medium and the second droplet medium are different. Typically, the first droplet medium comprises biological cells of a first type and the second droplet medium comprises biological cells of a second type which is different from the first type. Alternatively, the first droplet medium may comprise biological cells of a first type and the second droplet medium does not comprise biological cells.

The biological cells of the first type and the biological cells of the second type may be any types of biological cells as described herein. Typically the biological cells are selected from cell types found in soft tissue. Typically, the biological cells of the first type and the biological cells of the second type are different types of brain cells, typically human brain cells, optionally wherein the biological cells of the first type comprise one of neural stem cells, neural progenitor cells, neurons, astrocytes, microglia and endothelial cells, and the biological cells of the second type, where present, comprise a different one of neural stem cells, neural progenitor cells, neurons, astrocytes, microglia and endothelial cells. Preferably, the biological cells of the first type comprise neural stem cells or cortical neurons and optionally the biological cells of the second type, where present, comprise astrocytes. The first and second types of biological cells may be cells from different corticol layers, typically different corticol layers in the human brain. For instance, the biological cells of the first type may be biological cells from one corticol layer and the biological cells of the second type may be biological cells from another, different coritcol layer. Typically, the first type of biological cell may comprise neurons with or without astrocytes from specific cortical layer and the second type may comprise neurons with/without astrocytes from another specific cortical layer. There are six cortical layers in human brain that consist of different types of neurons. 3D printing may be used pattern these layers together to provide layered cortical tissue as mentioned in Example 1.

The droplets of the first region and the droplets of the second region may be arranged in an ordered pattern, optionally wherein the first region is adjacent the second region, or wherein the first region is an internal region and the second region is an external region which encases the internal region.

Pre-Patterned Tissue Construct of Cells in Gelled Droplets Ready for Culturing

The invention also provides a pre-patterned tissue construct comprising a plurality of gelled droplets in contact with one another, wherein each of said droplets comprises a droplet medium which comprises biological cells and a gelled natural extracellular matrix material, and wherein each of said droplets is adhered to another of said droplets by the gelled natural extracellular matrix material.

Typically, the number of gelled droplets in said plurality is greater than 10, preferably greater than 100, optionally greater than 1000, for example greater than 10000. For instance, the number of gelled droplets in said plurality of gelled droplets may be greater than 20000, greater than 50000 or greater than 100000. The pre-patterned tissue construct is typically an extended two or three-dimensional network of gelled droplets, preferably an extended three-dimensional network of gelled droplets. The pre-patterned tissue construct may comprise at least one layer of gelled droplets, wherein each of said droplets comprises said gelled natural extracellular matrix material, and wherein each droplet in the layer contacts at least one other droplet in the layer. The pre-patterned tissue construct assembly may preferably comprise a plurality of layers of said gelled droplets, wherein each layer is disposed adjacent to another layer, so that droplets in a layer contact droplets in an adjacent layer. For instance the layers of gelled droplets may be stacked on top of each other to form a three-dimensional structure. The number of droplets is preferably greater than 100, optionally greater than 1000, for example greater than 10000. For instance, the number of gelled droplets in said plurality of gelled droplets may be greater than 20000, greater than 50000 or greater than 100000.

Typically, the plurality of gelled droplets in contact with one another is a plurality of printed gelled droplets in contact with one another.

The biological cells may be any biological cells as described herein. Typically, the biological cells comprise brain cells, typically human brain cells, optionally wherein the brain cells comprise neural stem cells, neural progenitor cells, neurons, astrocytes, microglia or endothelial cells, preferably wherein the brain cells comprise neural stem cells. Typically, the droplet medium comprises biological cells at a density of at least 10⁴ cells/mL typically at least 10⁷ cells/mL preferably at least 10⁶ cells/mL. Typically, the droplet medium comprises biological cells at a density of up to 10⁸ cells/mL, for instance of up to 7.5×10⁷ cells/mL or up to 5×10⁷ cells/mL. For instance, the droplet medium may comprise biological cells at a density of from 1×10⁶ cells/mL to 7.5×10⁷ cells/mL, or from 1×10⁶ cells/mL to 5×10⁷ cells/mL, or from 1×10⁶ cells/mL to 4×10⁷ cells/mL.

The gelled natural extracellular matrix material may be any gelled natural extracellular matrix material as described herein. The gelled natural extracellular matrix material is preferably gelled matrigel. The gelled natural extracellular matrix material and the biological cells may together form at least 80% by volume of the droplet medium, preferably at least 90% by volume of the droplet medium, more preferably at least 95% by volume of the droplet medium, optionally at least 99% by volume of the droplet medium. For instance, the droplet medium may consist of the biological cells and the gelled natural extracellular matrix material, preferably wherein the droplet medium consists of the biological cells and gelled matrigel. The droplet medium may comprise of biological cells, gelled natural extracellular matrix material and at least one additive selected from collagen, fibronectin, hyaluronic acid and laminin. The droplet medium may consist of biological cells, gelled natural extracellular matrix material and at least one additive selected from collagen, fibronectin, hyaluronic acid and laminin.

Alternatively, the gelled natural extracellular matrix material may comprise, or consist of, collagen.

Typically, the pre-patterned tissue construct does not comprise an extraneous scaffold material.

Typically, the droplets are arranged in a predetermined shape or in an ordered pattern. For instance, the pre-patterned tissue construct may comprise: a first region of said droplets, wherein each droplet in the first region is adhered to at least one other droplet in the first region; and a second region of said droplets, wherein each droplet in the second region is adhered to at least one other droplet in the second region, wherein the droplet medium of the droplets in the first region is a first droplet medium and the droplet medium of the droplets in the second region is a second droplet medium wherein the first droplet medium and the second droplet medium are different. Typically, the first droplet medium comprises biological cells of a first type and the second droplet medium comprises biological cells of a second type which is different from the first type. Alternatively, the first droplet medium may comprise biological cells of a first type and the second droplet medium does not comprise biological cells.

The biological cells of the first type and the biological cells of the second type may be any types of biological cells as described herein. Typically the biological cells are selected from cell types found in soft tissue. Typically, the biological cells of the first type and the biological cells of the second type are different types of brain cells, typically human brain cells, optionally wherein the biological cells of the first type comprise one of neural stem cells, neural progenitor cells, neurons, astrocytes, microglia and endothelial cells, and the biological cells of the second type, where present, comprise a different one of neural stem cells, neural progenitor cells, neurons, astrocytes, microglia and endothelial cells. Preferably, the biological cells of the first type comprise neural stem cells or cortical neurons and optionally wherein the biological cells of the second type, where present, comprise astrocytes. The first and second types of biological cells may be cells from different corticol layers, typically different corticol layers in the human brain. For instance, the biological cells of the first type may be biological cells from one corticol layer and the biological cells of the second type may be biological cells from another, different coritcol layer. Typically, the first type of biological cell may comprise neurons with or without astrocytes from specific cortical layer and the second type may comprise neurons with/without astrocytes from another specific cortical layer. There are six cortical layers in human brain that consist of different types of neurons.

The droplets of the first region and the droplets of the second region may be arranged in a two dimensional pattern or a three-dimensional pattern, preferably in a three dimensional pattern. A three-dimensional pattern typically comprises a network of droplets that is more than one droplet across in each of the three dimensions, for instance which comprises multiple stacked layers of droplets. The term “ordered pattern”, as used herein, refers to an arrangement in which the droplets are not simply positioned at random. Typically, the droplets of the first region and the droplets of the second region are arranged in an ordered pattern. For instance, the first region may be adjacent the second region, or the first region may be an internal region and the second region is an external region which encases the internal region.

The pre-patterned tissue construct may be disposed in an aqueous medium, preferably wherein the aqueous medium is cell culture medium. The pre-patterned tissue construct may be disposed in a bulk medium wherein the bulk medium and the droplet medium are immiscible. The bulk medium may be any bulk medium as described herein, for instance any hydrophobic medium as described herein.

Cultured Tissue Construct

The invention provides a cultured tissue construct which comprises a natural extracellular matrix material and biological cells, wherein the cultured tissue construct is obtainable by providing a pre-patterned tissue construct of the invention as defined herein and culturing the biological cells in the pre-patterned tissue construct.

Providing a pre-patterned tissue construct of the invention as defined herein comprises providing a pre-patterned tissue construct comprising a plurality of gelled droplets in contact with one another, wherein each of said droplets comprises a droplet medium which comprises biological cells and a gelled natural extracellular matrix material, and wherein each of said droplets is adhered to another of said droplets by the gelled natural extracellular matrix material. The pre-patterned tissue construct that is provided may be as further defined above for the pre-patterned tissue construct of the invention, or it may be as further defined above for the pre-patterned tissue construct which is obtainable by the process of the invention for producing a pre-patterned tissue construct.

Culturing the biological cells in the pre-patterned tissue construct typically causes a reorganization of the cells in the pre-patterned tissue construct to produce the cultured tissue construct. Reorganisation includes any change to the cells' position, number or morphology. Typically, reorganization of the cells in the tissue construct comprises cell differentiation, cell migration, growth of existing cells and/or genesis of new cells. The reorganization may approximate a natural process or may be unnatural. Typically, for a natural process, the cells are provided with the nutrients and conditions needed for growth and development, but without the addition any particular physical or chemical stimulus to influence the growth or behaviour of the cells. Additionally or alternatively, for a natural process, the cells may be positioned with respect to one another, in the pre-patterned tissue construct, in a way that mimics or approximates a natural structure in vivo. For an unnatural process, further physical or chemical stimuli may be added to study their effect on the growth and development of the cells. Additionally or alternatively, for an unnatural process, the cells may be positioned with respect to one another, in the pre-patterned tissue construct, in an unnatural way, i.e. in a way that is different from any natural structure in vivo for the cells in question.

Thus, culturing the biological cells in the pre-patterned tissue construct may comprise applying a physical or chemical stimulus to the cells in order to influence the reorganization. Typically, applying said physical or chemical stimulus comprises controlling the temperature of the cells or contacting the cells with a chemical. For instance, said stimulus may be the vectorial application of a growth factor, or the addition of a drug. Alternatively, culturing the biological cells in the pre-patterned tissue construct may comprises allowing the cells to reorganise naturally.

Typically, the reorganization of the cells in the pre-patterned tissue construct to produce the cultured tissue construct is influenced by a pattern in which the cells were arranged in the pre-patterned tissue construct. For instance, the pattern in which the cells were arranged in the pre-patterned tissue construct may be designed to encourage cell growth or migration to a particular part of the tissue construct, or to encourage interaction between two or more different cell types.

Typically, culturing the biological cells in the pre-patterned tissue construct to produce the cultured tissue construct comprises keeping the pre-patterned tissue construct in cell culture medium under conditions suitable for cell growth and/or cell differentiation, optionally for a duration of at least one day, preferably for at least one week, and optionally for a duration of up to 100 days.

The biological cells may be any biological cells as described herein. The cells may be any biological cells, for instance cells originating from any plant, animal, fungus or single-celled organism. Typically, the cells are mammalian cells, preferably human cells. For instance, the biological cells may be human liver cells, ovarian cells, breast cells or brain cells. The biological cells may be cancer cells. Typically the biological cells are selected from cell types found in soft tissue. Typically, the biological cells comprise brain cells, typically human brain cells, optionally wherein the brain cells comprise neural stem cells, neural progenitor cells, neurons, astrocytes, microglia or endothelial cells. Preferably, the brain cells comprise neural stem cells. Typically, reorganization of the cells in the tissue construct comprises cell differentiation, cell migration, process outgrowth, astrogenesis, and/or axonal bundling (fasciculation).

The cultured tissue construct may comprises (a) a first region which comprises biological cells of a first type and a natural extracellular matrix material, and (b) a second region which comprises said natural extracellular matrix material, optionally wherein the first region is adjacent the second region, or wherein the first region is an internal region and the second region is an external region which encases the internal region.

The biological cells of the first type may be any biological cells as described herein. Typically the biological cells are selected from cell types found in soft tissue. For instance, the biological cells of the first type are brain cells, typically human brain cells, optionally wherein the biological cells of the first type comprise brain cells which are neural stem cells, neural progenitor cells, neurons, astrocytes, microglia or endothelial cells, preferably wherein the biological cells of the first type comprise neural stem cells or cortical neurons, more preferably neural stem cells. Reorganization of the cells in the pre-patterned tissue construct to produce the cultured tissue construct may comprise differentiation of the biological cells of the first type, migration of the biological cells of the first type optionally from the first region to the second region, process outgrowth from the biological cells of the first type optionally from the first region into the second region, astrogenesis from the biological cells of the first type, and/or axonal bundling (fasciculation).

Typically, the second region comprises said natural extracellular matrix material and biological cells of a second type which is different from the first type. Alternatively, the second region may comprise said natural extracellular matrix material and no biological cells.

The biological cells of the second type may be any biological cells as described herein. Typically the biological cells are selected from cell types found in soft tissue. For instance, the biological cells of the second type may be a second type of brain cell, optionally wherein the biological cells of the second type comprise brain cells of a different type selected from neural stem cells, neural progenitor cells, neurons, astrocytes, microglia or endothelial cells, optionally wherein the biological cells of the first type comprise astrocytes. Reorganization of the cells in the pre-patterned tissue to produce the cultured tissue construct typically comprises differentiation of the biological cells of the second type, migration of the biological cells of the second type optionally from the second region to the first region, process outgrowth from the biological cells of the second type optionally from the second region into the first region, astrogenesis from the biological cells of the second type, and/or axonal bundling (fasciculation).

Reorganization of the cells in the pre-patterned tissue to produce the cultured tissue construct may comprise:
differentiation of neural stem cells;
process outgrowth from neural stem cells;
migration of neural stem cells;
differentiation of neural stem cells into astrocytes;
non-migration of astrocytes;
astrocyte-assisted axonal bundling (fasciculation); and/or
neuronal differentiation; and/or
migration of neurons, for instance migration of cortical neurons.

The first and second types of biological cells may be cells from different corticol layers, typically different corticol layers in the human brain. For instance, the biological cells of the first type may be biological cells from one corticol layer and the biological cells of the second type may be biological cells from another, different coritcol layer. Typically, the first type of biological cell may comprise neurons with or without astrocytes from specific cortical layer and the second type may comprise neurons with/without astrocytes from another specific cortical layer. There are six cortical layers in human brain that consist of different types of neurons.

Typically, in the cultured tissue construct according of the invention:
the biological cells of the first type comprise neural stem cells and said reorganization of the cells in the pre-patterned tissue to produce the cultured tissue construct comprises process outgrowth from the neural stem cells (optionally from the first region to the second region), differentiation of the neural stem cells (optionally into differentiated neurons), migration of the neural stem cells (optionally from the first region to the second region), astrogenesis, or a combination of two or more thereof, or
the biological cells of the first type comprise cortical neurons and said reorganization of the cells in the pre-patterned tissue to produce the cultured tissue construct comprises migration of cortical neurons, optionally from the first region to the second region.

Typically, in the cultured tissue construct according of the invention:
the biological cells of the first type comprise neural stem cells and said reorganization of the cells in the pre-patterned tissue to produce the cultured tissue construct comprises process outgrowth from the neural stem cells (optionally from the first region to the second region), differentiation of the neural stem cells (optionally into differentiated neurons), migration of the neural stem cells (optionally from the first region to the second region), astrogenesis, or a combination of two or more thereof, and
the biological cells of the second type comprise astrocytes and said reorganisation further comprises astrocytes-assisted axonal bundling (fasciculation) in the second region.

Droplet Arrays as-Printed

The invention also provides a droplet array which comprises a plurality of elements spaced apart from one another on a substrate in a bulk medium, wherein each element comprises at least one droplet which comprises a droplet medium which comprises one or more biological cells and a natural extracellular matrix material, wherein the bulk medium and the droplet medium are immiscible.

Often, the droplet array comprises amphipathic molecules. The amphipathic molecules may be any amphipathic molecules as described herein. Thus, each element in the array may comprise at least one droplet which comprises (i) a droplet medium which comprises one or more biological cells and a natural extracellular matrix material and (ii) an outer layer of amphipathic molecules. For instance, it may be that every droplet in each element in the array comprises (i) a droplet medium which comprises one or more biological cells and a natural extracellular matrix material and (ii) an outer layer of amphipathic molecules. Alternatively, it may be that the array comprises one or more droplets which do not comprise amphipathic molecules; those one or more droplets may consist only of the droplet medium.

At least one element in the array may comprise a single droplet. Typically, at least one of the elements, optionally each of the elements, comprises an individual droplet which comprises said droplet medium, wherein the individual droplet is not in contact with any other droplet in the droplet array. For instance, at least one of the elements, optionally each of the elements, comprises an individual droplet which comprises (i) said droplet medium and (ii) an outer layer of amphipathic molecules, wherein the individual droplet is not in contact with any other droplet in the droplet array. Typically, the outer layer of amphipathic molecules on each droplet in the array is not in contact with the outer layer of amphipathic molecules on any other droplet in the array. Thus, the droplets in the array may be spaced apart so that no layer/bilayer forms between droplets.

At least one element in the array may comprise multiple droplets. For instance, at least one element in the array may comprise two or more droplets or three or more droplets.

Thus, at least one of the elements, optionally each of the elements, may comprise a pair of droplets, wherein each droplet in the pair comprises said droplet medium. Optionally, the pair of droplets is not in contact with any other droplet in the droplet array. For instance, at least one of the elements, optionally each of the elements, may comprise a pair of droplets, wherein each droplet in the pair comprises said droplet medium, wherein the pair of droplets is not in contact with any other droplet in the droplet array, and wherein the two droplets in the pair contact one another. For instance, at least one of the elements, optionally each of the elements, may comprise a pair of droplets, wherein each droplet in the pair comprises: (i) said droplet medium, and (ii) an outer layer of amphipathic molecules around the surface of the droplet medium; optionally, the pair of droplets is not in contact with any other droplet in the droplet array; furthermore, optionally, the two droplets in the pair contact one another to form a layer of said amphipathic molecules as an interface between the contacting droplets. Thus, at least one of the elements, optionally each of the elements, may comprise a pair of droplets, wherein each droplet in the pair comprises: (i) said droplet medium, and (ii) an outer layer of amphipathic molecules around the surface of the droplet medium, wherein the pair of droplets is not in contact with any other droplet in the droplet array, and wherein the two droplets in the pair contact one another to form a layer (e.g. a bilayer) of said amphipathic molecules as an interface between the contacting droplets.

Typically, one droplet in the or each pair of droplets contains a first type of biological cell and the other droplet in the or each pair of droplets contains a second type of biological cell which is different from the first type. The first type of biological cell may be any biological cell as described herein. The second type of biological cell may be any biological cell as described herein. Typically, the first type of biological cell and the second type of biological cell are selected from mammalian cells, preferably human cells. For instance, the first type of biological cell and the second type of biological cell may be selected from human liver cells, ovarian cells, breast cells or brain cells. The first type of biological cell and the second type of biological cell may be different types of cancer cells. Typically the biological cells are selected from cell types found in soft tissue. Thus, typically, the first type of biological cell and the second type of biological cell are selected from brain cells, typically human brain cells, optionally wherein the first type of biological cell and the second type of biological cell are brain cells selected from neural stem cells, neural progenitor cells, neurons, astrocytes, microglia and endothelial cells.

Both of the droplets in the or each pair may contact the substrate. Alternatively, only a first droplet in the or each pair contacts the substrate and the other droplet in the or each pair is disposed on top of the first droplet and does not contact the substrate.

At least one of the elements in the array, optionally each of the elements, may comprise a group of at least three droplets, wherein each of the at least three droplets comprises said droplet medium. At least one of the elements, optionally each of the elements, may comprise a group of at least three droplets, wherein each of the at least three droplets comprises said droplet medium, wherein the group of at least three droplets is not in contact with any other droplet in the droplet array, and wherein each of the at least three droplets in the group contacts another of the at least three droplets in the group. At least one of the elements, optionally each of the elements, may comprise a group of at least three droplets, wherein each of the at least three droplets comprises: (i) said droplet medium, and optionally (ii) an outer layer of amphipathic molecules around the surface of the droplet medium, optionally wherein the group of at least three droplets is not in contact with any other droplet in the droplet array, and optionally wherein each of the at least three droplets in the group contacts another of the at least three droplets in the group to form a layer of said amphipathic molecules as an interface between the contacting droplets. At least one of the elements, optionally each of the elements, may comprise a group of at least three droplets, wherein each of the at least three droplets comprises: (i) said droplet medium, and optionally (ii) an outer layer of amphipathic molecules around the surface of the droplet medium, wherein the group of at least three droplets is not in contact with any other droplet in the droplet array, and wherein each of the at least three droplets in the group contacts another of the at least three droplets in the group to form a layer of said amphipathic molecules as an interface between the contacting droplets.

Typically, one droplet in the or each group of at least three droplets contains a first type of biological cell and another droplet in the or each group of at least three droplets contains a second type of biological cell which is different from the first type. The first type of biological cell may be any biological cell as described herein. The second type of biological cell may be any biological cell as described herein. Typically the biological cells are selected from cell types found in soft tissue. Typically, the cells are mammalian cells, preferably human cells. For instance, the biological cells may be human liver cells, ovarian cells, breast cells or brain cells. The biological cells may be cancer cells. For instance, the first type of biological cell and the second type of biological cell may be selected from brain cells, typically human brain cells, optionally wherein the first type of biological cell and the second type of biological cell are brain cells selected from neural stem cells, neural progenitor cells, neurons, astrocytes, microglia and endothelial cells.

The at least three droplets in the or each group may be arranged in a micropattern. For instance, the at least three droplets in the or each group may be arranged to form a row of the least three droplets along the substrate or to form a tower of the least three droplets protruding up from the substrate.

The bulk medium may be any bulk medium as described herein. The amphipathic may be any amphipathic molecules as described herein. Typically the amphipathic molecules comprise one or more glycerophospholipids, preferably the amphipathic molecules comprise 1,2-diphytanoyl-sn-glycero-3-phosphocholine (DPhPC).

Each of said droplets in the array may have a diameter of equal to or less than 1 mm; optionally equal to or less than 250 μm, equal to or less than 150 μm, equal to or less than 90 μm, or equal to or less than 40 μm; preferably wherein each of said droplets has a diameter of from 10 μm to 250 μm. Each of said droplets may have a volume of equal to or less than 2 μL; preferably wherein each of said droplets has a volume of equal to or less than 100 nL, equal to or less than 350 pL, equal to or less than 150 pL, or equal to or less than 45 pL; for instance from 0.001 nL (1 pL) to 100 nL, or from 0.001 nL (1 pL) to 500 pL.

The printing processes described herein allow a high degree of control over the droplet size. By controlling the concentration of the cells in the droplet medium, the number of cells in the droplets produced can be controlled. Thus, the present invention makes it possible to produce arrays in of droplets wherein each droplet contains a small number of cells. Typically, at least 20% of the droplets in the array, preferably at least 30% of the droplets in the array, more preferably at least 40% of the droplets in the array, optionally at least 70% of the droplets in the array, for instance all of the droplets in the array, each comprise a specific number, c, of biological cells. Typically, c is an integer of from 1 to 20. Preferably c is from 1 to 10, more preferably wherein c is from 1 to 5, for instance wherein c may be 1.

The one or more biological cells may be any biological cells as described herein. Typically, the biological cells are selected from brain cells, typically human brain cells, optionally wherein the brain cells are selected from neural stem cells, neural progenitor cells, neurons, astrocytes, microglia and endothelial cells, preferably wherein the brain cells are neural stem cells or astrocytes.

The droplets may comprise a mixture of biological cell types. For instance, a portion of the droplets in the array may comprise more than one type of biological cell. For instance, a portion of the droplets in the array may comprise two types of biological cells. Typically, at least 5% of the droplets in the array, preferably at least 10% of the droplets in the array, more preferably at least 15% of the droplets in the array, optionally at least 50% of the droplets in the array, for instance all of the droplets in the array, each comprise a first type of biological cell and a second type of biological cell which is different from the first type of biological cell. The first type of biological cell may be any biological cell as described herein. The second type of biological cell may be any biological cell as described herein. Typically the biological cells are selected from cell types found in soft tissue. Typically, the first type of biological cell and the second type of biological cell are selected from mammalian cells, preferably human cells. For instance, the first type of biological cell and the second type of biological cell may be selected from human liver cells, ovarian cells, breast cells or brain cells. The biological cells may be cancer cells. Typically, the first type of biological cell and the second type of biological cell are selected from brain cells, typically human brain cells. For instance, the first type of biological cell and the second type of biological cell may be brain cells selected from neural stem cells, neural progenitor cells, neurons, astrocytes, microglia and endothelial cells.

The natural extracellular matrix material may be any natural extracellular matrix material as described herein. Typically, the natural extracellular matrix material is matrigel. The natural extracellular matrix material may comprise collagen. Typically, the natural extracellular matrix material and the biological cells together form at least 80% by volume of the droplet medium, preferably at least 90% by volume of the droplet medium, more preferably at least 95% by volume, for instance at least 99% by volume, of the droplet medium. Typically, the droplet medium comprises an undiluted natural extracellular matrix material and one or more biological cells. Preferably, the droplet medium comprises undiluted matrigel and one or more biological cells. The droplet medium may consist of a natural extracellular matrix material and one or more biological cells. Preferably, the natural extracellular matrix material is matrigel. Typically, the natural extracellular matrix material consists of matrigel.

The droplet medium may be in a liquid state. Alternatively, the droplet medium may have been subjected to a gelling step, for instance a step in which the temperature is increased to gel the natural extracellular matrix material. Thus, the droplet medium may be in the form of a gel or in the solid state.

Typically, the number of elements in the array is greater than 10, preferably greater than 100. The number of droplets in the array is greater than 10, preferably greater than 100, optionally greater than 1000, for example greater than 10000. In this case the number of droplets includes all droplets making up each individual element in the array, in the situation where each element comprises multiple droplets.

The substrate may be any suitable substrate. The substrate typically comprises a polymer or glass. Typically, the substrate comprises a polymer, such as poly(methyl methacrylate) or polystyrene. The substrate typically is a polymer or glass. Typically, the substrate is a polymer, such as poly(methyl methacrylate) or polystyrene. The substrate may comprise a plurality of microwells. Each element in the array may be disposed in a microwell on the substrate.

Gelled Droplet Array

The invention provides a droplet array which comprises a plurality of elements spaced apart from one another on a substrate, wherein each element comprises at least one gelled droplet, wherein each gelled droplet comprises a droplet medium which comprises one or more biological cells and a gelled natural extracellular matrix material.

At least one element may comprise a single gelled droplet. Thus, typically, at least one of the elements, optionally each of the elements, comprises an individual gelled droplet which comprises said droplet medium wherein the individual gelled droplet is not in contact with any other gelled droplet in the droplet array.

At least one element in the array may comprise multiple gelled droplets. For instance, at least one element in the array may comprise two or more gelled droplets or three or more gelled droplets. Typically, at least one of the elements, optionally each of the elements, comprises a pair of gelled droplets, wherein each gelled droplet in the pair comprises said droplet medium. Typically, at least one of the elements, optionally each of the elements, comprises a pair of gelled droplets, wherein each gelled droplet in the pair comprises said droplet medium, wherein the pair of gelled droplets is not in contact with any other gelled droplet in the droplet array, and wherein the two gelled droplets in the pair are adhered to one another by the gelled natural extracellular matrix material.

Typically, one gelled droplet in the or each pair of gelled droplets contains a first type of biological cell and the other gelled droplet in the or each pair of gelled droplets contains a second type of biological cell which is different from the first type. The first type of biological cell may be any biological cell as described herein. The second type of biological cell may be any biological cell as described herein. Typically, the first type and the second type of biological cell are mammalian cells, preferably human cells. For instance, the first type and the second type of biological cell may be selected from human liver cells, ovarian cells, breast cells or brain cells. The biological cells may be different types of cancer cells. Typically the biological cells are selected from cell types found in soft tissue. For instance, the first type of biological cell and the second type of biological cell are selected from brain cells, typically human brain cells. The first type of biological cell and the second type of biological cell may be brain cells selected from neural stem cells, neural progenitor cells, neurons, astrocytes, microglia and endothelial cells.

Both of the gelled droplets in the or each pair may contact the substrate. Alternatively, only a first gelled droplet in the or each pair may contact the substrate and the other gelled droplet in the or each pair may be disposed on top of the first gelled droplet and does not contact the substrate.

At least one of the elements, optionally each of the elements, may comprises a group of at least three gelled droplets, wherein each of the at least three gelled droplets comprises said droplet medium. At least one of the elements, optionally each of the elements, may comprises a group of at least three gelled droplets, wherein each of the at least three gelled droplets comprises said droplet medium, wherein the group of at least three gelled droplets is not in contact with any other gelled droplet in the droplet array, and wherein each of the at least three gelled droplets in the group is adhered to another of the at least three gelled droplets in the group by the gelled natural extracellular matrix material.

One gelled droplet in the or each group of at least three gelled droplets may contain a first type of biological cell and another gelled droplet in the or each group of at least three gelled droplets may contain a second type of biological cell which is different from the first type. The first type of biological cell may be any biological cell as described herein. The second type of biological cell may be any biological cell as described herein. Typically the biological cells are selected from cell types found in soft tissue. Typically, the first type of biological cell and the second type of biological cell are selected from brain cells, typically human brain cells. The first type of biological cell and the second type of biological cell may be brain cells selected from neural stem cells, neural progenitor cells, neurons, astrocytes, microglia and endothelial cells. One gelled droplet in the or each group of at least three gelled droplets may contain a first type of biological cell and another gelled droplet in the or each group of at least three gelled droplets may contain a second type of biological cell which is different from the first type and another gelled droplet in the or each group of at least three gelled droplets may contain a third type of biological cell which is different from the first and second type. The first type of biological cell may be any biological cell as described herein. The second type of biological cell may be any biological cell as described herein. The third type of biological cell may be any biological cell as described herein. The first, second and third types of biological cell may each be selected from any biological cells, for instance cells originating from any plant, animal, fungus or single-celled organism. Typically, the first, second and third types of biological cell are each selected from mammalian cells, preferably human cells. For instance, the first, second and third types of biological cell may each be selected from human liver cells, ovarian cells, breast cells or brain cells. The first, second and third types of biological cell may each be selected from cancer cells. Typically the biological cells are selected from cell types found in soft tissue, for instance brain cells. Thus, the first, second and third types of biological cell may each be selected from brain cells, typically human brain cells, for instance from neural stem cells, neural progenitor cells, neurons, astrocytes, microglia or endothelial cells. Preferably, at least one of the first, second and third types of biological cell are neural stem cells.

The at least three gelled droplets in the or each group may be arranged in a micropattern. For instance, the at least three gelled droplets in the or each group may be arranged to form a row of the least three gelled droplets along the substrate or to form a tower of the least three gelled droplets protruding up from the substrate.

Typically, each of said gelled droplets has a diameter of equal to or less than 1 mm; optionally equal to or less than 250 μm, equal to or less than 150 μm, equal to or less than 90 μm, or equal to or less than 40 μm; preferably wherein each of said droplets has a diameter of from 10 μm to 250 μm. Each of said gelled droplets may have a volume of equal to or less than 2 μL; preferably wherein each of said gelled droplets has a volume of equal to or less than 100 nL, equal to or less than 350 pL, equal to or less than 150 pL, or equal to or less than 45 pL; for instance from 0.001 nL (1 pL) to 100 nL, or from 0.001 nL (1 pL) to 500 pL.

The apparatus and printing processes described herein allow a high degree of control over the droplet size. By controlling the concentration of the cells in the droplet medium, the number of cells in the droplets produced can be controlled. Thus, the present invention makes it possible to produce droplet assemblies wherein each droplet contains a small number of cells. Typically, at least 20% of the gelled droplets in the array, preferably at least 30% of the gelled droplets in the array, more preferably at least 40% of the gelled droplets in the array, optionally at least 70% of gelled droplets in the array, for instance all of the gelled droplets in the array, each comprise a specific number, c, of biological cells. Typically, c is an integer of from 1 to 20. Preferably c is from 1 to 10, for instance c may be from 1 to 5, for example c may be 1.

Typically the biological cells are selected from cell types found in soft tissue. Typically, the one or more biological cells are selected from brain cells, typically human brain cells, optionally wherein the brain cells are selected from neural stem cells, neural progenitor cells, neurons, astrocytes, microglia and endothelial cells. Preferably, the brain cells are neural stem cells or astrocytes. At least 5% of the gelled droplets in the array, preferably at least 10% of the gelled droplets in the array, more preferably at least 15% of the gelled droplets in the array, optionally at least 50% of the gelled droplets in the array, for instance all of the gelled droplets in the array, may each comprise a first type of biological cell and a second type of biological cell which is different from the first type of biological cell. The first type of biological cell may be any biological cell as described herein. The second type of biological cell may be any biological cell as described herein. Typically, the first type of biological cell and the second type of biological cell are selected from brain cells, typically human brain cells, optionally wherein the first type of biological cell and the second type of biological cell are brain cells selected from neural stem cells, neural progenitor cells, neurons, astrocytes, microglia and endothelial cells.

The gelled natural extracellular matrix material may be obtainable by gelling any natural extracellular matrix material as described herein. Typically, the gelled natural extracellular matrix material and the biological cells together form at least 80% by volume of the droplet medium, preferably at least 90% by volume of the droplet medium, more preferably at least 95% by volume, for instance at least 99% by volume, of the droplet medium. Preferably, the droplet medium comprises an undiluted gelled natural extracellular matrix material and one or more biological cells. Preferably, the droplet medium comprises undiluted gelled matrigel and one or more biological cells. The droplet medium may consist of a gelled natural extracellular matrix material and one or more biological cells. Preferably, the gelled natural extracellular matrix material is gelled matrigel. For instance, the droplet medium may consist of gelled matrigel and one or more biological cells.

The gelled natural extracellular matrix material may comprise collagen.

Typically, the number of elements in the array is greater than 10, preferably greater than 100. The number of gelled droplets in the array is greater than 10, preferably greater than 100, optionally greater than 1000, for example greater than 10000. In this case the number of gelled droplets includes all gelled droplets making up each individual element in the array, in the situation where each element comprises multiple droplets.

The substrate may be any suitable substrate. The substrate typically comprises a polymer or glass. Typically, the substrate comprises a polymer, such as poly(methyl methacrylate) or polystyrene.

The substrate typically is a polymer or glass. Typically, the substrate is a polymer, such as poly(methyl methacrylate) or polystyrene. The substrate may comprise a plurality of microwells. Each element in the array may be disposed in a microwell on the substrate.

Typically, the droplet array is disposed in an aqueous medium. Preferably, the aqueous medium is cell culture medium.

Alternatively, the droplet array may be disposed in a bulk medium, which may be any bulk medium as described herein. Typically, the bulk medium is a hydrophobic medium.

Nano-Bioreactors

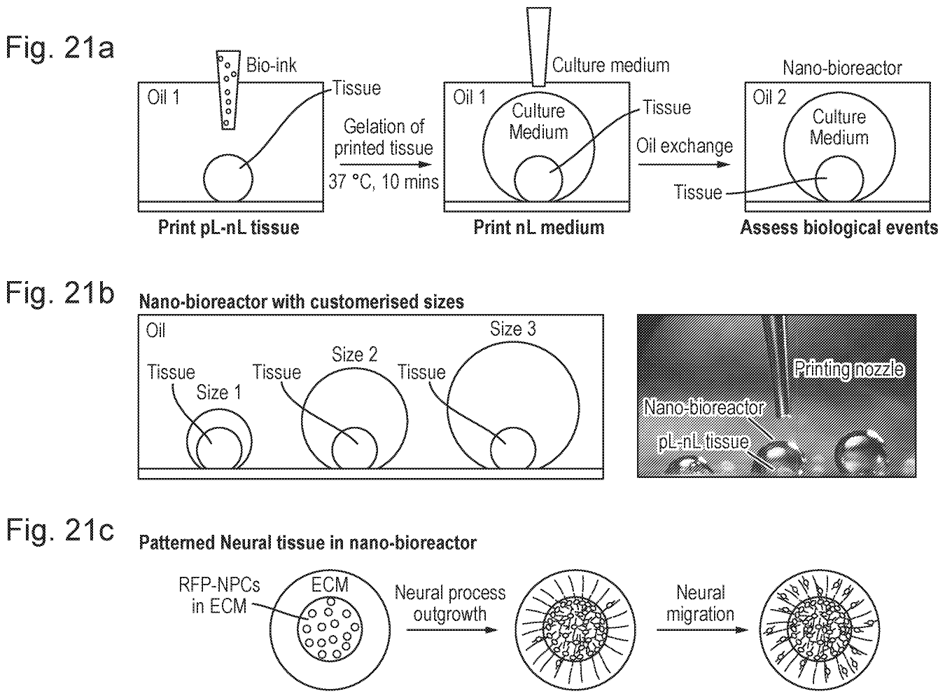

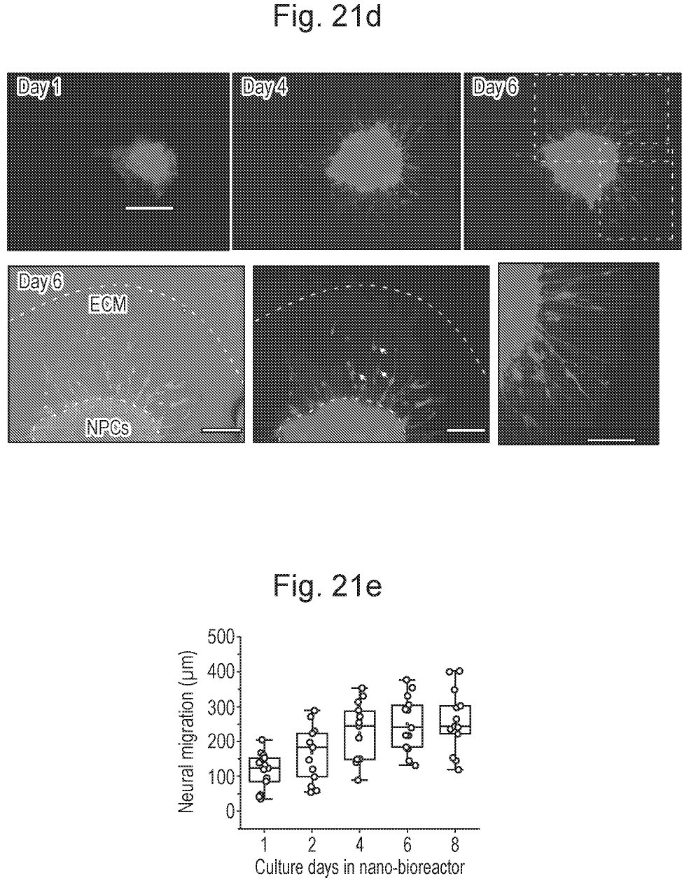

At least one element in the droplet array may comprise a droplet of a culture medium. Thus, at least one of the elements, optionally each of the elements, may comprise a droplet of a culture medium. Typically, within each element in the array, the droplet of culture medium surrounds the droplets comprising cells. Such a structure may be referred to as a “nano-bioreactor” and an example is provided in FIG. 21. The culture medium may be any culture medium suitable for culturing the cell types present within the element. For instance the culture medium may be an extra cellular matrix material as described herein. The culture medium may be Matrigel. Typically, the culture medium is printed onto the array using the techniques described herein.

Thus, the invention also provides a nano-bioreactor comprising at least one droplet which comprises a droplet medium which comprises one or more biological cells and a natural extracellular matrix material, and at least one droplet of culture medium.

The biological cells may be any biological cells as described herein. The natural extracellular matrix material may be any natural extracellular matrix material as described herein. The culture medium may be any culture medium suitable for culturing the cell types present within the element. For instance the culture medium may be an extra cellular matrix material as described herein. The culture medium may be Matrigel. Typically the droplet of culture medium surrounds the droplet or droplets comprising droplet medium.

Often, the nano-bioreactor comprises amphipathic molecules. The amphipathic molecules may be any amphipathic molecules as described herein. Thus, the nano-bioreactor may comprise at least one droplet which comprises (i) a droplet medium which comprises one or more biological cells and a natural extracellular matrix material and (ii) an outer layer of amphipathic molecules. Alternatively, it may be that the nano-bioreactor comprises one or more droplets which do not comprise amphipathic molecules; those one or more droplets may consist only of the droplet medium.

The invention also provides an array of nano-bioreactors comprising a plurality of nano-bioreactors spaced apart from one another on a substrate in a bulk medium, wherein each nano-bioreactor comprises at least one droplet which comprises a droplet medium which comprises one or more biological cells and a natural extracellular matrix material, and at least one droplet of culture medium, wherein the bulk medium and the droplet medium are immiscible. Typically, the bulk medium and the culture medium are also immiscible.

The natural extracellular matrix material may be any natural extracellular matrix material as described herein. The substrate may be any substrate as described herein. The culture medium may be any culture medium suitable for culturing the cell types present within the nano-bioreactor. For instance the culture medium may be an extra cellular matrix material as described herein. The culture medium may be Matrigel.

The bulk medium may be any bulk medium as described herein. Typically the bulk medium is one which is compatible, i.e. non-toxic, to the cells of interest. Preferably, the bulk medium is a hydrophobic medium which is non-toxic to the cells within the array. One example of such a bulk medium is a naturally occurring oil, for instance sunflower oil. Thus the bulk medium may comprise or consist of sunflower oil, or a constituent thereof. For instance, the bulk medium may comprise or consist glyceryl trioleate and/or glyceryl trilinoleate.

The invention provides a nano-bioreactor comprising at least one gelled droplet, wherein each gelled droplet comprises a droplet medium which comprises one or more biological cells and a gelled natural extracellular matrix material, and at least one droplet of culture medium. In some instances, the culture medium is also be gelled.

The biological cells may be any biological cells as described herein. The natural extracellular matrix material may be any natural extracellular matrix material as described herein. The culture medium may be any culture medium suitable for culturing the cell types present within the element. For instance the culture medium may be an extra cellular matrix material as described herein. The culture medium may be Matrigel. Typically the droplet of culture medium surrounds the droplet or droplets comprising droplet medium.

The invention also provides an array of nano-bioreactors comprising a plurality of nano-bioreactors spaced apart from one another on a substrate in a bulk medium, wherein each nano-bioreactor comprises at least one gelled droplet which comprises a droplet medium which comprises one or more biological cells and a gelled natural extracellular matrix material, and at least one droplet of culture medium, wherein the bulk medium and the droplet medium are immiscible. Typically, the bulk medium and the culture medium are immiscible.

The natural extracellular matrix material may be any natural extracellular matrix material as described herein. The substrate may be any substrate as described herein. The culture medium may be any culture medium suitable for culturing the cell types present within the element. For instance the culture medium may be an extra cellular matrix material as described herein. The culture medium may be Matrigel.

The bulk medium may be any bulk medium as described herein. Typically the bulk medium is one which is compatible, i.e. non-toxic, to the cells of interest. Preferably, the bulk medium is a hydrophobic medium which is non-toxic to the cells within the array. One example of such a bulk medium is a naturally occurring oil, for instance sunflower oil. Thus the bulk medium may comprise or consist of sunflower oil, or a constituent thereof. For instance, the bulk medium may comprise or consist glyceryl trioleate and/or glyceryl trilinoleate.

The nano-bioreactors as described herein may comprise two or more droplets or three or more droplets. Typically, the nano-bioreactor comprises a pair of droplets, wherein each gelled droplet in the pair comprises said droplet medium.

The nano-bioreactors as described herein may comprise a mixture of biological cell types. For instance, the nano-bioreactors as described herein may comprise a first droplet comprising a droplet medium which comprises one or more of a first type of biological cell and a natural extracellular matrix material and a second droplet comprising a droplet medium which comprises one or more of a second type of biological cell and a natural extracellular matrix material. Typically, the second type of biological cell which is different from the first type. The first type of biological cell may be any biological cell as described herein. The second type of biological cell may be any biological cell as described herein. Typically, the first type and the second type of biological cell are mammalian cells, preferably human cells. For instance, the first type and the second type of biological cell may be selected from human liver cells, ovarian cells, breast cells or brain cells. The biological cells may be different types of cancer cells. Typically the biological cells are selected from cell types found in soft tissue. For instance, the first type of biological cell and the second type of biological cell are selected from brain cells, typically human brain cells. The first type of biological cell and the second type of biological cell may be brain cells selected from neural stem cells, neural progenitor cells, neurons, astrocytes, microglia and endothelial cells. For instance the first type of biological cell may be neuroprogenitor cells and the second type of biological cell may be astrocytes.

Use of Droplet Array in High Throughput Screening

The invention also provides the use of a droplet array of the invention in high throughput screening.

The invention also provides a method of screening a test substance which comprises providing a droplet array of the invention, contacting the test substance with at least one of the elements of the array, and measuring a response.

Preferably, the droplet array of the invention which is used, or which is provided in the method of screening, is a droplet array which comprises a plurality of elements spaced apart from one another on a substrate, wherein each element comprises at least one gelled droplet, wherein each gelled droplet comprises a droplet medium which comprises one or more biological cells and a gelled natural extracellular matrix material. The droplet array of the invention which is used, or which is provided in the method of screening, may be as further defined anywhere herein for the droplet array of the invention, for instance it may be one which is disposed in an aqueous medium, for instance in cell culture medium.

The method may be for screening a plurality of test substances, which method comprises contacting each of the test substances with one or more elements of the array, and measuring a response for each of the test substances. For instance, contacting each of the test substances with one or more elements of the array may comprise printing a droplet comprising a droplet medium comprising the test substance onto each element in the array, such that the droplet comprising a droplet medium comprising the test substance and the element are contacting. Thus, the arrays of the present invention may be used to test multiple substances at once using small volumes of test substance. Thus the methods of screening of the invention may be less resource-intensive that known screening methods, and may require lower volumes of test substance. Typically, the test substance is a drug. Thus, the droplet array of the invention which is used, or which is provided in the method of screening, may be used in drug testing. The droplet array of the invention which is used, or which is provided in the method of screening may be used in a neurotoxicity test.

Process for Producing a Droplet Array

The invention also provides a process for producing a droplet array, which droplet array comprises a plurality of elements spaced apart from one another on a substrate in a bulk medium, wherein each element comprises at least one droplet which comprises a droplet medium which comprises one or more biological cells and a natural extracellular matrix material, wherein the bulk medium and the droplet medium are immiscible; which process comprises generating a plurality of droplets in the bulk medium, wherein each of said droplets comprises a droplet medium which comprises one or more biological cells and a natural extracellular matrix material, and arranging the droplets on the substrate in the bulk medium to form said plurality of elements spaced apart from one another, wherein each element comprises at least one of said droplets.

In the process, each element may comprise at least one droplet which comprises: (i) a droplet medium which comprises one or more biological cells and a natural extracellular matrix material, and (ii) an outer layer of amphipathic molecules around the surface of the droplet medium, wherein the bulk medium and the droplet medium are immiscible; which process comprises generating a plurality of droplets in the bulk medium, wherein each of said droplets comprises: (i) a droplet medium which comprises one or more biological cells and a natural extracellular matrix material, and (ii) an outer layer of amphipathic molecules around the surface of the droplet medium, and arranging the droplets on the substrate in the bulk medium to form said plurality of elements spaced apart from one another, wherein each element comprises at least one of said droplets.

The droplet array may be any droplet array as described herein.

Typically, the steps of generating the droplets in the bulk medium and contacting each of the droplets with another of the droplets are performed at a droplet printing temperature. The droplet printing temperature may be any droplet printing temperature as described herein. Typically, the droplet printing temperature is a temperature below a temperature at which the droplet medium is capable of forming a gel. Usually, the steps of generating the droplets in the bulk medium and arranging the droplets on the substrate in the bulk medium are performed while the temperature of the bulk medium is maintained at a droplet printing temperature. The droplet printing temperature may be a temperature of less than 10° C., preferably a temperature of from 2° C. to 8° C., for instance at a temperature of about 5° C.

Typically, the process further comprises gelling the natural extracellular matrix material in the droplet array. Typically, gelling is induced by a change in temperature, for instance heating or cooling the droplet medium from the temperature at the time of generating a droplet. Preferably, the droplet medium is capable of gelation on heating. Thus, typically, gelling the natural extracellular matrix material comprises increasing the temperature of the bulk medium. For instance, gelling the natural extracellular matrix material may comprise increasing the temperature of the bulk medium to a first temperature which is above said droplet printing temperature. Typically, the temperature of the bulk medium is increased to said first temperature, which is a temperature of from 10° C. to 30° C., preferably from 20° C. to 30° C., for instance about 25° C. The temperature of the bulk medium may be held at the first temperature for at least 10 minutes.

The process may further comprise a step of increasing the temperature of the bulk medium to a second temperature which is higher than the first temperature, preferably wherein the second temperature is a temperature of from 30° C. to 40° C., preferably a temperature of from 35° C. to 40° C., for instance about 37° C. The temperature of the bulk medium may be held at the second temperature for at least 30 minutes, preferably at least 1 hour.

The process may comprise recovering the droplet array from the bulk medium. Typically, recovering comprises recovering the substrate, which substrate has the plurality of elements spaced apart from one another disposed thereon, from the bulk medium. Recovering the droplet array from the bulk medium may comprises substituting an aqueous medium for the bulk medium, preferably wherein the aqueous medium is cell culture medium. Typically, recovering the droplet array from the bulk medium comprises (i) replacing at least part of the bulk medium with fresh bulk medium in order to reduce the concentration of amphipathic molecules (if present) in the bulk medium, and subsequently (ii) replacing the bulk medium with an aqueous medium, preferably wherein the aqueous medium is cell culture medium.

If the intention is to continue to culture the cells in the array within the bulk medium, the bulk medium may be substituted for a second bulk medium which is compatible, i.e. non-toxic, to the cells of interest. Therefore, in some instances the elements may be printed within a first bulk medium as described herein, which is substituted for a second bulk medium which is compatible with the cells. One example of such a bulk medium is a naturally occurring oil, for instance sunflower oil. Thus the bulk medium may comprise or consist of sunflower oil, or a constituent thereof. For instance, the bulk medium may comprise or consist glyceryl trioleate and/or glyceryl trilinoleate.

The invention also provides a droplet array which is obtainable by the process of the invention for producing a droplet array.

These processes may be adapted to produce a nano-bioreactor or an array of nano-bioreactors as described herein. Thus, the invention also provides a process for producing a nano-bioreactor on a substrate in a bulk medium, wherein the nano-bioreactor comprises at least one droplet which comprises a droplet medium which comprises one or more biological cells and a natural extracellular matrix material and at least one droplet of culture medium, wherein the bulk medium and the droplet medium are immiscible; which process comprises generating at least one droplet in the bulk medium, wherein the droplet comprises a droplet medium which comprises one or more biological cells and a natural extracellular matrix material, and arranging the droplet on the substrate in the bulk medium; and which process comprises generating at least one droplet in the bulk medium, wherein the droplet comprises culture medium, and arranging the droplet comprising culture medium so that it is contacting at least one droplet comprising droplet medium, to form said nano-bioreactor. The process may further comprise gelling the natural extracellular matrix material in the nano-bioreactor. The gelling may be induced by any method as described herein. The process may be repeated multiple times to generate an array of nano-bioreactors spaced apart on a substrate.

### EXAMPLES

### Example 1—Pre-Patterned Tissues

Experimental Section

Printer setup. The printer was modified from that previously described,^{[19]} to adapt it to viscous materials and high cell densities (up to 3.5×10⁷ mL⁻¹). A more powerful piezo-electric driver with voltage limits of ±130 V was used to produce a higher ejection force (further details below). A Peltier-based temperature-controlled stage was attached to the printer with a temperature range of −15 to 80° C. For all experiments, Matrigel-based bio-ink was printed at ˜5° C. Glass printing nozzles were modified with (3-aminopropyl)trimethoxysilane (Sigma Aldrich, 281778) to provide a hydrophilic coating, which prevented printing oil from entering the nozzle. The printing oil comprised 4 mg mL⁻¹ DPhPC (1, 2-diphytanoyl-sn-glycero-3-phosphocholine, Avanti, 850356) in a mixture of undecane and silicone oil AR20 (both from Sigma Aldrich; v:v, 1:4).

Cell culture and harvest. Two sources of iPSC-derived hNSCs were used: ax0018 from Axol and hNSCs kindly provided by Dr Sally Cowley (James Martin Stem Cell Facility, Oxford). The ax0018 cells were derived from human fibroblasts (healthy donor) and express typical markers of cerebral cortical neural stem and progenitor cells, such as PAX6, FOXG1 and nestin (manufacturer's data). The cells were cultured according to the manufacturer's instructions. Briefly: the cells were thawed and seeded on 2% Matrigel (Corning, 354230)-coated 6-well plates in Neural Plating-XF medium (Axol, ax0033), followed by expansion on the second day with Neural Expansion-XF medium (Axol, ax0030), containing EGF (20 ng mL⁻¹, Axol, ax0047X) and FGF2 (20 ng mL⁻¹, Axol, ax0047). Cells were harvested at ˜80% confluency (ranging from 4-7 days post-plating) for bio-ink preparation. The NSCs provided by Dr Cowley were human fibroblasts reprogrammed according to the protocol of Shi et al.[2] The resulting cortical neural stem cells were grown as 2D adherent cultures on Matrigel-coated 6-well plates in neural maintenance medium (NMM): 1:1 (v/v) N2 medium and B-27 medium. N-2 medium contains advanced DMEM/F12 (Gibco, 12634-010), 1×N-2 (Gibco, 17502048), 5 μg mL⁻¹ insulin (Sigma, I9278), 100 μM 2-mercaptoethanol (Gibco, 31350-010), 1 mM GlutaMax (Gibco, 35050-038), 100 M non-essential amino acids (NEAA) (Gibco, 1605380), and 50 U mL⁻¹ penicillin and streptomycin (Gibco, 15140-122). B-27 medium contains neurobasal medium (Gibco, 21103-049S), 1×B-27 (Gibco, 17504044), 1 mM GlutaMax (Gibco, 35050-038), and 50 U mL⁻¹ penicillin and streptomycin (Gibco, 15140-122). Cultured hiPSC-NSCs were harvested between day 30 and 45 of the differentiation protocol. Cells were incubated in accutase solution (Sigma Aldrich) for 5 min at 37° C. and dissociated to a single cell solution by gentle pipetting. We did not observe obvious differences after printing between the two iPSC-NSC lines.

RFP-hNSCs derived from RFP-iPSCs were also provided by Dr Sally Cowley.^{[35]} The cells were cultured and passaged the same as the non-labelled hNSCs from Dr Sally Cowley except the addition of 2.5 μg/mL puromycin in NMM for RFP selection.

iPSC-derived human cortical neurons (hCNs) were purchased from Elpis Biomed Ltd. and thawed according to the manufacturer's instructions. The cells were used directly for bio-ink after thawing, without culture.

Human primary astrocytes (hAs, ScienCell™, catalogue #1800) were cultured according to the manufacturer's instructions. Briefly: the cells were thawed and cultured on poly-L-lysine (Sigma, P4707) coated flasks in astrocyte medium containing: DMEM/F12 (Gibco, 11320033), 1×N-2 (Gibco, 17502048), 1×B-27 (Gibco, 17504044), 100 μM NEAA (Gibco, 1605380), 100 μM 2-mercaptoethanol (Gibco, 31350-010), 10 ng mL⁻¹ FGF2 (Gibco, 13256029), 10% FBS (Gibco, 10270-106), and 1 mM GlutaMax (Gibco, 35050-038). Half of the culture medium was exchanged with fresh medium every second day. Cells were harvested by incubation with 1×trypsin-EDTA solution (0.5 g L⁻¹ trypsin, Sigma Aldrich, T3924) for 3 min according to the manufacturer's instructions.

Bio-ink preparation and printing procedure. Prior to use, total cell number and cell viability were determined by trypan blue staining by using a Countess II automated cell counter (ThermoFisher). Matrigel was used without dilution and thawed at 4° C. before use. Cells were dissociated to form suspensions of individual cells. After centrifugation (5 min at 200×g), the supernatant was removed. A volume of thawed Matrigel, calculated based on the required cell-density of the bio-ink, was then added to the pellet and the cells were suspended on ice to generate the bio-ink, which was loaded into printer nozzles and kept at ˜4° C. during the printing process. Printing was performed by using maps entered into the custom control software as previously described.^{[19]} The printing process took 1-2 h to generate multiple droplet networks.

Phase transfer of printed network. Printed networks were incubated at room temperature for 30 min before transfer to a tissue culture incubator at 37° C. for 2 h. Half of the oil was then removed and replaced with silicone oil. This oil exchange process was repeated five times to dilute lipid. Culture medium was then added to replace half of the oil. The medium was then exchanged a further four times (half of the medium was exchanged each time) to remove any residual oil.

The phase-transferred tissue constructs with hNSCs (with and without astrocytes) were then cultured in NMM to support differentiation. Two thirds of the medium was changed every 2-3 days.

Printed tissues with hCNs (with and without astrocytes) were cultured in comp:GCN medium with 10 μM ROCK inhibitor (Stratech Scientific S1049-SEL) and 1 μg mL⁻¹ doxycycline (Sigma Aldrich, D9891) for the first 2 days and doxycycline only for another 2 days. Comp:GCN medium alone was then used for up to 10 days. Half of the medium was exchanged with fresh medium every second day. The comp:GCN medium contains neurobasal medium (Gibco, 21103-049S), 1 mM GlutaMax (Gibco, 35050-038), 25 μM 2-mercaptoethanol (Gibco, 31350-010), 1×B-27 (Gibco, 17504044), 10 ng mL⁻¹ NT3 (R & D, 267-N3-025), 5 ng mL⁻¹ BDNF (R & D, 248-BD-005).

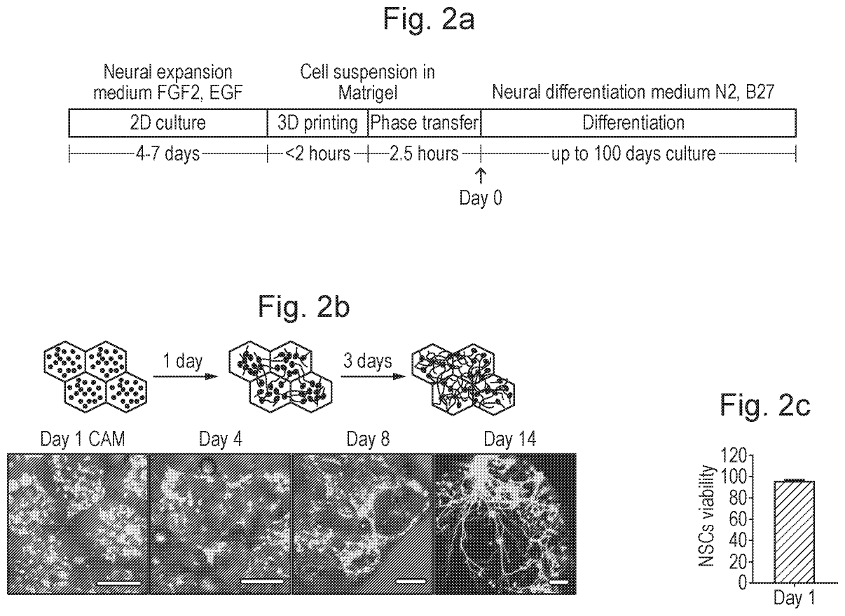

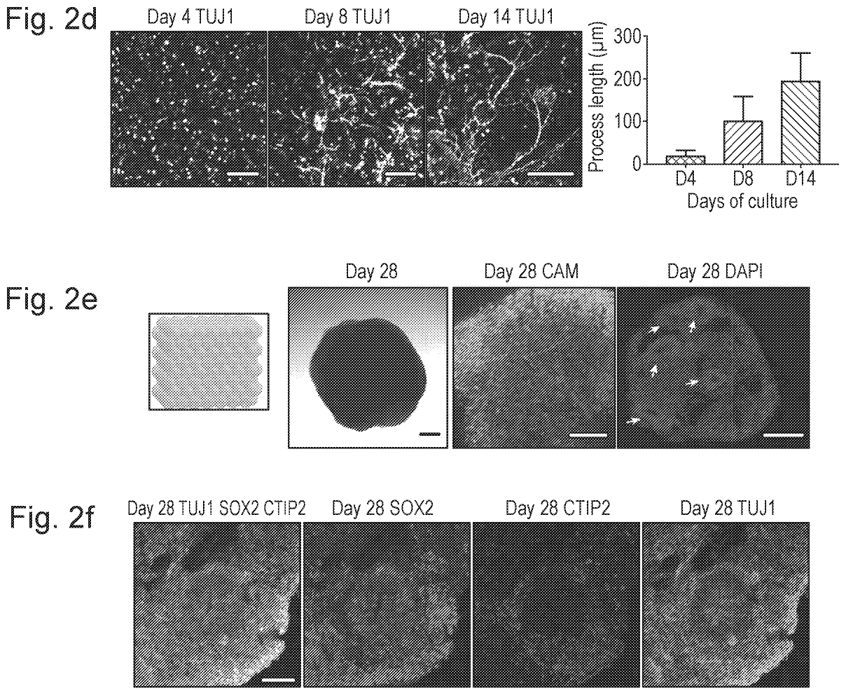

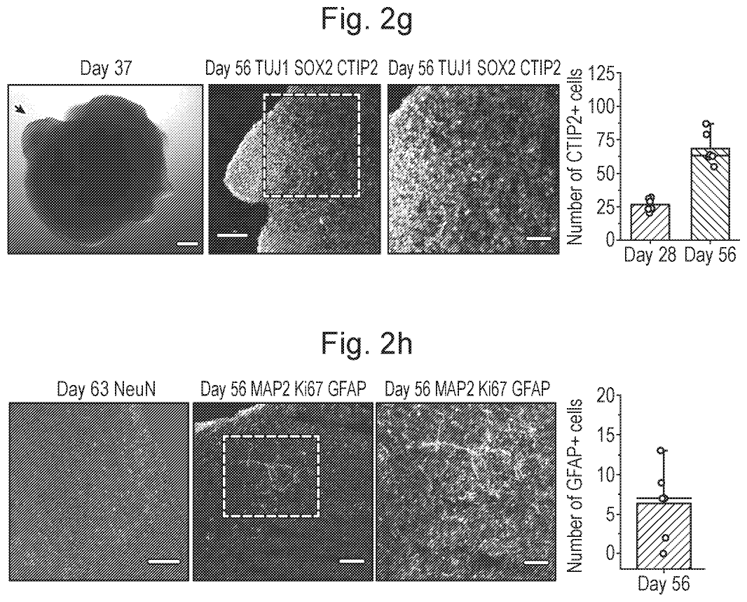

Cell staining and viability calculation. Printed tissues were incubated with 2.5 μM calcein-AM (Cambridge Biosciences Ltd) and 5.0 μM propidium iodide (Sigma Aldrich) for 30 min before imaging. Four randomly selected fields of each printed network at different Z-heights were imaged with ˜30× magnification by using fluorescence confocal microscopy (Leica SP5). Dead cells were counted manually due to their low number. Live cells were counted using Fiji1³⁶1 by setting a threshold matches the CAM fluorescence of the live cells, followed by particle analysis (size setting: 2 micron²-Infinity). The cell number of clusters of cells was determined by using the area of a cluster divided by the area of an average cell. The live/dead numbers from the four images of one sample were averaged to give each data point and five samples were used to determine the viability as shown in FIG. 2c (n=5).

Tissue sectioning, immunostaining, and imaging. Printed tissues were fixed in 4% (v/v) paraformaldehyde (Sigma Aldrich) for 30 min at room temperature and then quenched in 50 mM glycine (Sigma Aldrich). Tissue sectioning was performed using a cryostat to generate 30 μm-thick sections for immunohistological analysis. Samples were incubated at 37° C. for 15 min, washed in PBS and then blocked for 1 h at room temperature with 5% (v/v) goat serum (Sigma Aldrich) in TPBS containing 0.1% v/v Triton X-100 (Fisher Scientific). Primary antibodies (Table 1) were added in blocking solution and samples were incubated for 2 h at 37° C. Subsequently, samples were washed in PBS then incubated with secondary antibodies for 2 h at 37° C. Samples were then washed in PBS, incubated with DAPI (1/1000 dilution) in TPBS for 15 min and washed again. All printed networks were imaged using fluorescence confocal microscope (Leica SP5) and wide-field light microscope (Leica DMI 8).

Process length analysis. Tuj1 immunostaining was performed on printed tissues as described above. Imaging was conducted with a confocal microscope using a 20×objective and Z-projection images were used for quantification. The length of the TUJ1⁺ processes emanating from the cell bodies at 4, 8, 14 days ppd was quantified by using the Fiji ‘freehand line’ tool and the values are summarised in FIG. 2d (n>15, P<0.001 between all groups).

Cell number analysis of CTIP2⁺ and GFAP⁺ cells. Printed tissues were fixed and cut into 30 μm sections before immunostaining. CTIP2⁺ cells were counted as cell number per unit area in a 100 μm×100 μm field at 28 and 56 days ppd (n=6, p<0.001). GFAP⁺ cells were counted as cell number per unit area in a 200 μm×200 μm field at day 56 ppd (n=6).

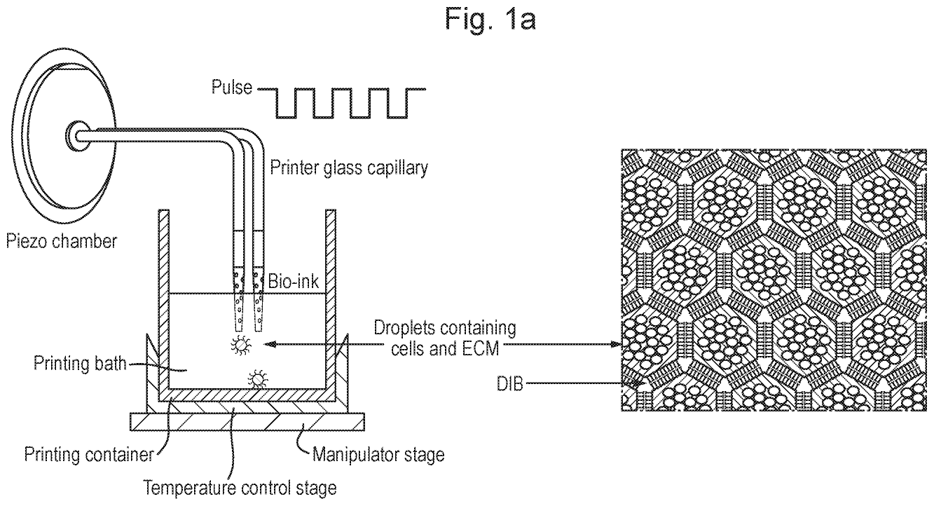

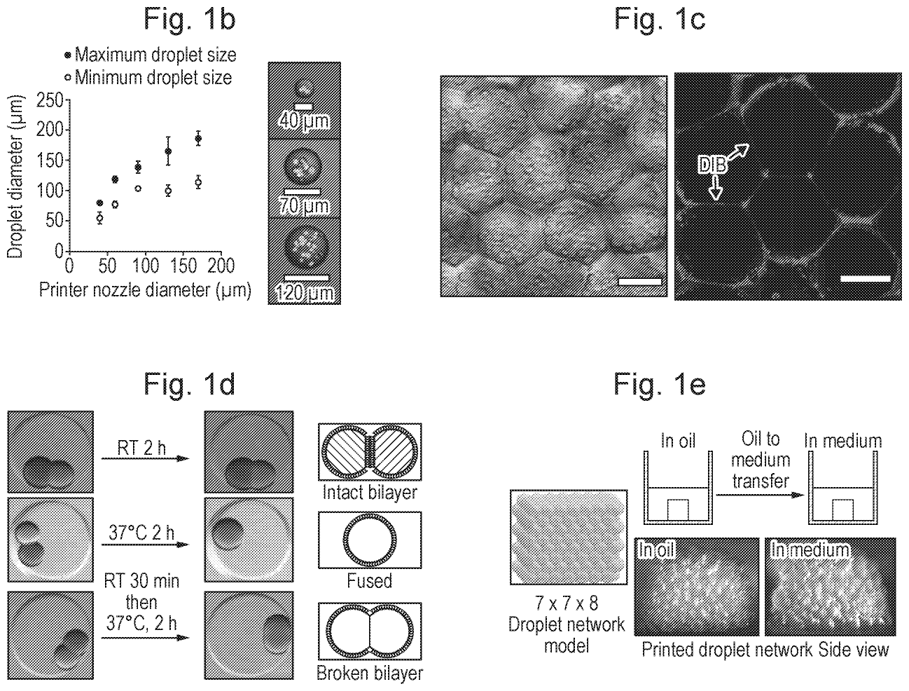

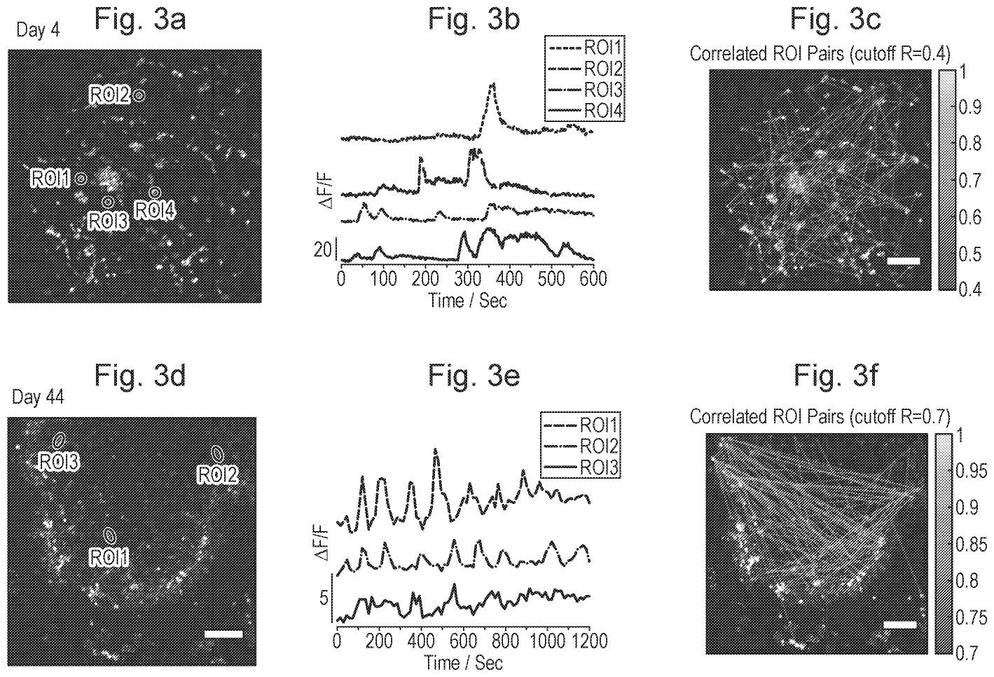

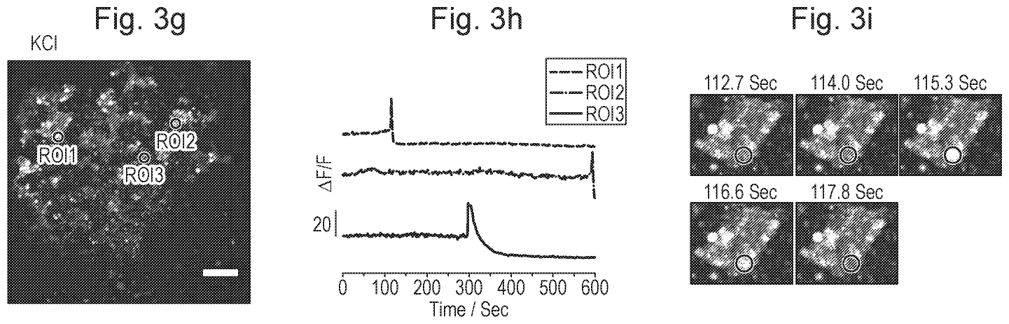

Calcium imaging and correlation analysis. A Fluo-4 Direct™ calcium assay kit (F10471, Invitrogen) was used according to the manufacturer's instructions to measure calcium activities. Briefly, printed neural tissues were transferred to 48-well plates and incubated with NMM/Fluo-4 calcium imaging reagents (v:v, 1:1) for 1 h at 37° C. Both spontaneous and evoked calcium recordings were recorded at 37° C. by fluorescence confocal microscopy (Leica SP5) at 1 frame per 2 seconds for FIG. 3a, 1 frame per 15 seconds FIG. 3d and 1 frame per 1 second for FIG. 3g. Pre-processing and analysis of time-lapse calcium recordings were performed in MATLAB. The non-rigid image registration algorithm SIFT Flow was used to correct for local deformations of printed tissue relative to the initial frame. Individual neuronal somata were manually selected as ROIs, and the pixel mask in the local area was refined using ICA-based de-mixing of fluorescent transients. ΔF/F₀ at time t was calculated with a moving baseline as:

$$\frac{F ( t )}{min ( {\overline{F ( x )}}_{( t - {τ}_{2} ) < x < t )} )}$$

$$where :$$

$$F = {F}_{raw} - {F}_{neuropil}$$

$$\overline{F ( x )} = \frac{1}{{τ}_{1}} {\int }_{x - {τ}_{1} / 2}^{x + {τ}_{1} / 2} F ( τ ) d τ$$

$${τ}_{2} = 75 seconds ( 5 frames )$$

$${τ}_{1} = 45 seconds ( 3 frames )$$

To determine the active frames of a neuron, an 8-frame moving standard deviation (amov) was calculated along the time series, with the 10^{th} percentile σₘₒᵥ taken as the deviation of baseline noise. Active frames were defined as 3·σₘₒᵥ above the mean intensity value. Cross-correlation at zero time-lag is calculated for all pairs of neurons. 99.9% confidence intervals for the correlation coefficients were determined via cross-correlation of 1000 shuffled ΔF/F₀ traces of each neuron pair, where each shuffle maintains sequential blocks of active frames (3·σₘₒᵥ) to preserve any slow transients in a calcium signal. Only pairs with coefficients >99.9 CI were considered for further analysis.

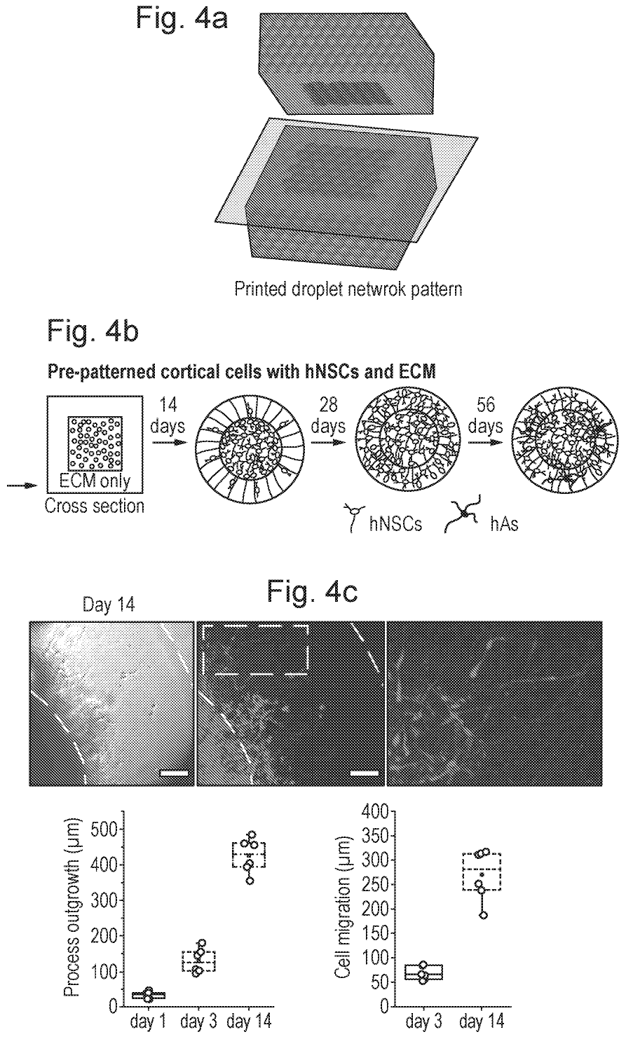

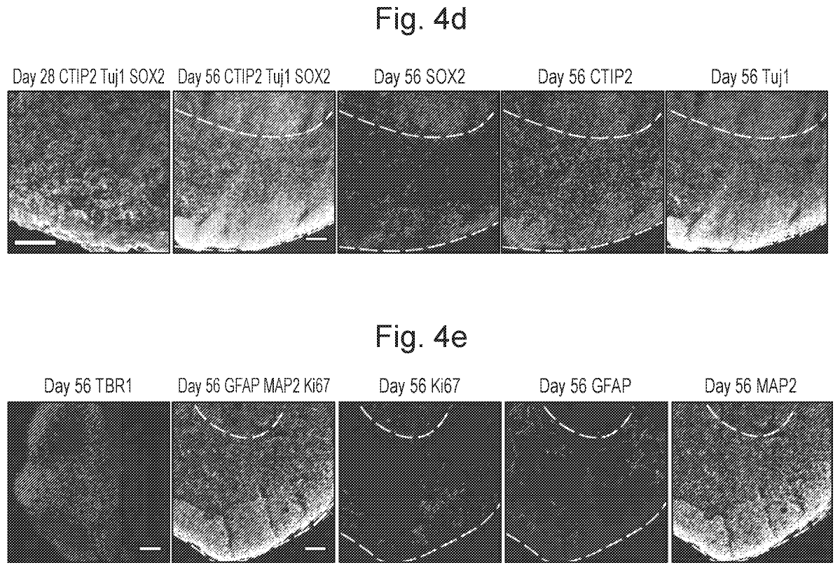

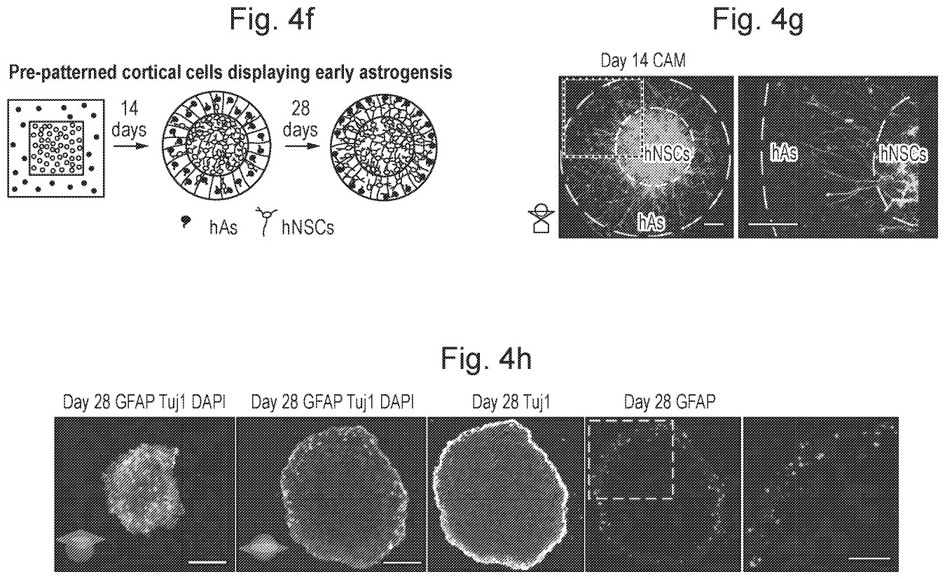

Process outgrowth and cell migration analysis. Printed tissues with RFP-hNSCs encased in Matrigel were monitored by live imaging. Both process projection and cell migration from the RFP-hNSC compartment into the Matrigel compartment were analysed using the Fiji ‘freehand tool’. The length of process outgrowth was quantified at 1, 3 and 14 days ppd (n=6 for each time points, P<0.005 between all three groups). The distance of cell migration into the cell-free Matrigel was quantified at 3 and 14 days ppd (n=6 for each time points, P<0.005). The box charts in FIG. 4c show mean, interquartile range (box) and 5th and 95th percentiles (whiskers).

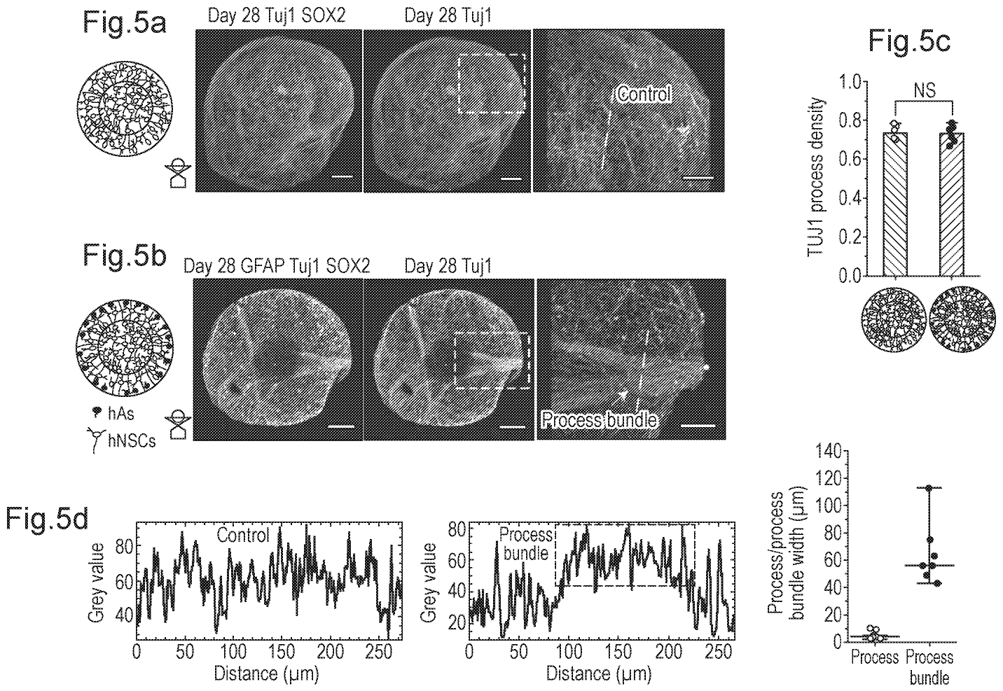

Process density, process bundle width and cell alignment analysis. Printed tissues were fixed and immunostained at 28 days ppd. Process densities were calculated as the ratios of the TUJ1*-fluorescence area to the total area (n=6, P>0.9, not significant). The fluorescence intensities of process bundles were plotted with Fiji ‘profile plots’ and the widths of the bundles were analysed in FIGS. 5d and 5f (n>7, P<0.001, for both plots). The alignment of migrating cells was quantified as the angle between the cells and associated process bundles by using the Fiji ‘angle tool’ (n=68 cells). The percentages of cells with specific ranges of angles (0°-10°, 10°-20°, . . . , 80°-90°) were calculated and presented as relative frequency in FIG. 5g.

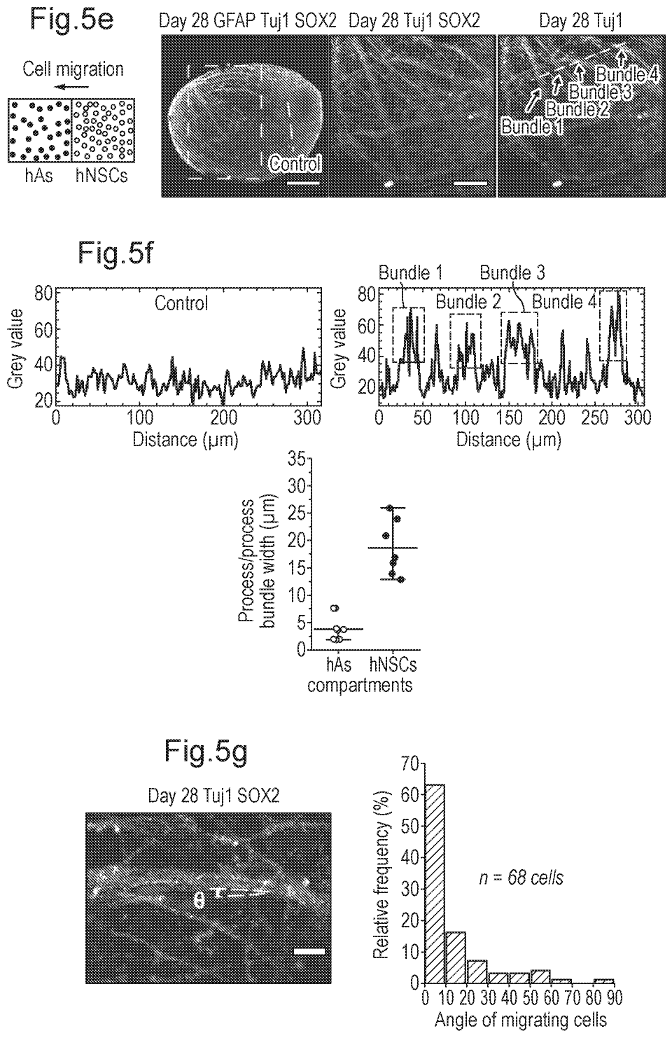

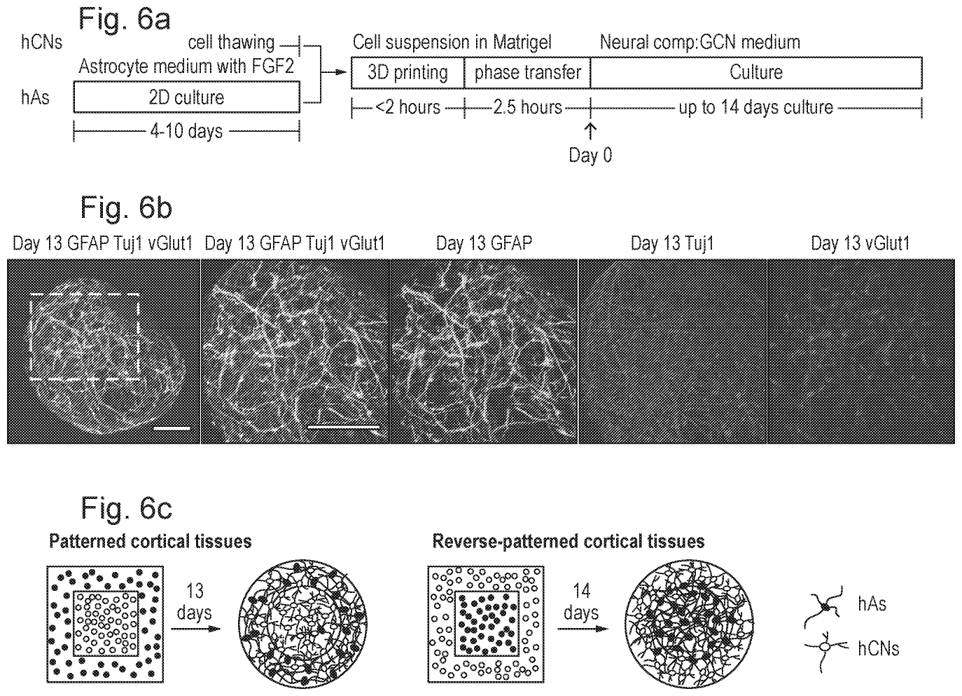

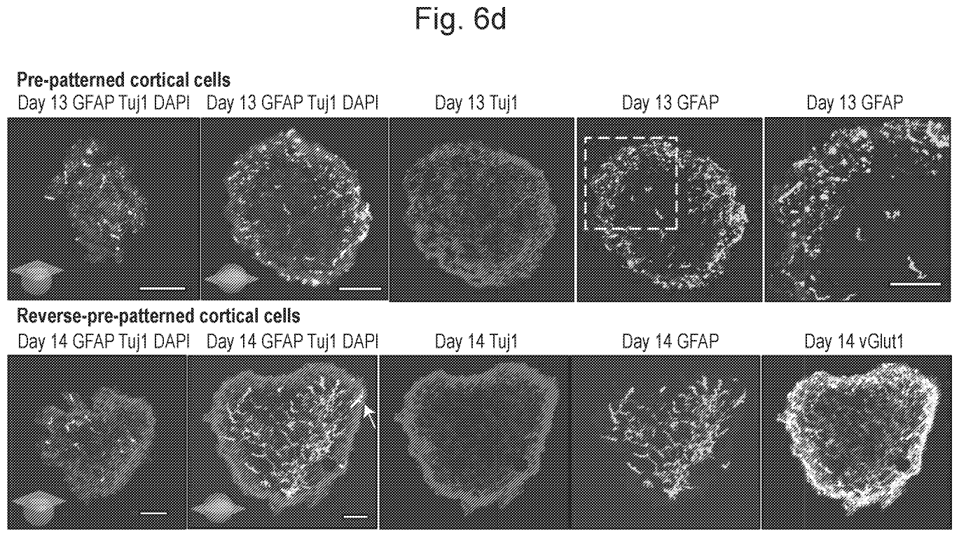

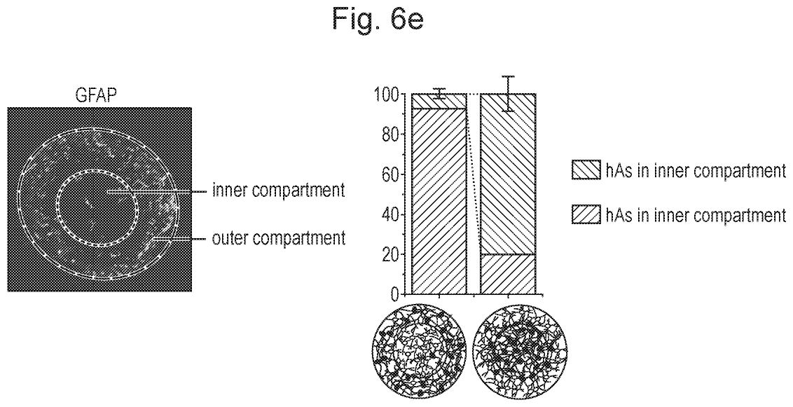

hAs segregation analysis. Printed tissues with either hCNs (in)-hAs (out) or hAs (in)-hCNs (out) patterns were sectioned, and sections from a central plane of the tissues were immunostained with antibodies for TUJ1, SOX2 and GFAP as described above. The inner and outer compartments (of equal areas) were defined as shown in FIG. 6e. Fluorescent areas (GFAP⁺) in both inner and outer compartments were quantified using Fiji to generate the stacked bar chat. n=3 for both patterns.

Statistics. Data are presented either as mean±standard deviation (FIGS. 2c, 2d and 6c) or mean with 5th and 95th percentiles and/or interquartile range (FIG. 2g, 2h, 4c, 5c, 5d, and 5f). Statistical analyses were performed using GraphPad Prism 8 or Origin. Statistical analysis was performed using the t-test for two groups or one way ANOVA with Tukey's multiple comparisons test when more than two groups were compared.

Printing oil. Printing oil comprised a mixture of undecane and silicone oil AR20 (1:4, v:v, Sigma Aldrich) as opposed to the hexadecane and silicone oil (1:1, v:v) mixture used previously (Villar, G., Graham, A. D. & Bayley, H. A tissue-like printed material. Science 340, 48-52, doi: 10.1126/science.1229495 (2013)). This is due to the fact that hexadecane has a melting point of 18° C. and therefore freezes at the printing temperature (˜ 5° C.) required for Matrigel. Notably, different ratios of undecane and silicone oil (from 1:1 to 1:8, v:v) supported DIB formation. The higher proportion of silicone oil used allowed us to match the increased density of droplets that contain ECM and a high density of cells.

Printing different matrices. Matrigel and collagen (Corning, 354236) were printed at 5-8° C. and chitosan (Sigma Aldrich) was printed at room temperature. Chitosan: 2.5% (w/v); Matrigel: undiluted, protein concentration 8.2 mg mL⁻¹; collagen: 3.6 mg mL⁻¹, neutralised prior to print. In general, higher voltage was required to eject droplets containing materials with higher viscosity.

Neural process outgrowth analysis. Printed neural tissues were cultured in NMM. 2.5 μM calcein-AM (Cambridge Biosciences Ltd) was added 30 min before imaging the tissues with a confocal microscope (Leica SP5). Neural cells at different Z-positions within printed tissues were imaged as Z-stacks. Neural processes were counted and quantified as total outgrowth (processes emanating from both the cell bodies and from other processes) and branches (processes emanating from other processes only).

Lipid bilayer fluorescent labelling. A mixture of lipids: DPhPC (1,2-diphytanoyl-sn-glycero-3-phosphatidylcholine) and Texas Red™ 1,2-dihexadecanoyl-sn-glycero-3-phosphoethanolamine triethylammonium salt (0.1 mol %, Life Technologies, T1395MP) were added to printing oil. Printed constructs were then imaged by fluorescence confocal microscopy before and after transfer into medium.

Printing electronics. Reported droplet printing methods are limited to materials with low viscosities and low cell densities (Murphy, S. V. & Atala, A. 3D bioprinting of tissues and organs. Nature biotechnology 32, 773-785, doi:10.1038/nbt.2958 (2014)). To improve the performance of our previously described droplet printer,¹ we designed a piezo driver capable of higher output voltages. The schematic of printer electronics is shown below. Abbreviations: PC, personal computer; USB, universal serial bus; SPI, serial peripheral interface; DAC, digital-to-analog converter.

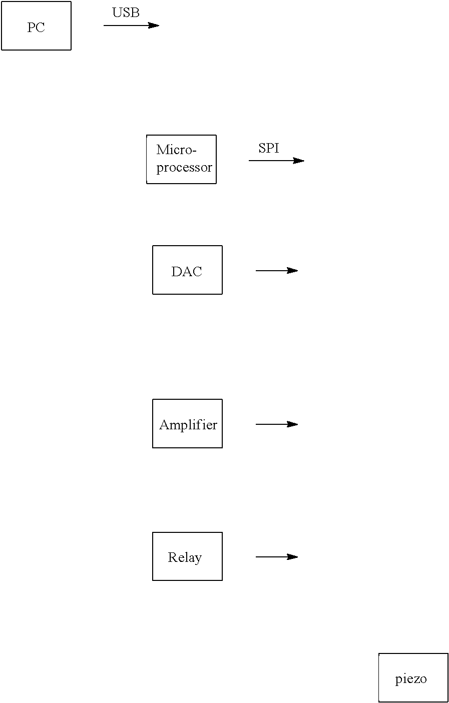

A full circuit diagram of the piezo-driver is included at the end of this document. The amplifier provides greater output voltages compared to the original design.¹ The electronics supply a voltage ranging from −130 V to +130 V under computer control. The amplifier drawing is provided by Timothy Powell (Electronic workshop, Department of Chemistry, University of Oxford).

TABLE 1
Antibodies used

| Name | Target | Species | Dilution | Supplier | Cat. No. |
|---|---|---|---|---|---|
| SOX2 | neural stem/progenitor cells | Rabbit | 1:1000 | Millipore | AB5603 |
| TUJ1 | βIII-tubulin | Guinea | 1:500 | Synaptic | 302304 |
| | Fetal and postnatal neurons | pig | | Systems | |
| CTIP2 | Cortical layers 4-6 | Rat | 1:300 | Abcam | Ab18465 |
| MAP2 | Neuronal processes | Chicken | 1:500 | Abcam | Ab5392 |
| GFAP | Astrocytes | Rat | 1:500 | Invitrogen | 13-0300 |
| NeuN | Mature neurones | Mouse | 1:400 | Millipore | MAB377 |
| Ki67 | Cycling cells | Rabbit | 1:500 | Abcam | Ab16667 |
| vGlut1 | Glutamatergic neurons | Rabbit | 1:500 | Synaptic | 135303 |
| | | | | Systems | |
| TBR1 | Cortical layers 1, 5-6, and | Rabbit | 1:300 | Abcam | Ab31940 |
| | subplate | | | | |
| SYP | Presynaptic terminals | Chicken | 1:500 | Synaptic | 101006 |
| | | | | Systems | |

Lipid Bilayer Supported Droplet Bio-Printing for Soft Tissues

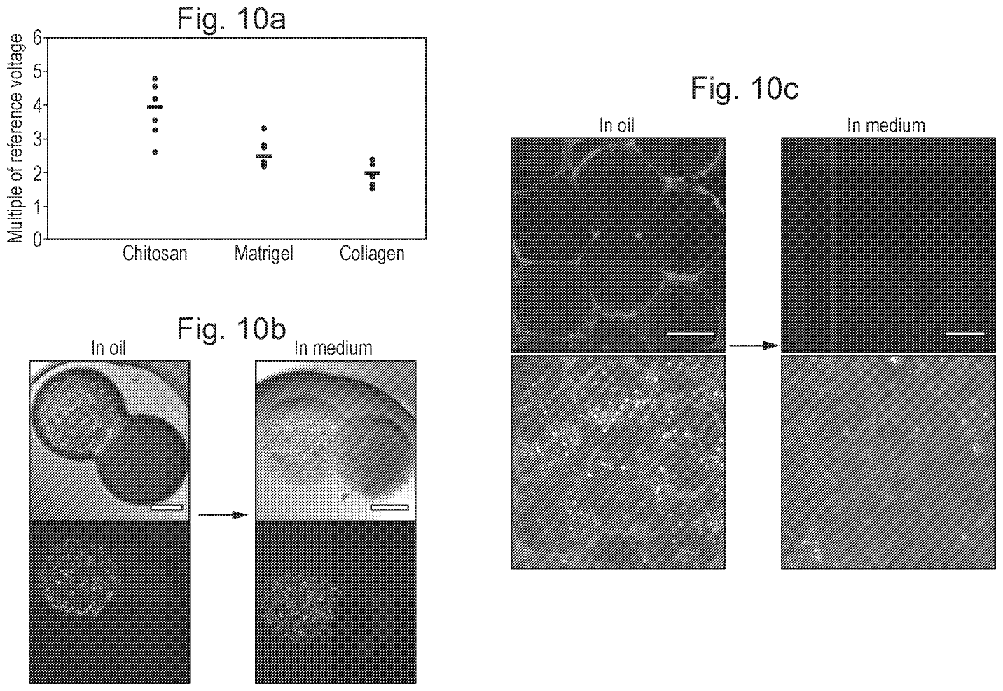

Reported droplet printing methods are limited to materials with low viscosities and low cell densities.^{[23]} To improve the performance of our previously described droplet printer,^{[19]} we designed a piezo driver capable of higher output voltages (see below). In general, higher voltage was required to eject droplets containing materials with higher viscosity (FIG. 10a). The ejected, cell-laden, ECM-containing droplets spontaneously acquired a monolayer of lipids in the lipid-containing oil bath. Droplets positioned next to each other formed droplet interface bilayers (DIBs) which provided the crucial adhesive force for supporting the 3D architecture of the printed droplet networks and allowing patterning (FIG. 1a).^{[24]} Printing nozzles with different inner diameters were prepared and used to generate droplets with different sizes (FIG. 1b). With the same nozzle, the ejected droplet size could be further tuned by adjusting the amplitude (voltage) and duration of the printing pulse. Using fluorescently-labelled lipids, we also confirmed that DIBs were present between droplets (FIG. 1c), enabling compartmentalisation.^{[19, 24]} Thus, the droplets are separated from each other and do not exchange their ECM or cell contents during the printing process.

After printing, the structures were gelated by warming to room temperature (RT), during which the intact DIBs kept the droplet contents separated (FIG. 1d, upper row). Matrigel gelation is slow at RT, whereas warming DIBs to 37° C. results in rapid rupture and droplet fusion (FIG. 1d, middle row). Rupture of DIBs can also occur when the printing oil is exchanged with culture medium for post-printing culture. It is therefore essential to replace the DIBs with gelated ECM between the droplets to maintain the 3D architecture of the droplet network. To achieve this, we used a stepped temperature increase protocol. The printed network is first warmed to an intermediate temperature of ˜25° C. for 30 min, whereby partial gelation of Matrigel occurs, but where also the DIBs remain intact and maintain separation of droplet contents (FIG. 1d, upper row). The network is subsequently incubated at 37° C., whereupon the DIBs break and further gelation occurs across the former interface, leading to droplet connection without fusion (FIG. 1d, bottom row). The droplet network could then be transferred from the printing oil to culture medium without deformation or exchange of the cell contents between droplets (FIG. 1e, FIG. 10b). The fluorescence of the labelled DIBs slowly disappeared from the networks within a few days while in culture (FIG. 10c), indicating that lipids had diffused away. Thus, ECM and cells were left behind without the need for any materials with stiffness above 1 kPa for mechanical support. Therefore, this technique allows the pre-patterning of cells in scaffold-free soft ECM, avoiding the addition of biologically incompatible materials. Further, the DIBs provide stabilization during the several hours required for materials such as Matrigel to gelate.

Printed Tissues Recapitulated Cortical Development Events.

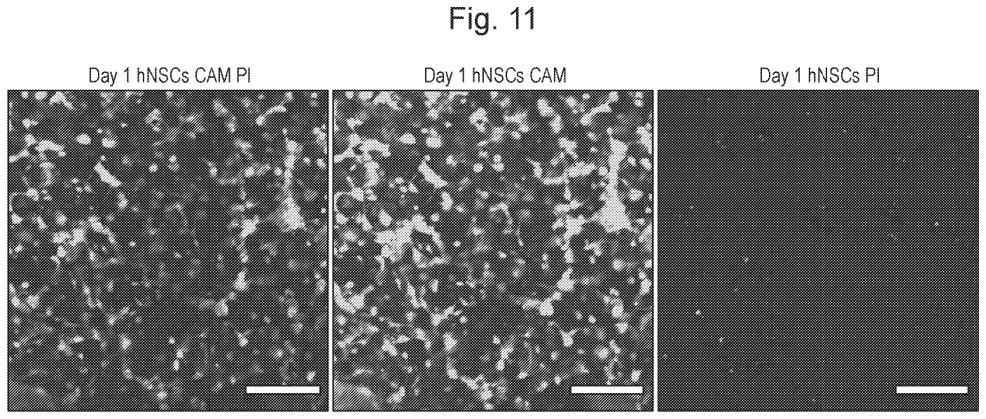

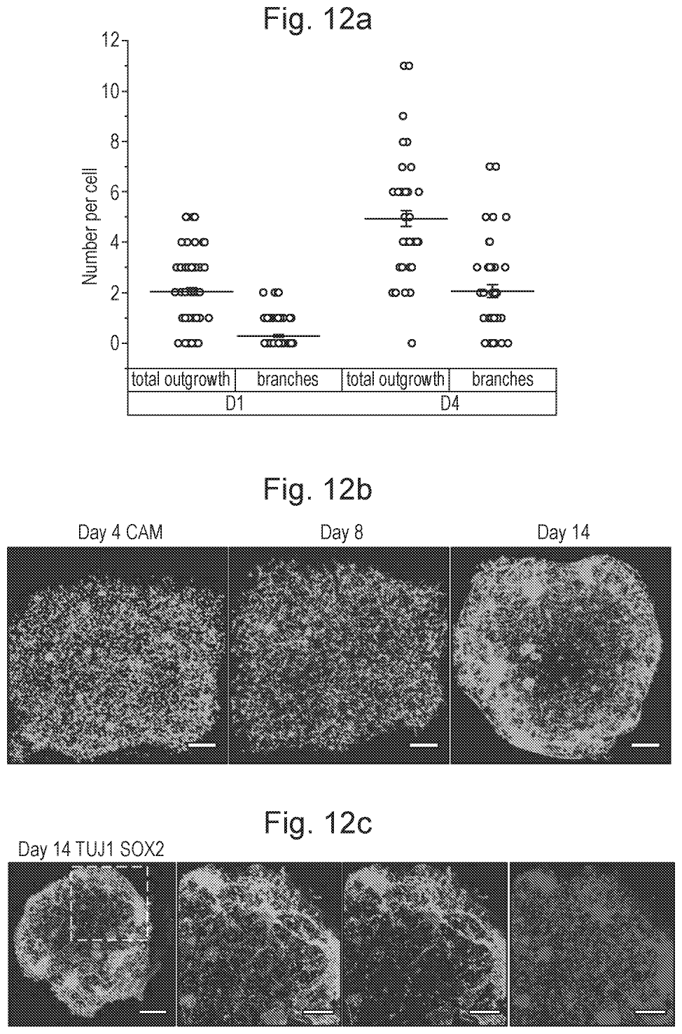

We next investigated whether we could construct soft human neural tissues using our printing technique. First, we performed 2D culture of hiPSC-derived NSCs in neural expansion medium with FGF2 and EGF for 4 to 7 d (FIG. 2a). The hNSCs were then harvested and suspended in Matrigel at a cell density of 2×10⁷ mL⁻¹. After printing with this ‘bio-ink’, the constructed neural tissues (printed as 7×7×4 droplet networks) were cultured in neural maintenance medium (NMM) for differentiation. At day 1 post-printing differentiation (ppd), the neural cells started to polarise, producing processes (FIG. 2b). The neural cells projected processes across the former droplet boundaries into the adjacent droplets. The number of neural process outgrowths and branches increased over the first few days of culture (FIG. 12a) and more neural morphological changes appeared over longer differentiation times (FIG. 2b, FIG. 12b). Notably, the printed cells exhibited high viability (˜96%), indicating that stress from the printing process had been limited (FIG. 2c, FIG. 11). Immunostaining for the young neuron marker 111-tubulin with TUJ1 antibodies revealed that neuronal processes extended over the first two weeks of differentiation (FIG. 2d). The distribution of cells at day 14 ppd was heterogeneous, with TUJ1⁺ neurons predominantly found at the surface of a construct whereas SOX2⁺ neural progenitor cells were present throughout the whole construct (FIG. 12c).

We further studied the self-organisation of neural cells in printed tissues by increasing the cell density to 3.5×10⁷ mL⁻¹. Constructs printed at high cell density exhibited high viability and the cells were able to proliferate, resulting in dense structures (FIG. 2e) by day 28 ppd. The high cell density also triggered SOX2⁺ TUJ1⁺ neural rosette formation at day 28 ppd (FIG. 2e, f). Longer culture led to the formation of protuberances and an increasing number of deep cortical layer neurons (CTIP2⁺, FIG. 2g). Further, mature neurons (MAP2⁺ and NeuN⁺) and astrocytes (GFAP⁺) appeared after ˜2 months of differentiation (FIG. 2h). Printed neural tissues were kept for over 100 days (FIG. 13). These data demonstrate the construction of viable neural tissues with different cell densities, and their self-organisation and differentiation over long time periods.

Functional Tissue Constructs have Connected Neural Networks

Calcium ions (Ca²⁺) are necessary in many cellular processes and serve as intracellular second messengers.^{[25]} Spontaneous Ca²⁺ oscillations have been suggested to play a role in brain development.^{[26]} Further, synchronised Ca²⁺ oscillations of interconnected neuronal networks are essential for brain functions.^{[27]} To test the functionality of the neurons in our printed constructs, we performed Ca²⁺ imaging with Fluo-4. Time-lapse recordings revealed spontaneous Ca²⁺ oscillations in printed constructs after 4 days ppd (FIG. 3a). Individual cell traces showed irregular patterns of calcium spikes (FIG. 3b). Correlation coefficients were quantified and revealed low correlation between printed neural cells at an early differentiation stage (4 days ppd, FIG. 3c). However, the cells exhibited more frequent and more regular Ca²⁺ oscillations at day 44 ppd after further differentiation in culture (FIGS. 3d and e) and correlation coefficients revealed significantly higher connectivity at this stage of differentiation (FIG. 3f). To determine whether printed neural tissues could respond to stimulation, we exposed the constructs with 65 days ppd to KCl (60 mM), which evoked rapid Ca²⁺ transients (FIG. 3g-i).

Pre-Patterning Revealed Cell-Autonomous Process Outgrowth and Cell Migration

Neural cell process outgrowth and cell migration are key features in the development of the central nervous system.^{[28]} In particular, apical NSCs (radial glia) grow long processes and migrate away from the interior of the developing brain to become basal progenitors and outer radial glia. Additionally, neurons born in the interior extend processes and migrate towards the exterior. To test whether printed tissues could recapitulate these cortex developmental events, we designed a printing scheme (FIG. 4a) comprising interior hNSCs (orange) encased in a cell-free Matrigel exterior compartment (blue), hNSCs (in)-Matrigel (out). The interior neural cells projected processes into the exterior Matrigel the first a few days of culture in differentiation medium. The length of the processes increased overtime, and was followed by cell migration from the hNSC compartment to the Matrigel compartment (FIGS. 4b and c). Using RFP labelled hNSCs (RFP-hNSCs), we further confirmed the process projection and the migration of RFP-hNSCs into the initially cell-free Matrigel compartment (FIG. 4c). At day 28 ppd, neurons had filled the Matrigel compartment and immunostaining revealed the presence of progenitors (SOX2⁺), young neurons (TUJ1⁺) and deep layer cortical neurons (CTIP2⁺, FIG. 4d). Further, staining for TBR1 revealed the differentiation of sub-plate neurons at day 56 ppd (FIG. 4e). Astrogenesis also occurred at this time in the previously cell-free compartment together with differentiated neurons (MAP2⁺) and proliferating cells (Ki67⁺). These data show that the interior printed cells were able to extend processes and migrate into the cell free exterior, recapitulating aspects of cortical development in a cell autonomous manner.

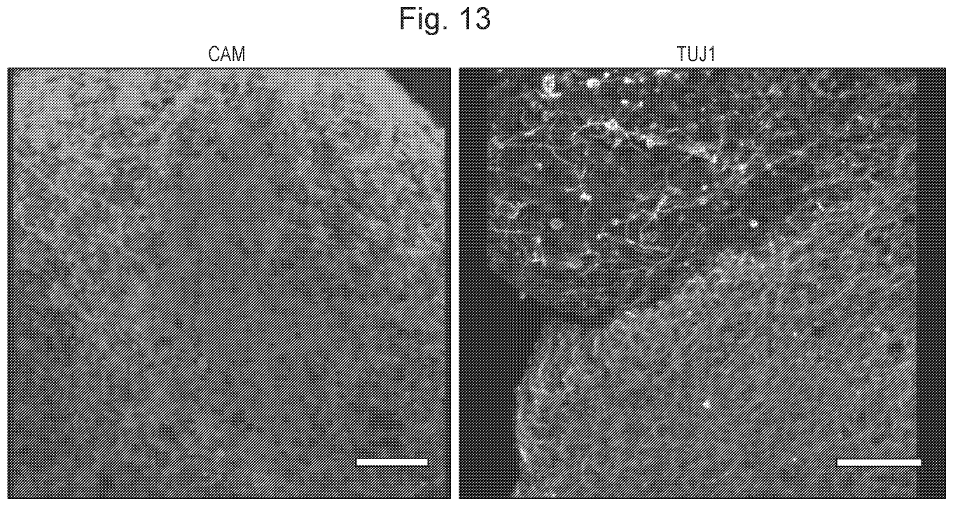

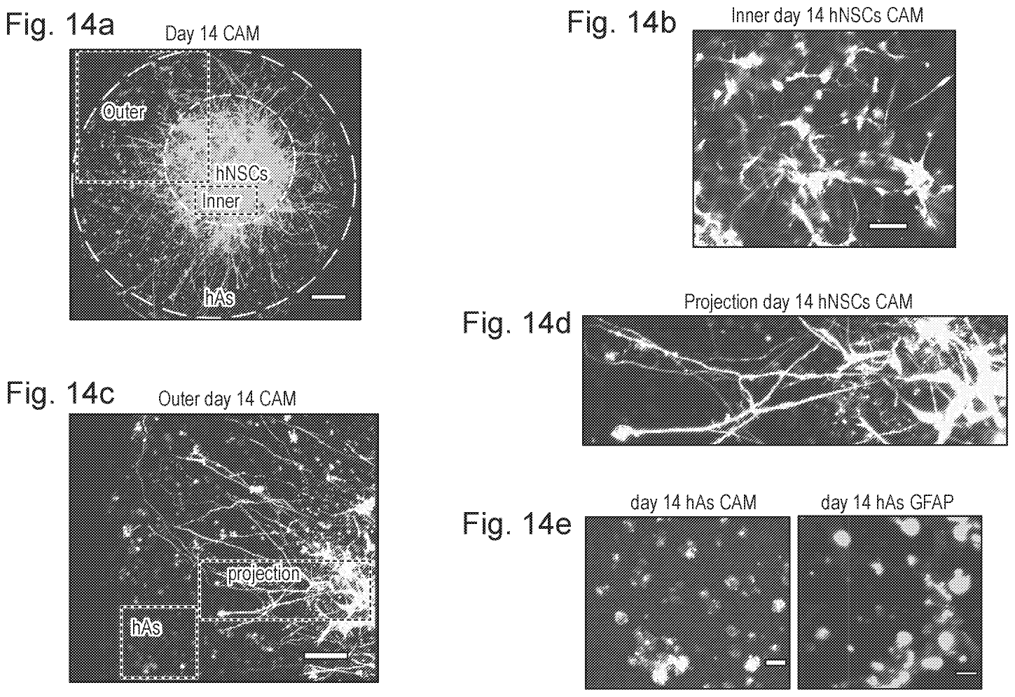

Since astrocytes regulate axon outgrowth and cell migration in adult neurogenic niches and in pathology, we hypothesised that they may play a role in these events during neurodevelopment. To address this question and test if astrocytes printed together with Matrigel in the exterior compartment affect the above results, we 3D printed primary human cerebral cortex astrocytes around hNSCs. hNSCs and human astrocytes (hAs) were printed in an hNSCs (in)-hAs (out) pattern with a higher density of hNSCs (3.5×10⁷ mL⁻¹) than hAs (1.0×10⁷ mL⁻¹) to model the early stage of astrogenesis (FIG. 4f). Similar to the progression observed with hNSCs (in)-Matrigel (out), interior neural cells projected long processes into the exterior hAs compartment, which was followed by neural cell migration (FIG. 4g, 4h and FIG. 14). The neural cells distributed themselves throughout the network with many moving from the interior to the exterior compartment. Unexpectedly, we did not observe hAs in the interior compartment by day 28 ppd. The hAs not only remained in the exterior compartment but moved to the surface of the constructs (FIG. 4h). These results suggest astrocyte positioning may be restricted by the interior compartment, which was primarily neuronal.

Astrocytes Induce Axonal Fasciculation in Patterned Cortical Tissues

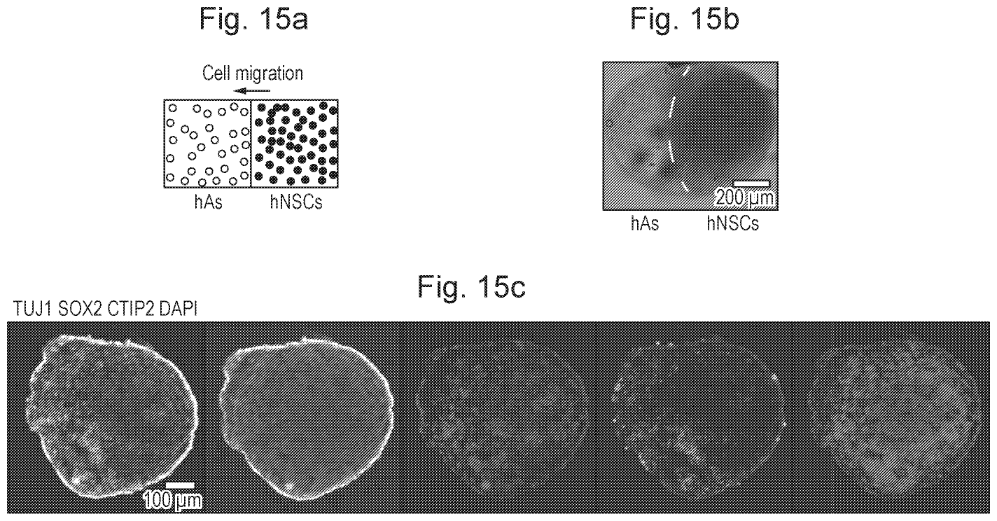

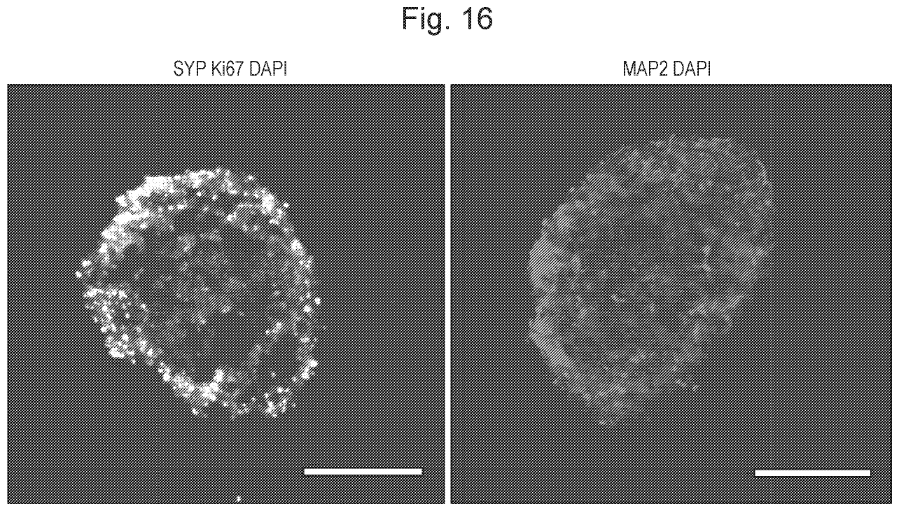

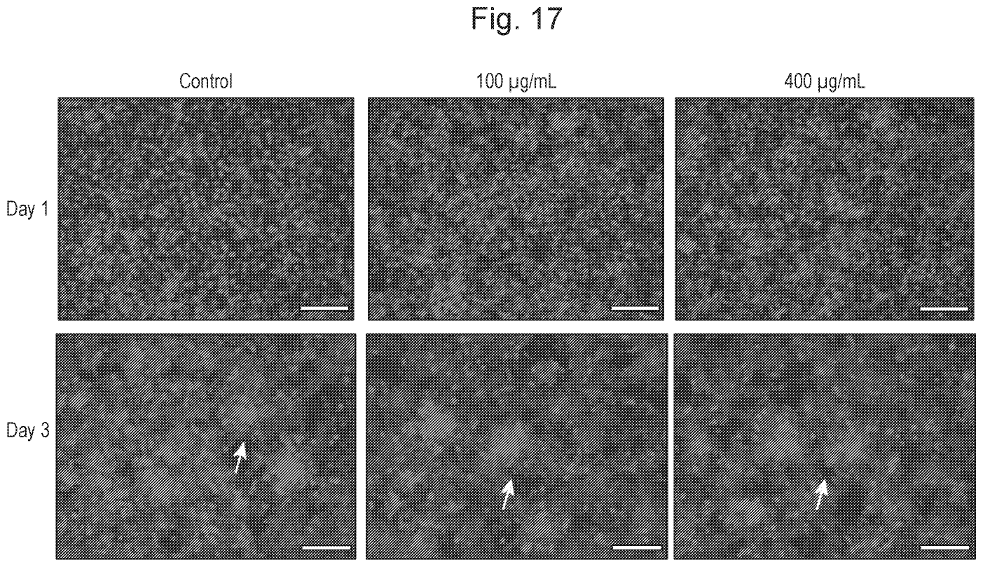

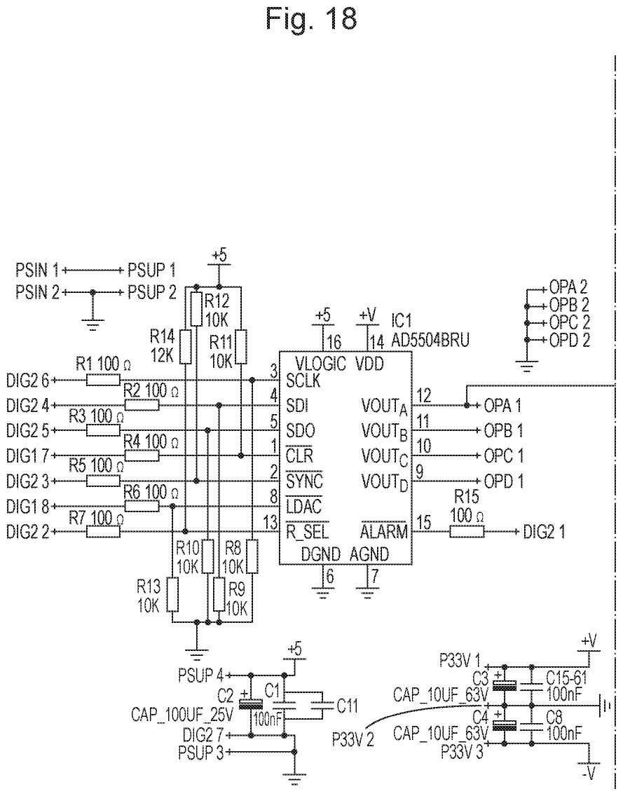

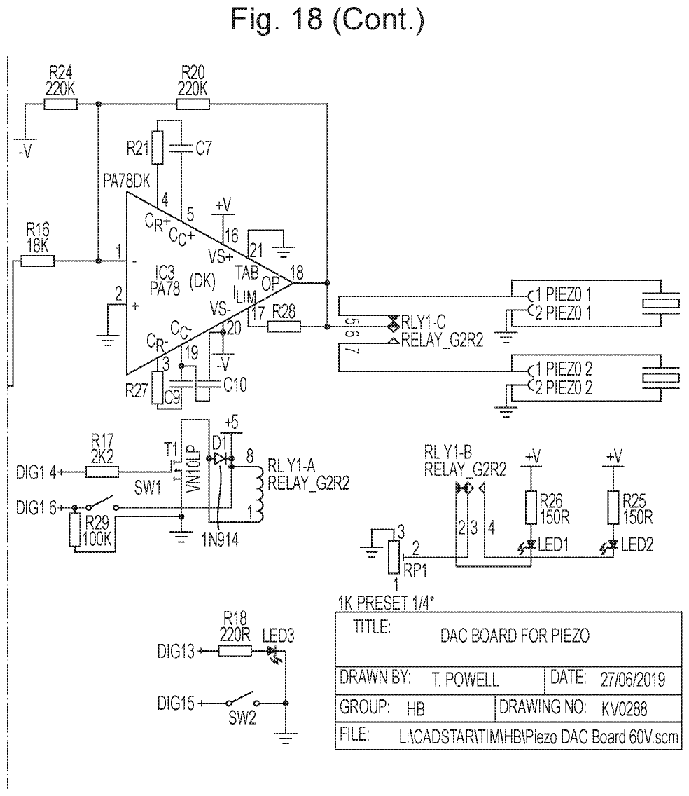

Using confocal microscopy, we re-constructed Z-stack images of day 28 ppd hNSCs (in)-Matrigel (out) and hNSCs (in)-hAs (out) printed tissues. Unexpectedly, we observed the formation of neural process bundles in the hNSCs (in)-hAs (out) tissues, but not in the hNSCs (in)-Matrigel (out) tissues (FIGS. 5a and b). This was not due to preferential axonal growth into the hAs-containing domains since the TUJ1⁺ neuronal processes grew equally well into cell-free Matrigel or astrocyte-containing exterior compartments (FIG. 5c). The process bundles formed in the hAs compartment had an average width of 65±22 μm (n=7) comparing to 5±3 μm (n>7) in Matrigel only compartment (FIG. 5d). These data suggest astrocyte-induced neural fasciculation, which was previously observed in mice.^{[29]} To confirm this, we also printed a hAs (left)-hNSCs (right) pattern and found that hNSCs projected processes towards the hAs compartment (FIG. 5e). Moreover, progenitors (SOX2⁺) aligned along the bundled processes indicating axon-guided cell migration (FIGS. 5e and 5f). The majority of the migrating cells subtend an angle of <200 with an associated process bundle (FIG. 5g). Although the starting density of the hAs (1.0×10⁷ mL⁻¹) was less than the hNSCs (3.5×10⁷ mL⁻¹), similar neural cell density was observed in both compartments at day 28 ppd (FIG. 15), suggesting neural cell migration into the astrocyte compartment. Notably, TUJ1⁺ neuronal processes appeared to be preferentially bundled in the hAs compartment but not in the NSCs compartment (FIG. 5f), again suggesting astrocyte-mediated neural fasciculation. Our data show that astrocytes exert a profound effect on the late developmental event of axonal bundling.

Fast Production of Differentiated Cortical Tissues

Recent advances in rapid cell programming allow the production of mature neurons and astrocytes in just a few weeks.^{[21, 22, 30]} These fast programming methods may provide important sources of cells for 3D printing of mature brain tissues quicker than classic reprogramming. We first tested whether we could construct viable differentiated cortical tissues by using rapidly matured human cortical neurons (hCNs) with hAs. Because the hCNs do not proliferate in 2D culture and cannot be harvested without damage after seeding, we added them directly into the bio-ink after thawing (FIG. 6a). We first 3D printed homogeneously mixed hCNs and hAs at a density of 1.75×10⁷ mL⁻¹ for each cell types. After 13 d, printed hCNs had produced long axons (FIG. 6b). These mature neurons also expressed MAP2, the glutamatergic neural marker vGlut1, and the synaptic marker SYP (FIG. 16). Both hCNs and hAs retained their homogenous distribution throughout the printed constructs during 13 days in culture.

Studies in mouse models suggest that cortical astrocytes are domain-specific and restricted to their birth place, and cannot migrate even after injury.^{[31, 32]} Therefore, to investigate human astrocyte migration, we pre-printed neurons and astrocytes in spatially segregated compartments, either hCNs (in)-hAs (out) or the reverse (FIG. 6c). In the hCNs (in)-hAs (out) constructs, the hCNs migrated to the astrocyte compartment after less than two weeks, whereas hAs mostly remained in the outer compartment with a small fraction, ˜7%, having migrated to the centre of the constructs (FIGS. 6c and d). This is similar to and supportive of our data in FIG. 4f-h showing neural migration from the interior to the exterior astrocyte-containing compartment, whereas astrocytes remained in the outer compartment. In the hAs (in)-hCNs (out) constructs, ˜80% of the hAs remained in the centre of the structure (FIG. 6d). Thus in both pre-patterned constructs, hAs maintained their segregation from neurons but not vice-versa, showing a non-reciprocal functional interaction.

Discussion

One of the most powerful tools in developmental biology is heterotopic and heterochronic transplantation of tissues, such as Spemann and Mangold's seminal work that uncovered the neural organiser.^{[33]} Juxtaposition of tissue in artificial patterns allowed the eventual discovery of molecular mechanisms and signalling pathways necessary for development. However, it has been a challenge to juxtapose small clusters of distinct cells to ask more fine-grained questions, until the advent of 3D bio-printing. Astrocytes are well-distributed in different layers of cortex and in close contact with neurons.^{[34]} By pre-positioning astrocytes and cortical neurons with different patterns, we demonstrated that neurons readily migrate into astrocyte domains but that astrocytes prefer to remain segregated from neurons. These observations are consistent with previous reports that astrocytes have limited capacity for migration from their birth locations. However, by experimentally pre-positioning astrocytes separately but next to NSCs, we surprisingly observed that astrocytes assist axonal bundling which is a prelude to tract formation. With the future development of hiPSC-derived region-specific brain cells, different combinations of precursor and mature cells can be assembled through bio-printing to probe local or distant cross-region brain activities and malfunctions. For example, deep and upper layers of cortical cells might be printed in layered structures to study cell migration across different cortical layers.

To achieve these biological insights, we developed a technique that can be used to print a variety of soft tissues with ECM and without the need to add hard materials. Our technique could potentially be applied to other matrices, such as organ/tissue-derived or artificial ECM, to provide tailored microenvironments in printed 3D tissues. The fabrication of tissue constructs with physiologically relevant cellular microenvironments is crucial for the study of tissues with limited accessibility, such as the human brain. By using Matrigel as the sole printing matrix, we have demonstrated that 3D bio-printing can be applied to the production of cortical tissues without affecting the viability, function and self-organisation of the incorporated cells. Bio-printing assigns specific cell types to pre-determined initial positions, unlike the process of organoid formation. This spatial pre-patterning not only gives better control over self-organisation processes, but also allows the generation of unnatural patterns allowing interrogation of the underlying mechanisms of self-organisation.

Finally, we demonstrated that by combining our technique with fast neuron programming, differentiated cortical tissues can be constructed within weeks, whereas organoids take months to develop. Although organoids can yield most brain cell types following developmental progression, later-born cells can take months or more to reach significant numbers. Indeed, 2D culture still has an advantage over 3D culture for the mass production of homogeneous cells. However, by taking advantage of advances in the 2D culture of diverse brain cell types, brain tissues from later stages of differentiation might be generated through pre-patterned constructs in a fast manner. Together, our data suggest that 3D bio-printing can be applied to spatially position distinct cells to construct 3D tissue models and guide self-organisation. The approach can be applied to study human brain developmental processes such as cortical expansion and astrocyte migration/segregation. Finally, diseases could also be modelled by incorporating reprogrammed patient cells with specific genetic mutations.

### REFERENCES

[1] C. M. Nelson, M. J. Bissell, Of extracellular matrix, scaffolds, and signaling: Tissue architecture regulates development, homeostasis, and cancer, Annu Rev Cell Dev Bi 22 (2006) 287-309.
[2] Y. C. Shi, P. Kirwan, J. Smith, H. P. C. Robinson, F. J. Livesey, Human cerebral cortex development from pluripotent stem cells to functional excitatory synapses, Nat Neurosci 15(3) (2012) 477-U180.
[3] M. A. Lancaster, M. Renner, C. A. Martin, D. Wenzel, L. S. Bicknell, M. E. Hurles, T. Homfray, J. M. Penninger, A. P. Jackson, J. A. Knoblich, Cerebral organoids model human brain development and microcephaly, Nature 501(7467) (2013) 373-+.
[4] N. D. Amin, S. P. Pasca, Building Models of Brain Disorders with Three-Dimensional Organoids, Neuron 100(2) (2018) 389-405.
[5] A. Warmflash, B. Sorre, F. Etoc, E. D. Siggia, A. H. Brivanlou, A method to recapitulate early embryonic spatial patterning in human embryonic stem cells, Nat Methods 11(8) (2014) 847-854.
[6] G. T. Knight, B. F. Lundin, N. Iyer, L. M. T. Ashton, W. A. Sethares, R. M. Willett, R. S. Ashton, Engineering induction of singular neural rosette emergence within hPSC-derived tissues, Elife 7 (2018).
[7] F. Birey, J. Andersen, C. D. Makinson, S. Islam, W. Wei, N. Huber, H. C. Fan, K. R. C. Metzler, G. Panagiotakos, N. Thom, N. A. O'Rourke, L. M. Steinmetz, J. A. Bernstein, J. Hallmayer, J. R. Huguenard, S. P. Pasca, Assembly of functionally integrated human forebrain spheroids, Nature 545(7652) (2017) 54-+.
[8] G. Y. Cederquist, J. J. Asciolla, J. Tchieu, R. M. Walsh, D. Cornacchia, M. D. Resh, L. Studer, Specification of positional identity in forebrain organoids, Nature biotechnology 37(4) (2019) 436-+.
[9] H. W. Kang, S. J. Lee, I. K. Ko, C. Kengla, J. J. Yoo, A. Atala, A 3D bioprinting system to produce human-scale tissue constructs with structural integrity, Nature biotechnology 34(3) (2016) 312-9.
[10] T. J. Hinton, Q. Jallerat, R. N. Palchesko, J. H. Park, M. S. Grodzicki, H. J. Shue, M. H. Ramadan, A. R. Hudson, A. W. Feinberg, Three-dimensional printing of complex biological structures by freeform reversible embedding of suspended hydrogels, Science advances 1(9) (2015) e1500758.
[11] M. T. Poldervaart, H. Gremmels, K. van Deventer, J. O. Fledderus, F. C. Oner, M. C. Verhaar, W. J. Dhert, J. Alblas, Prolonged presence of VEGF promotes vascularization in 3D bioprinted scaffolds with defined architecture, Journal of controlled release: official journal of the Controlled Release Society 184 (2014) 58-66.
[12] B. A. Nerger, P.-T. Brun, C. M. Nelson, Microextrusion Printing Cell-Laden Networks of Type I Collagen with Patterned Anisotropy and Geometry BioRxiv http://dx.doi.org/10.1101/509265 (2019).
[13] Q. Gu, E. Tomaskovic-Crook, R. Lozano, Y. Chen, R. M. Kapsa, Q. Zhou, G. G. Wallace, J. M. Crook, Stem Cell Bioprinting: Functional 3D Neural Mini-Tissues from Printed Gel-Based Bioink and Human Neural Stem Cells (Adv. Healthcare Mater. 12/2016), Advanced healthcare materials 5(12) (2016) 1428.
[14] Q. Gu, E. Tomaskovic-Crook, G. G. Wallace, J. M. Crook, 3D Bioprinting Human Induced Pluripotent Stem Cell Constructs for In Situ Cell Proliferation and Successive Multilineage Differentiation, Advanced healthcare materials 6(17) (2017).
[15] R. Lozano, L. Stevens, B. C. Thompson, K. J. Gilmore, R. Gorkin, 3rd, E. M. Stewart, M. in het Panhuis, M. Romero-Ortega, G. G. Wallace, 3D printing of layered brain-like structures using peptide modified gellan gum substrates, Biomaterials 67 (2015) 264-73.
[16] R. Sharma, I. P. M. Smits, L. De La Vega, C. Lee, S. M. Willerth, 3D Bioprinting Pluripotent Stem Cell Derived Neural Tissues Using a Novel Fibrin Bioink Containing Drug Releasing Microspheres, Front Bioeng Biotechnol 8 (2020) 57.
[17] D. Joung, V. Truong, C. C. Neitzke, S. Z. Guo, P. J. Walsh, J. R. Monat, F. B. Meng, S. H. Park, J. R. Dutton, A. M. Parr, M. C. McAlpine, 3D Printed Stem-Cell Derived Neural Progenitors Generate Spinal Cord Scaffolds, Adv Funct Mater 28(39) (2018).
[18] F. Y. Hsieh, H. H. Lin, S. H. Hsu, 3D bioprinting of neural stem cell-laden thermoresponsive biodegradable polyurethane hydrogel and potential in central nervous system repair, Biomaterials 71 (2015) 48-57.
[19] G. Villar, A. D. Graham, H. Bayley, A tissue-like printed material, Science 340(6128) (2013) 48-52.
[20] A. D. Graham, S. N. Olof, M. J. Burke, J. P. K. Armstrong, E. A. Mikhailova, J. G. Nicholson, S. J Box, F. G. Szele, A. W. Perriman, H. Bayley, High-Resolution Patterned Cellular Constructs by Droplet-Based 3D Printing, Sci Rep-Uk 7 (2017).
[21] M. Pawlowski, D. Ortmann, A. Bertero, J. M. Tavares, R. A. Pedersen, L. Vallier, M. R. N. Kotter, Inducible and Deterministic Forward Programming of Human Pluripotent Stem Cells into Neurons, Skeletal Myocytes, and Oligodendrocytes, Stem Cell Rep 8(4) (2017) 803-812.
[22] Y. S. Zhang, C. Pak, Y. Han, H. Ahlenius, Z. J. Zhang, S. Chanda, S. Marro, C. Patzke, C. Acuna, J. Covy, W. Xu, N. Yang, T. Danko, L. Chen, M. Wernig, T. C. Sudhof, Rapid Single-Step Induction of Functional Neurons from Human Pluripotent Stem Cells, Neuron 78(5) (2013) 785-798.
[23] S. V. Murphy, A. Atala, 3D bioprinting of tissues and organs, Nature biotechnology 32(8) (2014) 773-85.
[24] H. Bayley, B. Cronin, A. Heron, M. A. Holden, W. L. Hwang, R. Syeda, J. Thompson, M. Wallace, Droplet interface bilayers, Molecular bioSystems 4(12) (2008) 1191-208.
[25] A. B. Toth, A. K. Shum, M. Prakriya, Regulation of neurogenesis by calcium signaling, Cell Calcium 59(2-3) (2016) 124-134.
[26] M. Brini, T. Cali, D. Ottolini, E. Carafoli, Neuronal calcium signaling: function and dysfunction, Cell Mol Life Sci 71(15) (2014) 2787-2814.
[27] Y. Penn, M. Segal, E. Moses, Network synchronization in hippocampal neurons, P Natl Acad Sci USA 113(12) (2016) 3341-3346.
[28] R. Ayala, T. Z. Shu, L. H. Tsai, Trekking across the brain: The journey of neuronal migration, Cell 128(1) (2007) 29-43.
[29] S. Minocha, D. Valloton, A. R. Ypsilanti, H. Fiumelli, E. A. Allen, Y. Yanagawa, O. Marin, A. Chedotal, J. P. Hornung, C. Lebrand, Nkx2.1-derived astrocytes and neurons together with Slit2 are indispensable for anterior commissure formation, Nat Commun 6 (2015).
[30] I. Canals, A. Ginisty, E. Quist, R. Timmerman, J. Fritze, G. Miskinyte, E. Monni, M. G. Hansen, I. Hidalgo, D. Bryder, J. Bengzon, H. Ahlenius, Rapid and efficient induction of functional astrocytes from human pluripotent stem cells, Nat Methods 15(9) (2018) 693-+.
[31] H. H. Tsai, H. L. Li, L. C. Fuentealba, A. V. Molofsky, R. Taveira-Marques, H. L. Zhuang, A. Tenney, A. T. Mumen, S. P. J. Fancy, F. Merkle, N. Kessaris, A. Alvarez-Buylla, W. D. Richardson, D. H. Rowitch, Regional Astrocyte Allocation Regulates CNS Synaptogenesis and Repair, Science 337(6092) (2012) 358-362.
[32] S. Magavi, D. Friedmann, G. Banks, A. Stolfi, C. Lois, Coincident Generation of Pyramidal Neurons and Protoplasmic Astrocytes in Neocortical Columns, J Neurosci 32(14) (2012) 4762-4772.
[33] H. Spemann, H. Mangold, Induction of embryonic primordia by implantation of organizers from a different species. 1923, Int J Dev Biol 45(1) (2001) 13-38.
[34] N. A. Oberheim, T. Takano, X. Han, W. He, J. H. C. Lin, F. Wang, Q. Xu, J. D. Wyatt, W. Pilcher, J. G. Ojemann, B. R. Ransom, S. A. Goldman, M. Nedergaard, Uniquely Hominid Features of Adult Human Astrocytes, J Neurosci 29(10) (2009) 3276-3287.
[35] W. Haenseler, S. N. Sansom, J. Buchrieser, S. E. Newey, C. S. Moore, F. J. Nicholls, S. Chintawar, C. Schnell, J. P. Antel, N. D. Allen, M. Z. Cader, R. Wade-Martins, W. S. James, S. A. Cowley, A Highly Efficient Human Pluripotent Stem Cell Microglia Model Displays a Neuronal-Co-culture-Specific Expression Profile and Inflammatory Response, Stem Cell Rep 8(6) (2017) 1727-1742.
[36] J. Schindelin, I. Arganda-Carreras, E. Frise, V. Kaynig, M. Longair, T. Pietzsch, S. Preibisch, C. Rueden, S. Saalfeld, B. Schmid, J. Y. Tinevez, D. J. White, V. Hartenstein, K. Eliceiri, P. Tomancak, A. Cardona, Fiji: an open-source platform for biological-image analysis, Nat Methods 9(7) (2012) 676-82.

### Example 2—Droplet Arrays

Printing Droplet Arrays with Cells in ECM.

We printed low picoliter to nanoliter droplets with cells in ECM, Matrigel or collagen using the printing techniques described herein. Microfluidics are difficult to generate low picoliter droplets (below 30 um) with viscous materials.¹ Cole et al. produced cell droplets in PBS, without matrix.² Our method also allows low sample loading (a few microliters for our method vs >200 uL for microfluidics), which is important for precious samples. We also show comparable droplet size distribution compared to microfluidics.

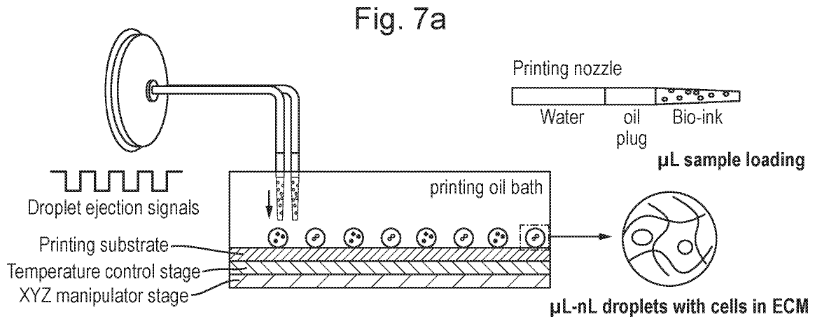

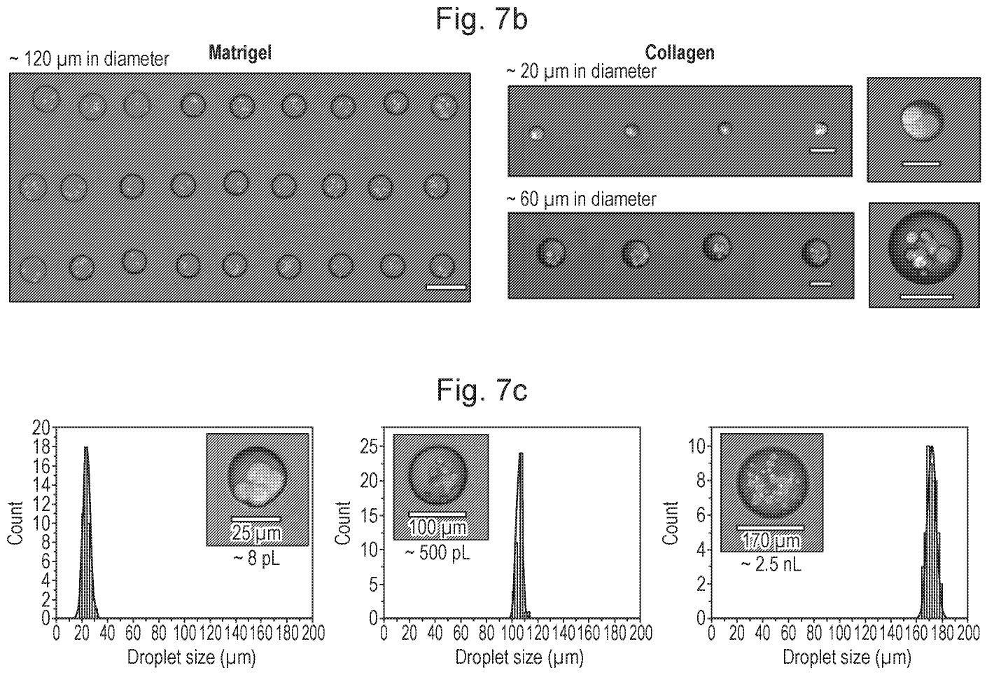

FIG. 7 shows the results for printing arrays of picoliter and nanolitre droplets with cells in ECM. FIG. 7a, Printer setup for printing droplet arrays. Droplets were ejected through printer nozzles under the control of computer designed electric signals. Printing sample was loaded into the printing nozzle and separated by an oil plug from the water, which transduced the propelling force induced by the electric signals. Low microliters of bio-ink were needed with this setup. Printing oil bath consisted of a mixture of silicon oil and undecane. Printing container was placed on a temperature-controlled stage and XYZ manipulator stage. Printed droplets contain both cells and ECM and are from low picoliter to nanolitre in volume. FIG. 7b, An example of printed droplet array containing MDA cells (1×10⁷/mL) in Matrigel. The printed droplets are ˜120 μm in diameter. FIG. 7c, Two examples of printed droplet arrays containing two cell types in collagen. The same bio-ink was used to produce droplets with a diameter of either ˜20 (top) or ˜60 (bottom) μm. Bio-ink: HepG2 cells with green (8×10⁶/mL) and far-red (4×10⁶/mL, false coloured as magenta) cell tracker in collagen. FIG. 7d, Size distribution of printed droplets with different sizes. Scale bars are: 200 μm in FIG. 7b and 50 μm in FIG. 7c, except the top right image, which is 20 μm.

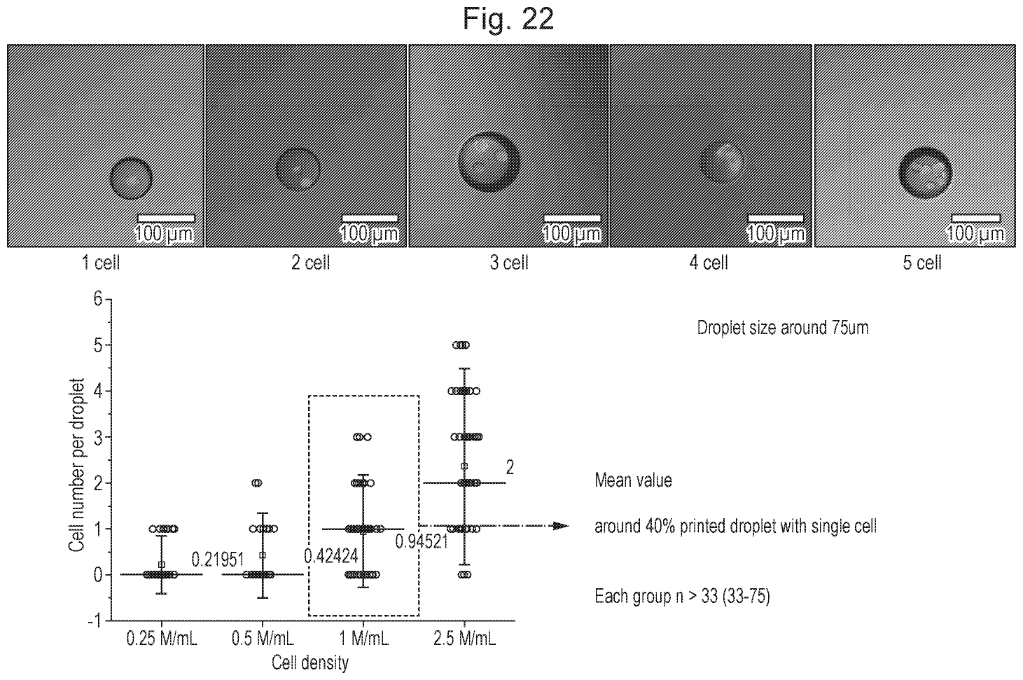

FIG. 22 shows the results from printing droplets with defined number of cells in ECM. Top row, from left to right: printed droplets containing 1, 2, 3, 4 and 5 cells respectively. Droplets are around 75 μm in diameter. Bottom graph shows the distribution of cell numbers in droplets at different cell density of the bio-ink: 0.25, 0.5, 1 and 2.5 million/mL. ˜40% of the droplets at 1 million/mL containing single cell. This could be combined with a droplet sorter to achieve printing with cell number defined droplets.

The Production of Patterned Droplet Arrays

We used sequential printing to generate patterned droplet assemblies containing 2-3 droplets with different cell content. Microfluidics relied on the use of alginate to fabricate patterned droplets (a non-biocompatible material).³ We demonstrated that various patterns could be generated with this method by controlling droplet number, droplet sizes and 3D arrangement of the droplets.

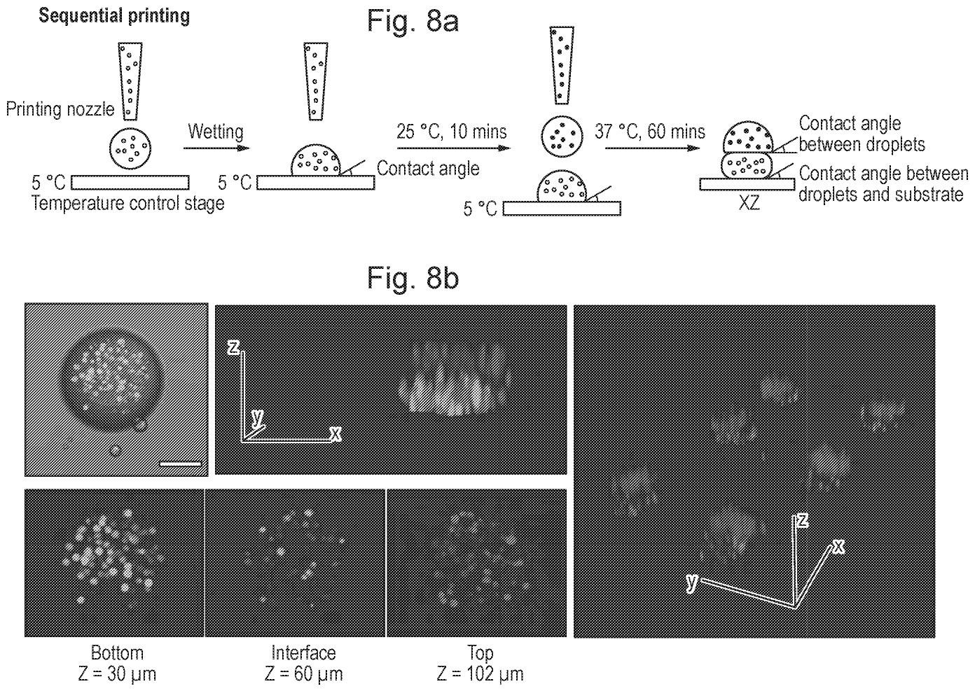

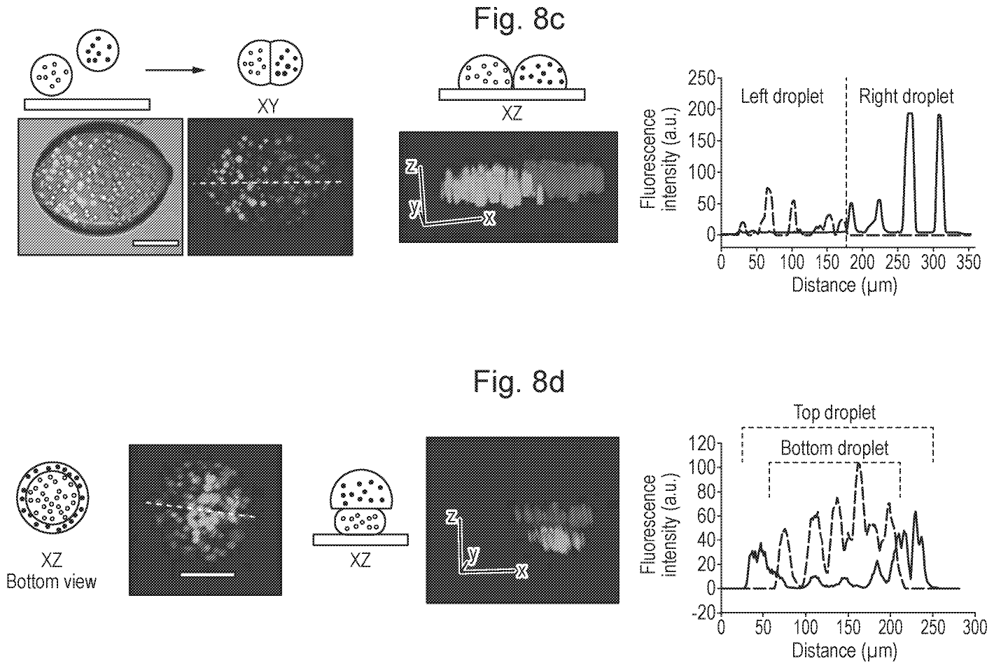

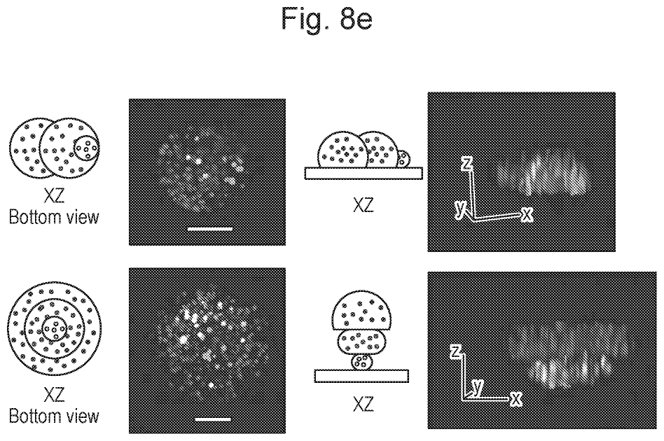

FIG. 8 shows the results for the production of patterned droplet arrays. FIG. 8a, Schematic of sequential printing to generate top-bottom patterned droplet arrays. First droplet (green) was print at 5° C. and immediately formed contact angle with the printing substrate. The droplet was then incubation at 25° C. for 10 mins for partial gelation before the temperature was cooled back to 5° C. The second droplet was printed on top of the first partially gelated (sticky) droplet to produce the desired top-bottom pattern. Patterned droplets were then incubated at 37° C. for 60 mins. Notably, the first droplet formed contact angle with both substrate and the second droplet. FIG. 8b, Confocal fluorescent images of top-bottom patterned droplet arrays. Left: an example of the top-bottom patterned droplet pair with bottom image (top left), reconstructed 3D image through z-stack (top right) and images at different confocal planes (bottom three images). Right: Constructed 3D image of an array of top-bottom patterned droplet pairs. FIG. 8c, Constructed left-right patterned droplet pairs. Left: schematic of left-right patterned droplet pair from XY dimensions with a bottom view (top) and images of the patterned droplet pair (bottom). The dashed line indicates the location of the fluorescence prolife in the chart (right). Middle: schematic of left-right patterned droplet pair from XZ dimensions (top) and the 3D reconstructed image. Right, profile diagram of both green and red fluorescence intensity along the dashed line (left). Sharp droplet boundary is observed in the centre of the diagram. FIG. 8d, Patterned top-bottom droplet pair with different sizes. From left to right: schematic of the pattern from XY dimensions with a bottom view, fluorescence image of the bottom view, schematic of the pattern from XZ dimensions, reconstructed 3D fluorescent image, and profile diagram of both green and red fluorescence intensity along the dashed line (second image). FIG. 8e, Patterned three-droplet assemblies. Top row: patterned left-middle-right droplet assembly with different sizes. Bottom row: patterned bottom-middle-top droplet assembly with different sizes. For both patterns, from left to right: schematic of the pattern from XY dimensions with a bottom view, fluorescence image of the bottom view, schematic of the pattern from XZ dimensions, and reconstructed 3D fluorescent image. Scale bars are all 100 μm.

Printed Functional Astrocytes in Droplet Arrays

The printed astrocytes have showed polarised cell morphology, compared to unhealthy cell morphology when astrocytes were printed with agarose as ECM. Printed astrocytes also had higher proportions of cells exhibited spontaneous calcium fluctuations compared to 2D culture, indicating that cells in printed droplets have physiological relevant 3D microenvironment.

Astrocytes and metastatic cancer cells (MDA) were patterned either left-right or top-bottom. Astrocytes showed decreased frequency of spontaneous calcium fluctuations in both patterns, due to the interaction with cancer cells. Further, designed patterns with different amount of cell-cell interaction between the two cell types led to different intracellular calcium levels in astrocytes, indicating a functional response of astrocytes in contact with MDA cells.

We demonstrated that astrocytes shows different functional activity in different cell microenvironment. Astrocytes printed in low 3D droplets alone or together with NPCs exhibited increased spontaneous calcium fluctuations compared to 2D culture. On the contrast, astrocytes with MDA, metastatic breast cancer cells, showed decreased frequency of spontaneous calcium fluctuations. This result is consistent with literature observations that metastatic MDA are be able to interact with astrocytes through both direct cell-cell contact and secreted cytokines.^{4,5} These results indicate the potential cell-cell and cell-ECM interactions influenced by different cell microenvironment generated in these printed and miniature neural tissues.

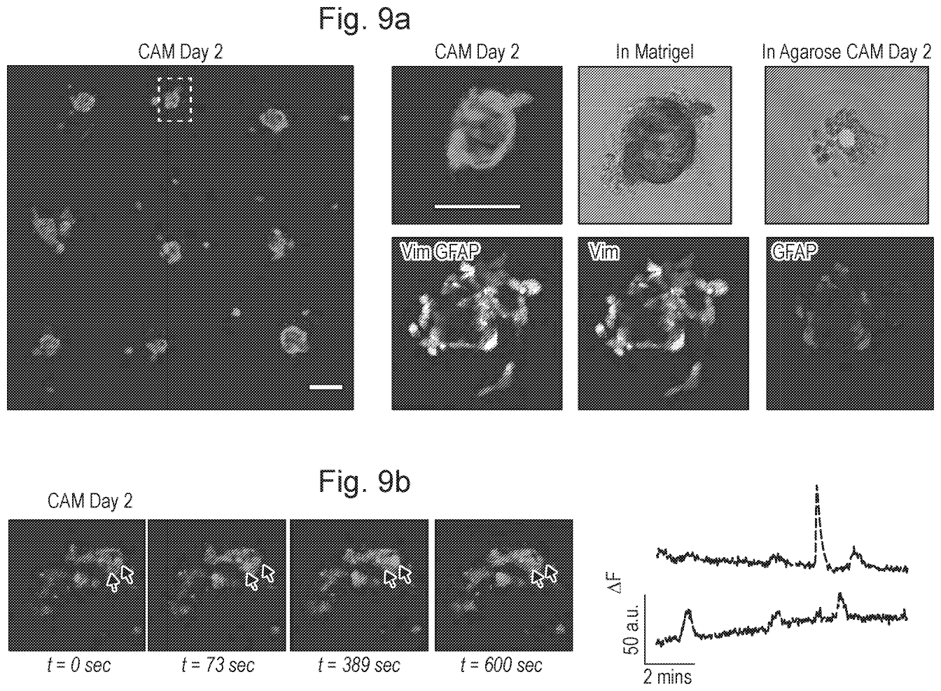

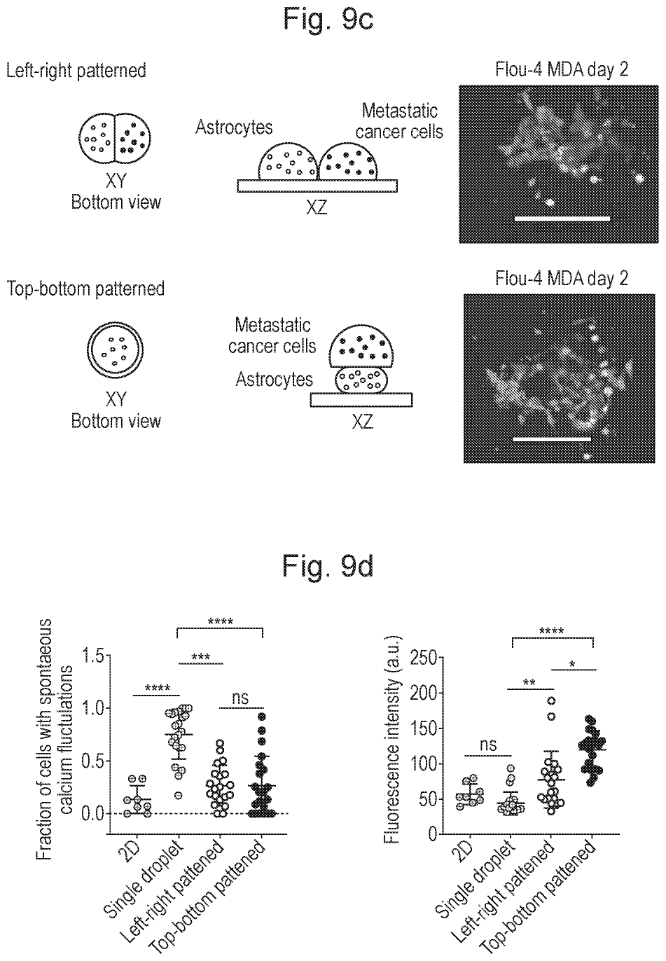

FIG. 9 shows the results for printed cortical astrocyte arrays for functional assays. FIG. 9a, Printed human primary cortical astrocyte array. Astrocytes were printed at a cell density of 4×10⁷/mL in Matrigel. Left, Calcein AM (CAM) staining of printed astrocytes at 2 days post printing. The dashed box indicates the area of the higher magnification image on the right (top two images in the middle). Top right row: higher magnification images of astrocyte printed in Matrigel (CAM staining image and the corresponding brightfield image) and image of astrocytes printed in agarose as the matrix. Bottom right row: immunostaining images of printed astrocytes with astrocyte markers vimentin (yellow) and GFAP (magenta). FIG. 9b, Spontaneous calcium fluctuations of astrocytes in printed droplet arrays. Left: Individual time-point frames of Fluo-4 calcium live imaging. Right: single-cell traces of the cells indicated by the colour-coded arrows in the images (left). FIG. 9c, Patterned droplet pairs with different droplet interface areas. Droplets contained either astrocytes (CAM stained, green) or metastatic cancer cells, MDA (RFP labelled, Red). Top row: left-right patterned. bottom row: top-bottom patterned. For both patterns from left to right: schematic of the patterned droplet pair from XY dimensions with a bottom view, schematic of the pattern from XZ dimensions, and the fluorescent image at 2 days post printing. Right, schematic of top-bottom patterned droplet pair and the fluorescent image (bottom view) at 2 days post printing. Notably, the top-bottom patterned droplet pair has a lager droplet contacting area compared to the left-right patterned droplet air, presumably due to wetting effect with the printing substrate and gravity. FIG. 9d, Scatter plots of the proportions of astrocytes with spontaneous calcium fluctuations (left) and the intracellular calcium concentrations (indicated by the fluorescence intensity of Fluo-4) of astrocytes at different conditions: 2D monolayer cell culture, printed single astrocyte droplet arrays, printed left-right patterned (astrocyte left, MDA right) droplet arrays and printed top-bottom patterned (astrocyte bottom, MDA top) droplet arrays. n>20 for all conditions. *: P<0.05, **: P<0.01, ***: P<0.001 and ****: P<0.0001. Scale bars are all 100 μm in FIG. 9a and 200 μm in FIG. 9c.

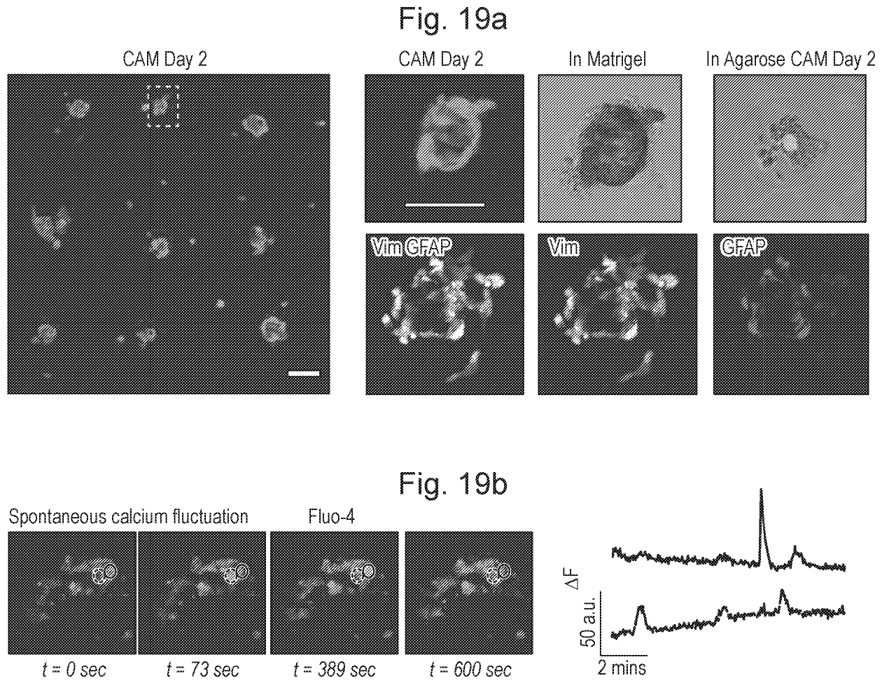

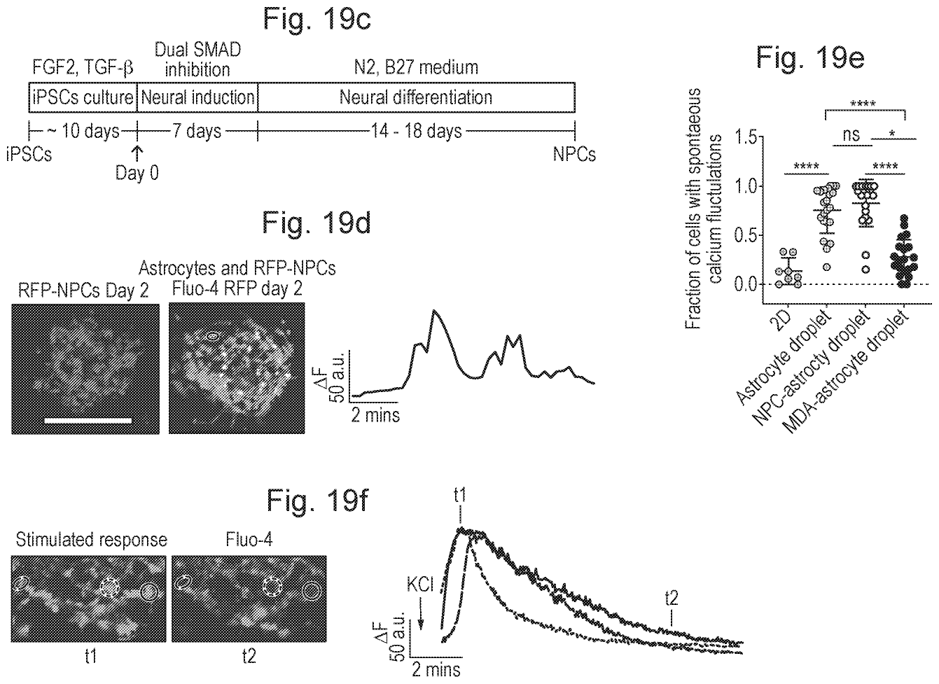

FIG. 19 shows functional and miniature neural tissues from printed arrays. FIG. 19a, Human primary cortical astrocyte printed in an array. Astrocytes were printed at a cell density of 4×10⁷ mL⁻¹ in Matrigel. Left: Calcein AM (CAM) staining of printed astrocytes at 2 days post printing. The dashed box indicates the area of the higher magnification image on the right (top two images in the middle). Top right row: higher magnification images of astrocyte printed in Matrigel (CAM staining image and the corresponding brightfield image) and image of astrocytes (brightfield with CAM staining) printed in agarose as the matrix. Bottom right row: immunostaining images of printed astrocytes with astrocyte markers vimentin (yellow) and GFAP (magenta). FIG. 19b, Spontaneous calcium fluctuations of astrocytes in printed droplet arrays. Left: Individual time-point frames of Fluo-4 calcium live imaging. Right: single-cell traces of the cells indicated by the colour-coded circles in the images (left panel). FIG. 19c, Neural differentiation process from iPSCs to NPCs. iPSCs were cultured with growth factors, FGF and TGF-β, to maintain the pluripotency of the stem cells. Neural induction were induced with two SMAD inhibitors, SB431542 and LDN193189, over 7 days. Further neural differentiation were conducted with neural maintenance medium containing N2 and B27. FIG. 19d, Spontaneous calcium fluctuation of astrocytes when they were co-cultured with RFP-NPCs. Left: an example droplet containing RFP-NPCs at day 2 post printing. Middle: an example droplet containing astrocytes and RFP-NPCs (3:1 cell density ratio and 4×10⁷ mL⁻¹ total cell density). Right: single-cell trace of the cell indicated by the circle in the image. FIG. 19e, Scatter plots demonstrated the differences in cell function of astrocytes under different cell microenvironment. Proportions of astrocytes with spontaneous calcium fluctuations were measured at four conditions: 2D monolayer cell culture, and printed astrocyte, astrocyte-NPC, and astrocyte-MDA (a metastatic breast cancer cell line) droplet arrays. n>20 for all four conditions. *: P<0.05, **: P<0.01, ***: P<0.001 and ****: P<0.0001. FIG. 19f, Astrocytes in droplet arrays were responsive to KCl (60 mM) stimulation. Left two images: two time-point (t1 and t2) frames of Fluo-4 calcium live imaging of neural cells response to KCl stimulation. Right: single-cell traces of the cells indicated by the colour-coded circles in the images (left panel) after KCl stimulation. Scale bars are all 100 μm in FIG. 19a and 150 μm in FIG. 19d.

Neurotoxicity Test Using Printed Neural Tissue Arrays

Neurotoxicity of cancer chemotherapy drugs occurs frequently in clinic and often limit the dose of chemotherapy. We tested three chemotherapy drugs that are reported in clinic with low, moderate and high risks of neurotoxicity, using printed droplet arrays containing neural tissues (astrocytes and NSCs in Matrigel). Our result showed similar toxicity result of three drugs, 5-Fluorouracil, Carboplatin and Taxol, as clinic observations. Importantly, we found that neurons are more sensitive to the drugs compared to astrocytes. Therefore, our technique which uses pico- to nano-liter tissues for bioassays offers the potential for low-cost HTS using expensive human stem cells.

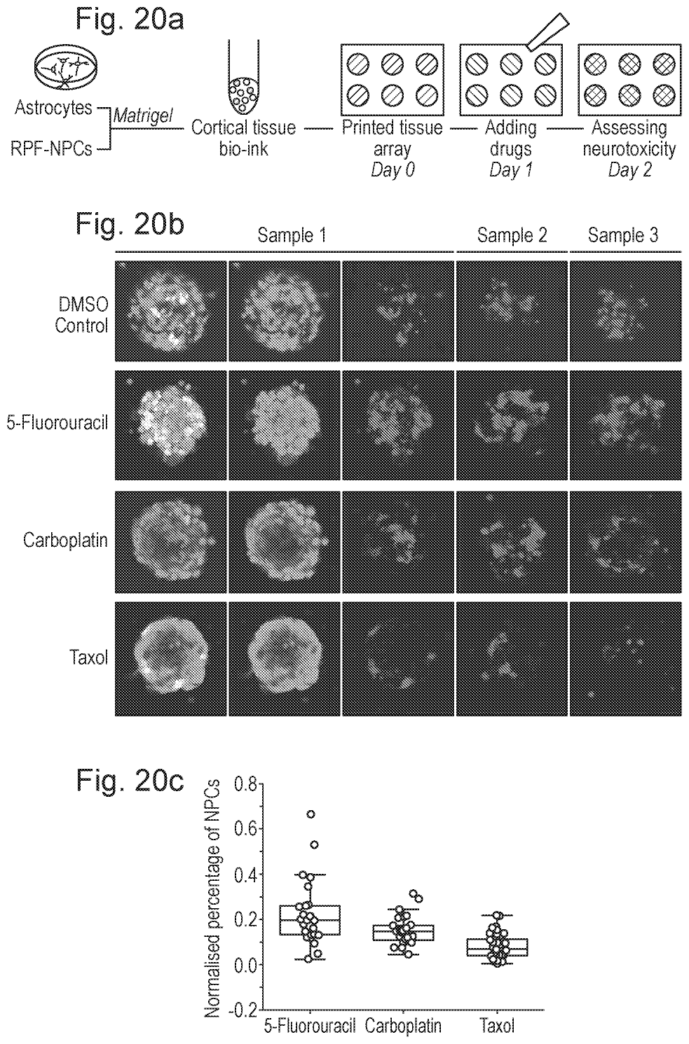

FIG. 20 shows the results for neurotoxicity testing using printed neural tissue arrays. FIG. 20a, The experimental process of drug testing using printed neural tissue arrays. From left to right: 2D cultured astrocytes and RFP-NPCs were harvested and added to Matrigel for the preparation of bio-ink with a cell density ratio of 3:1 (astrocytes vs RFP-NPCs) and total cell density of 4×10⁷ mL⁻¹. The prepared bio-ink were printed into droplet arrays with individual droplet size around 100 μm. On the next day, cancer chemotherapy drugs were added to the culture medium. The neurotoxicity of the drugs were assessed after a further 24 hrs. FIG. 20b, Fluorescent images of neural tissues treated with DMSO (control) and cancer chemotherapy drugs with different reported neurotoxicity risks: 5-Fluorouracil (minor), Carboplatin (moderate), Taxol (high). Neurotoxicity against neural progenitor cells (RFP-NPCs in red) was observed. FIG. 20c, Comparison of the neurotoxicity of different chemotherapy drugs. Consistent with clinic observations, Taxol and Carboplatin showed higher toxicity against neural cells compared to 5-Fluorouracil. n>25, P<0.05.

Neural Migration in Printed Nano-Bioreactors

Bioreactors have been used in macro-scale to support biological active environment. However, nanoliter-sized bioreactors would offer the potential to study biological events with much small number of cells, and the potential application in high throughput assays. We firstly optimised the oil surrounding the nano-bioreactors, which plays a large role in the viability of the encapsulated neural tissues. In this regard, for neural progenitor cells it was found that sunflower oil was non-toxic to NPCs. It was also found that the key component of sunflower oil, glyceryl trioleate was not toxic to NPCs. The optimised conditions allow the growth of individual low-nanoliter sized neural tissue inside each nano-bioreactor, without interaction with cells in other nano-bioreactors. With differentiation medium, neural process outgrowth and neural migration were observed and assessed in nano-bioreactors. This technique have the potential for a low-cost HTS, as minimal cells and reagents are required. It also offers the potential application to study biological events where a small organised cluster of cells were involved, such as early stage embryogenesis and tumorigenesis.

FIG. 21 shows neural migration in printed nano-bioreactors. FIG. 21a, A sequential printing process for the production of nano-bioreactors. Left to right: pL to nL droplets, containing cells and ECM, were printed in glyceryl trioleate, an optimised oil condition; after gelation at 37° C. for 10 mins, defined culture medium was printed to encapsulate the tissue; biological events were then assessed in the nano-bioreactors. FIG. 21b, Schematic (left) and image (right) show the printed nano-bioreactors with defined sizes. FIG. 21c, Schematic of neural migration observed in nano-bioreactors with patterned tissues where RFP-NPCs were encapsulated in ECM. Firstly, the neural cells (RFP-NPCs) projected processes into the ECM compartment, followed by cell migration. FIG. 21d, Neural migration at day 1, 4, and 6 in nano-bioreactor. Dashed boxes indicate the regions with the higher magnification images in the bottom row. Dashed lines in the bottom row images show the boundary of the NPC and ECM compartments. The white arrows indicating the migrating cells along neural processes. FIG. 21e, Quantification of neural migration in the printed nano-bioreactors. Increasing migration distance was observed over the first 4 days post printing. n=13, P<0.05. Scale bars are 200 μm and 100 μm in the top and bottom rows of FIG. 21d respectively.

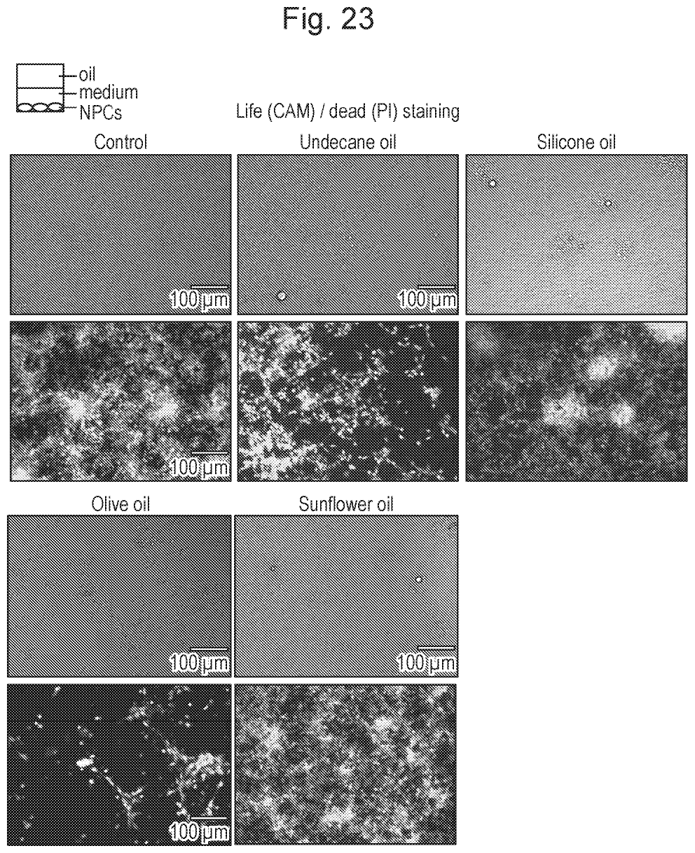

FIG. 23 shows examples from the oil toxicity test using iPSCs derived NPCs. 2D cultured NPCs were added with oil on top of medium and cultured for 5 days. Life (CAM, in green) and Dead (PI, in read) staining were conducted on day 5. No obvious toxicity was observed with sunflower oil.

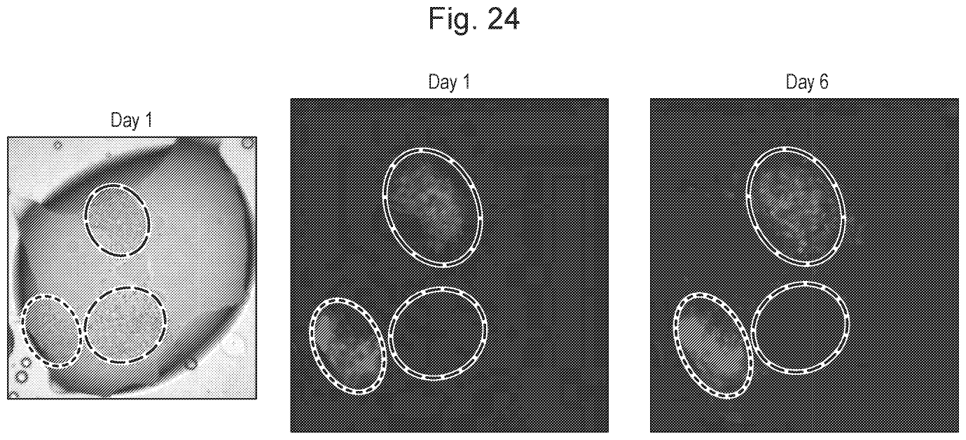

FIG. 24 shows the results from printing multiple droplets containing different cells in nano-bioreactors. Printed three droplets in one nano-bioreactor: two droplets containing RFP—NPCs and the third droplets containing astrocytes (no colour). This could be used for the investigation of secretory effects between different cell types at nanoliter scale.

### REFERENCES (EXAMPLE 2)

1. Jang, M.; Koh, I.; Lee, S. J.; Cheong, J. H.; Kim, P., Droplet-based microtumor model to assess cell-ECM interactions and drug resistance of gastric cancer cells. Sci Rep 2017, 7, 41541.
2. Cole, R. H.; Tang, S. Y.; Siltaneni, C. A.; Shahi, P.; Zhang, J. Q.; Poust, S.; Gartner, Z. J.; Abate, A. R., Printed droplet microfluidics for on demand dispensing of picoliter droplets and cells. Proc Natl Acad Sci USA 2017, 114 (33), 8728-8733.
3. Chen, Q.; Utech, S.; Chen, D.; Prodanovic, R.; Lin, J. M.; Weitz, D. A., Controlled assembly of heterotypic cells in a core-shell scaffold: organ in a droplet. Lab on a chip 2016, 16 (8), 1346-9.
4. Gong, X. H.; Hou, Z. M.; Endsley, M. P.; Gronseth, E. I.; Rarick, K. R.; Jorns, J. M.; Yang, Q. H.; Du, Z. G.; Yan, K.; Bordas, M. L.; Gershan, J.; Deepak, P.; Geethadevi, A.; Chaluvally-Raghavan, P.; Fan, Y. B.; Harder, D. R.; Ramchandrar, R.; Wang, L., Interaction of tumor cells and astrocytes promotes breast cancer brain metastases through TGF-beta 2/ANGPTL4 axes. Npj Precis Oncol 2019, 3.
5. Kim, S. J.; Kim, J. S.; Park, E. S.; Lee, J. S.; Lin, Q. T.; Langley, R. R.; Maya, M.; He, J. Q.; Kim, S. W.; Weihua, Z.; Balasubramanian, K.; Fan, D.; Mills, G. B.; Hung, M. C.; Fidler, I. J., Astrocytes Upregulate Survival Genes in Tumor Cells and Induce Protection from Chemotherapy. Neoplasia 2011, 13 (3), 286-298.

1. An apparatus for producing a droplet assembly,
which apparatus comprises:
at least one droplet generator suitable for generating droplets of a viscous droplet medium;
a droplet receiving region which is moveable relative to the at least one droplet generator;
a temperature controller; and
a control unit, which control unit is adapted to control the dispensing of droplets from the at least one droplet generator and the movement of the droplet receiving region relative to the at least one droplet generator,
wherein the apparatus is adapted to produce a droplet assembly in the droplet receiving region,
wherein the droplet assembly comprises a plurality of droplets, wherein each of said droplets comprises a droplet medium.

2. An apparatus according to claim 1 wherein the at least one droplet generator is suitable for generating droplets of a droplet medium which has viscosity, measured at the temperature of the droplet medium in the droplet generator at the time of generating a droplet thereof, of at least 50 mPa s, preferably at least 75 mPa·s, more preferably 100 mPa·s, optionally from 100 mPa·s to 10 Pa·s,
optionally wherein the droplet generator contains said droplet medium, preferably wherein the droplet medium has a viscosity, measured at a temperature of 5° C., of at least 50 mPa·s, preferably at least 75 mPa·s, preferably at least 100 mPa·s, optionally from 100 mPa·s to 10 Pa·s.

3. An apparatus according to claim 1 or claim 2 wherein the droplet medium comprises a natural extracellular matrix material, optionally wherein the natural extracellular matrix material is matrigel,
preferably wherein at least 80% by volume of the droplet medium is said natural extracellular matrix material,
more preferably wherein at least 90% by volume of the droplet medium is said natural extracellular matrix material.

4. An apparatus according to any one of the preceding claims wherein the droplet medium comprises an undiluted natural extracellular matrix material and biological cells,
more preferably wherein the droplet medium consists of a natural extracellular matrix material and biological cells, preferably wherein the natural extracellular matrix material is matrigel.

5. An apparatus according to any one of the preceding claims wherein the or each droplet generator comprises: a chamber for holding droplet medium; an outlet; and a component for displacing a volume of said droplet medium through said outlet and thereby dispensing said volume as a droplet,
optionally wherein the component for displacing a volume of said droplet medium through said outlet is a piezoelectric transducer,
preferably wherein the apparatus further comprises a piezoelectric driver which is capable of applying voltages more negative than −50 V and voltages more positive than +50 V to the piezoelectric transducer, preferably wherein the piezoelectric driver is capable of applying voltages more negative than −100 V and voltages more positive than +100 V to the piezoelectric transducer, more preferably wherein the piezoelectric driver is capable of applying voltages more negative than −120 V and voltages more positive than +120 V to the piezoelectric transducer, optionally wherein the piezoelectric driver is capable of applying voltages of −130 V and +130 V to the piezoelectric transducer.

6. An apparatus according to any preceding claim wherein the droplet receiving region further comprises a bulk medium, wherein the bulk medium and the droplet medium are immiscible and wherein the temperature controller is for maintaining the temperature of the bulk medium,
optionally wherein the temperature controller is capable of maintaining the temperature of the bulk medium at least at any temperature in the range of from −5° C. to +40° C., optionally wherein the temperature controller is capable of maintaining the temperature of the bulk medium at least at any temperature in the range of from −15° C. to +80° C.

7. An apparatus according to claim 5 or claim 6 wherein a surface of the outlet, preferably an inside surface of the outlet, has a hydrophilic surface treatment,
preferably wherein the hydrophilic surface treatment provides said surface of the outlet with a positive charge,
more preferably wherein the hydrophilic surface treatment comprises (3-aminopropyl)trimethyoxysilane.

8. A process for producing a droplet assembly using an apparatus for producing the droplet assembly, which droplet assembly comprises: a plurality of droplets, wherein each of said droplets comprises a droplet medium;
which apparatus comprises:
at least one droplet generator wherein the droplet generator is suitable for generating droplets of a viscous droplet medium;
a droplet receiving region which is moveable relative to the at least one droplet generator;
a temperature controller; and
a control unit, which control unit is adapted to control the dispensing of droplets from the at least one droplet generator and the movement of the droplet receiving region relative to the at least one droplet generator;
wherein said droplet receiving region further comprises a bulk medium, wherein the bulk medium and the droplet medium are immiscible; which process comprises:
(a) a plurality of dispensing steps, wherein each dispensing step comprises dispensing a droplet of the droplet medium from a said droplet generator into the bulk medium, and thereby forming in the bulk medium a droplet which comprises said droplet medium; and
(b) moving the droplet receiving region relative to the at least one droplet generator, to control the relative positioning of the droplets in the bulk medium.

9. A process according to claim 8 which droplet assembly comprises: a plurality of droplets, wherein each of said droplets comprises: (i) a droplet medium, and (ii) an outer layer of amphipathic molecules around the surface of the droplet medium;
which process comprises: (a) a plurality of dispensing steps, wherein each dispensing step comprises dispensing a droplet of the droplet medium from a said droplet generator into the bulk medium, in the presence of amphipathic molecules, and thereby forming in the bulk medium a droplet which comprises (i) said droplet medium and (ii) an outer layer of amphipathic molecules around the surface of the droplet medium; and (b) moving the droplet receiving region relative to the at least one droplet generator, to control the relative positioning of the droplets in the bulk medium;
and wherein:
the at least one droplet generator is a piezoelectric droplet generator which comprises a piezoelectric transducer for dispensing droplets, and wherein each dispensing step comprises applying a voltage pulse to the piezoelectric transducer, wherein applying the voltage pulse comprises applying a voltage more negative than −100 V or a voltage more positive than +100 V to the piezoelectric transducer, and the voltage pulse has a peak-to-peak amplitude of at least 200 V; and
during each dispensing step, the temperature controller maintains the temperature of the bulk medium at a droplet printing temperature; and
preferably wherein the apparatus is as defined in any one of claims 1 to 7.

10. A droplet assembly which is obtainable by a process as defined in any one of claims 8 or 9.

11. A process for producing a droplet assembly, the process comprising generating, in a bulk medium, a plurality of droplets, wherein each of said droplets comprises: (i) a droplet medium which comprises biological cells and a natural extracellular matrix material, and (ii) an outer layer of amphipathic molecules around the surface of the droplet medium, wherein the bulk medium and the droplet medium are immiscible, and contacting each of said droplets with another of said droplets to form a layer of said amphipathic molecules as an interface between contacting droplets.

12. A droplet assembly which is obtainable by a process as defined in claim 11.

13. A process for producing a pre-patterned tissue construct, the process comprising
producing a pre-patterned droplet assembly in a bulk medium by a process as defined in any one of claims 8, 9 and 11, or providing a pre-patterned droplet assembly as defined in any one of claims 10, 12 and 17, provided that, in the pre-patterned droplet assembly, the droplet medium comprises natural extracellular matrix material and biological cells;
gelling the natural extracellular matrix material to produce a pre-patterned tissue construct which comprises gelled natural extracellular matrix material and the biological cells; and
recovering the pre-patterned tissue construct from the bulk medium.

14. A pre-patterned tissue construct which is obtainable by a process as defined in claim 13.

15. A process for producing a cultured tissue construct comprising
preparing a pre-patterned tissue construct by the process of claim 13; and
culturing the biological cells in the pre-patterned tissue construct.

16. A cultured tissue construct which is obtainable by the process of claim 15.

17. A droplet assembly comprising: a plurality of droplets in contact with one another, wherein each of said droplets comprises: (i) a droplet medium which comprises biological cells and a natural extracellular matrix material, and (ii) an outer layer of amphipathic molecules around the surface of the droplet medium, wherein each of said droplets contacts another of said droplets to form a layer of said amphipathic molecules as an interface between the contacting droplets.

18. A pre-patterned tissue construct comprising a plurality of gelled droplets in contact with one another, wherein each of said droplets comprises a droplet medium which comprises biological cells and a gelled natural extracellular matrix material, and wherein each of said droplets is adhered to another of said droplets by the gelled natural extracellular matrix material.

19. A cultured tissue construct which comprises a natural extracellular matrix material and biological cells, wherein the cultured tissue construct is obtainable by providing a pre-patterned tissue construct as defined in claim 14 or 18 and culturing the biological cells in the pre-patterned tissue construct.

20. A droplet array which comprises a plurality of elements spaced apart from one another on a substrate in a bulk medium, wherein each element comprises at least one droplet which comprises a droplet medium which comprises one or more biological cells and a natural extracellular matrix material, wherein the bulk medium and the droplet medium are immiscible,
optionally wherein each element comprises at least one droplet which comprises (i) a droplet medium which comprises one or more biological cells and a natural extracellular matrix material and (ii) an outer layer of amphipathic molecules.

21. A droplet array which comprises a plurality of elements spaced apart from one another on a substrate, wherein each element comprises at least one gelled droplet, wherein each gelled droplet comprises a droplet medium which comprises one or more biological cells and a gelled natural extracellular matrix material.

22. Use of a droplet array as defined in any one of claims 20, 21 and 25 in high throughput screening.

23. A method of screening a test substance which comprises providing an array as defined in any one of claims 20, 21 and 25, contacting the test substance with at least one of the elements of the array, and measuring a response.

24. A process for producing a droplet array, which droplet array comprises a plurality of elements spaced apart from one another on a substrate in a bulk medium, wherein each element comprises at least one droplet which comprises a droplet medium which comprises one or more biological cells and a natural extracellular matrix material, wherein the bulk medium and the droplet medium are immiscible; which process comprises generating a plurality of droplets in the bulk medium, wherein each of said droplets comprises a droplet medium which comprises one or more biological cells and a natural extracellular matrix material, and arranging the droplets on the substrate in the bulk medium to form said plurality of elements spaced apart from one another, wherein each element comprises at least one of said droplets.

25. A droplet array which is obtainable by the process of claim 24.

26. A nano-bioreactor comprising at least one droplet which comprises a droplet medium which comprises one or more biological cells and a natural extracellular matrix material, and at least one droplet of culture medium.

27. An array of nano-bioreactors comprising a plurality of nano-bioreactors spaced apart from one another on a substrate in a bulk medium, wherein each nano-bioreactor comprises at least one droplet which comprises a droplet medium which comprises one or more biological cells and a natural extracellular matrix material, and at least one droplet of culture medium, wherein the bulk medium and the droplet medium are immiscible,
preferably wherein the bulk medium and the culture medium are immiscible.
